(19) United States
(12) Patent Application Publication
(10) Pub. No.: US 2004/0040047 A1
(43) Pub. Date: Feb. 26, 2004

(54) Regulated apoptosis using chemically induced dimerization of apoptosis factors

(76) Inventors: David M. Spencer, Houston, TX (US); Kevin M. Slawin, Houston, TX (US)

Correspondence Address: FULBRIGHT & JAWORSKI, LLP, 1301 MCKINNEY, SUITE 5100, HOUSTON, TX, 77010-3095, US

(21) Appl. No.: 10/247,019

(22) Filed: Sep. 19, 2002

Related U.S. Application Data
(63) Continuation of application No. 09/647,418, filed on Sep. 29, 2000, now abandoned.
Application No. 09/647,418 is a 371 of international application No. PCT/US99/06799, filed on Mar. 30, 1999.
(60) Provisional application No. 60/079,831, filed on Mar. 30, 1998.

(51) Int. Cl. A01K 67/00; A61K 48/00; C12N 15/00

(52) U.S. Cl. 800/8; 514/44; 435/320.1

(57) ABSTRACT

The present invention discloses artificial death switches (ADSs) based on chemically induced dimerization of the cysteine proteases, caspase-1 (ICE) and caspase-3 (YAMA). In both cases, aggregation of the target protein is achieved by a non-toxic, lipid-permeable, dimeric FK506 analog that binds to an attached FK506-binding protein (FKBP). The intracellular crosslinking of caspase-1 or caspase-3 is sufficient to trigger rapid apoptosis in a Bcl-xL-independent manner, suggesting that these conditional pro-apoptotic molecules can bypass intracellular checkpoint genes, like Bcl-xL, that limit apoptosis. Since these chimeric molecules are derived from autologous proteins, they should be non-immunogenic and thus ideal for long-lived gene therapy vectors. These properties should also make chemically-induced apoptosis (CIA) useful for developmental studies, for treating hyperproliferative disorders and for developing animal models to a wide variety of diseases.

### BACKGROUND OF THE INVENTION

1. Field of the Invention

The present invention is directed to the field of molecular biology and, in particular, the fields of regulated apoptosis and gene therapy.

2. Description of the Related Technology

Over the past several years gene therapy has been evolving as a therapeutic option for numerous benign and malignant human diseases. Approaches to gene therapy fall into several broadly defined categories including: gene replacement therapy for diseases caused by the absence or malfunction of a single gene; immune system activation and vaccine development; and conditionally lethal gene therapy also known as “suicide gene” therapy. An example of a commonly used conditionally lethal gene frequently used for gene therapy of malignant diseases is the thymidine kinase (tk) gene from Herpes simplex virus (HSV). As used in a gene therapy application, the tk gene may be incorporated into a gene therapy and upon introduction of the gene therapy vector into a cell, a copy of the tk gene is introduced into the target cell. The presence of the tk gene renders the cells sensitive to the dideoxy nucleoside analog ganciclovir. When cells expressing tk are contacted with ganciclovir, the tk gene phosphorylates the nucleoside analog resulting in a form of the compound that can be further processed and incorporated into elongating DNA, leading to chain termination (3). Cells lacking the tk gene do not process ganciclovir and thus are not affected. Other genes encoding different enzymatic activities have been used as suicide genes. These include the E. coli purine nucleoside phosphorylase E gene, which generates toxic purines, and the bacterial cytosine deaminase gene which converts 5-fluorocytosine to 5-fluorouracil. Both of these genes function by the in situ conversion of a nucleoside analogue into a form that is incorporated into replicating DNA thereby interfering with the replication process. Other conditionally lethal genes that have been employed in gene therapy applications include the E. coli nitroreductase gene (see Drabek, et al. Gene Therapy 4(2):93-100, 1997) that acts by converting the pro-drug CB 1954 into a cytotoxic DNA interstrand crosslinking agent and the hepatic cytochrome P450 2B1 (see Wei, et al. Human Gene Therapy 5(8):969-978, 1994) that acts by converting the anticancer drug cyclophospharmide into a toxic DNA-alkylating agent.

A problem inherent in all of these systems is the toxic and/or mutagenic nature of the pro-drugs employed. In all of the systems just mentioned, the pro-drug, even prior to its conversion into the active form, can have deleterious effects on cells. As a result of this toxicity, these types of gene therapy systems are not appropriate unless the condition of the patient warrants assuming the risks of therapy. Thus, these systems are completely inappropriate for treatment of benign hyperproliferative conditions. Notwithstanding these limitations, vectors incorporating these genes have been developed and tested on various tumor models.

An example of such use of the thymidine kinase gene is provided in U.S. Pat. No. 5,631,236, issued to Woo, et al. which is specifically incorporated herein by reference. The Woo, et al. patent discloses a method for treating localized solid tumors and papillomas in an individual. The method disclosed by Woo, et al. comprises introducing a recombinant adenoviral vector containing the Herpes simplex virus thymidine kinase gene. Subsequently, the infected cells are treated with the drug ganciclovir resulting in the death of the cells expressing thymidine kinase. Woo, et al. disclosed the use of their adenoviral construct for the treatment of various types of cancers and papillomas including colon carcinoma, prostate cancer, breast cancer, lung cancer, melanoma, hepatoma, brain, head and neck cancers.

One result that has emerged from these tests is the observation that it is not necessary to introduce a suicide gene into every tumor cell in order to effect reduction in tumor size. In fact, the successful introduction of the suicide gene into a small fraction of the tumor cells may result in an overall regression of the tumor. This effect, the death of cells not expressing the suicide gene in response to the death of a small percentage of cells carrying this suicide gene, has been termed the “bystander effect.” The bystander effect has been observed in a wide variety of tumor model system such as rat glioma, mouse model of prostate cancer, a mouse model of squamous cell carcinoma and others.

The bystander effect has been observed, not only within the tumor into which the suicide gene has been transduced, but in tumors located distally to the transduced tumor. In a mouse model for squamous cell carcinoma, Wilson, et al. (Arch. Otolar. Head and Neck Surgery 122:746-749, 1996) induced tumors in mice by injection of UMSCC 29 cells (No. 29 cells) into both the left and right flanks of mice. The tumors were allowed to grow and the tumors in the left flanks were subsequently injected with cells producing a retrovirus expressing HSV-tk while the tumors in the right flanks were not treated. After two days were allowed for in vivo transduction of the HSV-tk gene into the tumor cell genome, treatment with ganciclovir was begun. The authors observed a regression in the tumors located in the untreated flank after the commencement of ganciclovir treatments. Thus, killing of tumors at one site can result in the killing of tumor cells at a distant location; even though the distant cells were not capable of converting ganciclovir into a lethal form.

Several explanations have been proposed for the bystander effect. First, the cells treated with the suicide gene may release some factor that is toxic to adjacent tumor cells. The factor might be produced in response to the infection or might be a metabolite of ganciclovir. This explanation does not appear to account for the distal effects recounted above. A second hypothesis is that, as a result of the apoptotic or necrotic process that commences upon the administration of ganciclovir, certain toxic substances might be released by the affected cells which results in the death of the adjacent cells. Once again it is difficult to explain distal effects in the context of this model. A third hypothesis is that the death of the infected cells somehow potentiates an immune response to the tumor cells, perhaps by inducing an immune response to a tumor-specific antigen. This model could explain the distal effects observed. One mechanism that is envisioned is the uptake of apoptotic or necrotic tumor cell components by antigen-presenting cells thereby inducing a cytotoxic T cell response to cells expressing the antigen (Albert Nature 392:86-89, 1998).

There is evidence that some immune component is required for the bystander effect. In a mouse model employing carcinoma cell line MC26, Gagandeep, et al. (Cancer Gene Therapy 3(2): 83-88, 1996) observed almost total tumor regression in immunocompetent BALB/c mice, but not in immunocompromised athymic BALB/c mice, when MC26 cells were co-injected with a retroviral packaging line expressing a HSV-tk gene and subsequently treated with ganciclovir. The immunological component may also provide protection against subsequent challenge by cancer cells as shown by Hall, et al. (International Journal of Cancer 70:183-187, 1997). Subcutaneous tumors were induced in mice by the injection of RM-1 cells, a tumor cell line of prostate origin. The tumors were subsequently treated with a replication deficient adenovirus expressing HSV-tk under the control of the RSV promoter, followed by treatment with ganciclovir. After treatment with ganciclovir, tumors were surgically removed and the mice were challenged by RM-1 cells injected into the tail vein. The animals were subsequently sacrificed and the lungs analyzed for visible lung metastases. A 40% reduction in lung colonization in the treatment group indicated the possible production of a systemic, anti-metastatic activity following a single treatment with an adenovirus expressing HSV-tk and ganciclovir. These results lead to the conclusion that it may be possible to treat not only solid, single tumors with suicide gene therapy but also metastatic conditions.

Despite the successes observed in these and other systems, several problems remain with the suicide genes known in the art. A major problem is the xenogeneic nature of the suicide gene itself Suicide genes of the prior art are of bacterial or viral origin. This typically results in the mounting of an immune response against cells expressing the suicide gene, reducing the prospects for long-term expression and shifting the immune response from putative tumor-specific antigens towards potentially immunodominant peptide epitopes of the suicide genes themselves.

Another major limitation in the suicide genes of the prior art is the nature of their cytotoxic activity. The suicide genes of the prior art are directed to interfering with DNA replication/maintenance. For example, the herpes virus tk gene results in the production within the cell of a nucleoside analogue which is incorporated into DNA and results in chain termination. This means that the efficacy of the therapy is dependent entirely upon the incorporation of the nucleoside analogue into the DNA of the target cell. While therapies based on this have shown to be effective in cells that are rapidly replicating, i.e., cancer cells, these types of methods are far less effective on non-replicating cells.

An additional complexity that has been observed in therapeutic modalities based on the suicide genes of the prior art is the variable nature of the effects of the suicide gene on different cell types. Beck, et al. (Human Gene Therapy 6:1525-1530, 1995) tested the sensitivity of a variety of tumor cell types to the effects of herpes virus tk/ganciclovir mediated cell death. The amount of ganciclovir required and the length of exposure required to induce cell death was highly variable in vitro, and this variability was found to correlate with the in vivo effectiveness of the treatment. The time necessary to effect a substantial amount of cell death varied from 6 to 22 days at high doses of ganciclovir. Even more variable results were obtained at a lower dose of ganciclovir with one cell line (ESB, FIG. 1) which was essentially immune to doses of ganciclovir up to 1 μg/mL. An additional difficulty was revealed by Beck's work. It took up to three weeks to obtain a high level of cell killing even at high doses of ganciclovir. This exposure to high doses of potentially toxic materials for protracted periods of time may have significant side effects.

Despite the problems seen with the suicide genes of the prior art, they have found widespread acceptance and have been incorporated into a wide variety of therapeutic modalities. Gene therapy applications based on the herpes virus tk gene have been developed for prostate cancer, gliomas, head and neck squamous cell carcinomas, breast cancer, and a host of solid tumors. These therapies have been tested in a variety of model systems and some have progressed to the point of evaluation in human patients. A protocol based on the delivery of herpes virus tk gene to prostate cancer using an adenovirus vector has been developed at Baylor College of Medicine and has completed phase I trials. Of the 18 patients tested, three demonstrated a greater than 50% drop in their serum prostate specific antigen (PSA) levels and an additional six patients experienced a temporary stabilization in their previously rising PSA levels. Thus, in this limited trial, half of the patients treated experienced some amelioration of disease.

An alternative approach to suicide genes involves using endogenous cellular mechanisms. One example of such a method is the use of Fas-mediated apoptosis. Fas is a member of the tumor necrosis factor receptor (TNFR) superfamily whose members can induce pleiotropic responses, including proliferation, activation, differentiation and apoptosis, depending primarily on their cytoplasmic signaling domains (reviewed in 9). The molecular details of Fas-mediated apoptosis, schematically represented in FIG. 1, are rapidly emerging and frequently reviewed (10).

With reference to FIG. 1, the interaction of Fas with the Fas-ligand (FasL) leads to the aggregation of Fas cytoplasmic death domains (DD) and increases the affinity of the Fas DD for the DD of the adapter molecule, FAS-associating protein with death domain, FADD (MORT1). FADD, in turn, interacts with the cysteine protease, caspase-8 (FLICE/MACH) or related caspase-10, via conserved death effector domains (DEDs) found in both proteins. Thus, Fas crosslinking leads to caspase-8 crosslinking and subsequent activation of the apoptotic cascade.

Like all caspases, caspase-8 is an aspartic acid-directed protease that is activated by the proteolytic removal of its amino terminal pro-domain and by an additional internal cleavage, producing a fully active molecule comprised of two p17 and two p12 subunits. Probably via a mechanism involving transproteolysis, the aggregation of caspase-8 contributes to its activation and the initiation of a protease cascade that includes caspase-1(ICE)related and caspase-3(YAMA/CPP32)-related enzymes, ultimately leading to the irreversible cleavage of multiple pro-apoptotic targets.

The first step of the Fas-mediated apoptotic cascade is the multimerization of Fas. This can be accomplished using anti-Fas antibodies or Fas ligand. Both of these reagents react with the extracellular portion of Fas and result in multimerization, however, since they can react with all cells expressing Fas, they cannot be used to selectively ablate specific genetically altered cells.

The problem of specificity was overcome by constructing a genetically engineered form of Fas that can be selectively dimerized in response to an exogenous ligand by chemically induced dimerization, (CID). CID activation of the Fas-mediated apoptotic cascade is described in WO 95/02684 and in U.S. patent application Ser. No. 08/093,499 and Ser. No. 08/179,143. These three documents are specifically incorporated herein by reference. A schematic representation of this technique is presented in FIG. 2.

With reference to FIG. 2, a recombinant Fas molecule is constructed that lacks an extracellular domain. In addition, the cytoplasmic portion of the Fas molecule is engineered to contain one or more copies of the immunophilin FK506 binding protein 12 (FKBP12). The FKBP12 domain binds with high affinity to the dimerization inducer and thus is referred to as a chemical inducer binding domain or CBD. Further, the recombinant Fas molecule is engineered to contain an N-terminal myristoylation sequence. When contacted with the dimerization inducer FK1012 (a dimeric form of FK506, the structure of which is presented in FIG. 18), the CBD portions of two Fas molecules bind to the same inducer molecule resulting in the aggregation of the Fas molecules. The result of this aggregation is the activation of the Fas-mediated apoptotic cascade as described above.

This methodology suffers from some important drawbacks. A first, major obstacle to the use of Fas-based conditionally lethal constructs is autotoxicity. In the absence of inducer, the Fas constructs of the prior art are toxic to cells expressing them. This limits the amount of Fas construct than can be expressed in any given cell. Limiting the amount of Fas expressed may reduce the efficiency with which apoptosis is induced.

A further difficulty is presented by the endogenous control mechanisms that normally inhibit apoptosis. As the Fas constructs of the prior art initiate the apoptotic cascade at an early point in the Fas-mediated apoptotic pathway, the effects of Fas induction are subject to the intracellular check points that limit apoptosis. Thus, even though addition of the CID results in the dimerization of the intracellular portion of Fas and causes the earliest events in the apoptotic cascade to occur, such as the association of FADD with Fas, the incipient apoptotic cascade may be stopped at a cellular check point before the apoptotic process is completed. Many tumors, especially those resistant to standard chemotherapeutic or hormonal therapies, have been shown to up-regulate the expression of apoptosis inhibiting gene products such as Bcl-2. Thus, the presence of apoptosis inhibiting gene products is likely to limit the use of Fas-based constructs.

Another factor that may limit the utility of Fas-based constructs as gene therapeutics is the requirement for additional, mitochondrial factors and proteins, including cytochrome c, for activation of the most downstream members of the apoptotic protease cascade including caspase-3, caspase-6, and caspase-7 (reviewed in 11). Also, the protease apoptosis inducing factor (AIF) may be essential for the activation of the downstream members. Members of the Bcl-2 family, like Bax or Bcl-x_{L}, are localized primarily in the mitochondria and help to modulate the release of these additional factors. The release of the additional factors is concomitant with an apoptosis-associated increase in the permeability of the mitochondrial membrane (12). The requirement for these additional factors may decrease the efficiency with which Fas-based constructs can induce apoptosis.

Despite these drawbacks, this system has been shown to be able to induce apoptosis in non-proliferating cells including CD4⁺CD8⁺ thymocytes (4,5), differentiated neutrophils and monocytes (6), and hepatocytes (7). This system also has the advantage that conditional Fas alleles can be made from human proteins, minimizing potential immunogenicity (5,8).

The techniques described in the instant application have been developed to overcome the problems and limitations of prior art gene therapy methods. By placing apoptosis inducing factors under the conditional control of chemically inducible dimerization domains, the difficulties experienced in prior art methods have been obviated. The ligand used to induce dimerization is substantially non-toxic, thus eliminating the concerns raised by the use of toxic pro-drugs. The chemically inducible apoptosis constructs of the present invention are nontoxic as well, in contrast to the autotoxicity of Fas-based constructs seen in the prior art. Additionally, and perhaps most importantly, the point of action of the constructs of the present invention is downstream of the apoptosis inhibiting checkpoints, thus, after initiation of the apoptotic cascade by chemically induced dimerization, the constructs of the present invention will not be inhibited by the up-regulation of apoptosis checkpoint genes.

While there is an extensive body of literature describing the use of suicide gene therapy protocols to treat malignant tumors, there are many fewer suicide gene therapy protocols for the treatment of benign proliferative disorders such as benign prostate hyperplasia (BPH). One reason for this is the inherently risky nature of the gene therapy protocols of the prior art due to the toxicity of the molecules used. Since the condition is not life threatening, the use of a treatment modality with such large inherent risks is seldom warranted. In addition, the suicide gene therapy methods of the prior art are most effective against rapidly proliferating cells, such as those found in malignant tumors. In contrast, in most benign hyperproliferative disorders, the rate of cell proliferation is lower than it is in malignant disorders making the suicide gene therapy protocols of the prior art even less appropriate. For example, the enlargement of the prostrate seen in BPH is not a result of increase in the proliferation rate of prostate cells, but rather results from a decrease in the rate of deletion of prostate cells by apoptosis. Thus, the suicide gene therapies of the prior art are not appropriate for use against this condition.

BPH refers to the benign enlargement of the prostate that develops in the aging male. The change that occurs in the prostate with the development of BPH have been described in two distinct stages in the pathologic development of BPH. First, there is the development of nodules in the glandular tissue of the transition zone and in the periurethral region of the prostate which can be seen as early as the fourth decade of life. The number of nodules increases linearly with age, while the size of individual nodules increases slowly. The second stage of BPH generally occurs between the late 7th and mid 8th decade of life. This stage is characterized by an abrupt increase in the mass of the individual nodules, which may result in clinically significant BPH. Autopsy studies have demonstrated that approximately 50% of men have histologic evidence of BPH by age 60 and this percentage increases to 80% of men by age 80. At relatively young ages (less than 45 years), most men do not show any evidence of BPH.

BPH is the most common benign tumor in men and is an age related condition impacting significantly on the morbidity as well as the health care expenditures of the segment of the population that is over 65 years of age. The number of Medicare patients who have symptomatic BHP in 1990 equated to 4,996,000 (this figure does not represent the number of patients who were treated). Assuming an equal prevalence rate, changing population demographics will result in approximately 8,536,000 people over age 65 to suffer from symptomatic BPH by the year 2020.

There are a number of currently available treatments for BPH. These range from extremely invasive procedures such as open prostatectomy, transurethral incision of the prostate, transurethral resection of the prostate and laser prostatectomy. Other less invasive procedures include the application of electromagnetic energy in the form of microwaves or radio frequency waves, the application of high intensity focused ultrasound, the insertion of prostatic stents and prostatic balloon dilation. Currently there are several accepted pharmacological treatments including alpha blockade and androgen suppression. Notwithstanding the availability of treatment regimens, there still exists a need for a simple effective mechanism of treating this extremely prevalent condition. The present invention provides a mechanism to treat malignant tumors, both localized and metastatic, as well as benign hyperproliferative disorders and disorders resulting from a decrease in apoptosis, such as BPH, using vectors that deliver chemically inducible, apoptosis inducing factors into cells.

### SUMMARY OF THE INVENTION

It is an object of this invention to construct a conditionally lethal gene that can be activated to cause apoptosis in a cell in which it is present.

It is an object of this invention to provide a conditionally lethal, apoptosis inducing gene suitable for use in gene therapy applications involving non-proliferating cells.

It is an object of this invention to construct a conditionally lethal gene that is non-toxic unless induced.

It is an object of this invention to construct a conditionally lethal, apoptosis inducing gene that does not require membrane localization.

It is an object of this invention to construct a conditionally lethal, apoptosis inducing gene that bypasses endogenous apoptosis control mechanisms.

It is an object of this invention to provide a gene therapy treatment for selectively deleting specific cells.

It is an object of this invention to provide a gene therapy vector that expresses a chemically inducible apoptosis factor. The gene therapy vectors of the present invention may comprise viruses, plasmids or nucleic acids. In preferred embodiments, the gene therapy vector is a virus selected from the group consisting of adenoviruses, herpes viruses, pox viruses, retroviruses and adeno-associated viruses.

It is an object of this invention to provide a recombinant adenovirus expressing an apoptosis inducing, conditionally lethal gene.

It is an object of this invention to provide a treatment modality for treatment of both localized and metastatic tumors comprising a vector which delivers a chemically inducible, apoptosis inducing gene.

It is an object of this invention to provide a treatment modality for the treatment of benign proliferative disorders and disorders in which there is a loss of naturally occurring apoptosis, including but not limited to benign prostate hyperplasia and atherosclerosis, comprising the delivery of a vector expressing a chemically inducible, apoptosis inducing gene.

It is an object of this invention to provide transgenic animals that express chemically inducible apoptosis factors in specific cell types.

It is an object of this invention to provide a method of determining the biological role of a specific cell type.

These and other objects are accomplished by the construction of a recombinant, chemically inducible version of an intermediate factor involved in the apoptosis cascade. Since many of the events in signaling are regulated by protein-protein interactions, signaling intermediates, as exemplified by caspases, are ideal candidates for designing conditionally lethal alleles based on chemically-induced dimerization (CID) (13-15).

### BRIEF DESCRIPTION OF THE DRAWINGS

FIG. 1. Schematic representation of the Fas-mediated apoptosis cascade.

FIG. 2. Schematic representation of a conditional, chemically inducible Fas receptor.

FIG. 3. Schematic representation of the conditionally lethal genes of the present invention.

FIG. 4. Diagram showing the steps followed in constructing the recombinant molecules of the present invention.

FIG. 5. Graph of reporter enzyme activity secreted from cells transfected with various amounts of a caspase-1 construct.

FIG. 6. Graph of reporter enzyme activity secreted from cells transfected with various amounts of a caspase-1 construct.

FIG. 7. Graph of reporter enzyme activity secreted from cells transfected with various caspase-3 constructs.

FIG. 8. Graph of reporter enzyme activity secreted from cells transfected with various caspase-3 constructs.

FIG. 9. Graph of reporter enzyme activity secreted from cells transfected with various caspase-3 constructs.

FIG. 10. Western blot of cells transfected with various caspase constructs and probed with anti-HA antibody.

FIG. 11. Western blot of cells transfected with various caspase-3 constructs and probed with anti-HA antibody.

FIG. 12. Graph of reporter enzyme activity secreted from cells transfected with either a caspase-1 construct, a Bcl-x_{L} construct or co-transfected both caspase-1 and Bcl-x_{L} constructs.

FIG. 13. Graph of reporter enzyme activity secreted from cells transfected with either a caspase-3 construct, a Bcl-x_{L} construct or co-transfected both caspase-3 and Bcl-x_{L} constructs.

FIG. 14. Bar graph of reporter enzyme activity secreted from cells transfected with either a caspase-1 construct, a Bcl-x_{L} construct or co-transfected both caspase-1 and Bcl-x_{L} constructs and treated with an anti-Fas antibody before the assay was conducted.

FIG. 15. Bar graph of reporter enzyme activity secreted from cells transfected with either a caspase-3 construct, a Bcl-x_{L} construct or co-transfected both caspase-3 and Bcl-x_{L} constructs and treated with an anti-Fas antibody before the assay was conducted.

FIG. 16. Bar graph showing the percent survival of cells transfected with various constructs.

FIG. 17. Bar graph showing the sensitivity of different cell lines to conditionally lethal Fas, caspase-1 and caspase-3 constructs.

FIG. 18. Structures of the chemical inducers used.

FIG. 19. Panel A. Map of the plasmid used to construct a recombinant adeno expressing and ADS under the control of the CMV promoter. Panel B. Photograph of an agarose gel showing the results of a restriction digest analysis of the plasmid.

FIG. 20. Panel A. Map of a plasmid used to construct a recombinant adenovirus expressing an ADS under the control of the SRα promoter, Panel B. Photograph of an agarose gel showing results of a restriction digest analysis of the plasmid.

FIG. 21. Bar graph showing reporter enzyme activity in BPH derived CR smooth muscle cells treated with the constructs of the present invention.

FIG. 22. Bar graph showing reporter enzyme activity in BPH derived JD smooth muscle cells treated with the constructs of the present invention.

FIG. 23. Design of conditional Fas signaling intermediates. (A) Model of CID-regulated caspases. Transmembrane diffusion of CIDs (e.g. FK1012, AP1903) leads to the crosslinking of intracellular pro-caspases that are genetically fused to one or more CID-binding domains (e.g. FKBP12), leading to transproteolysis and processing to their fully active forms. The caspase active-site consensus sequence, QAC(R/Q)G, is shown. (B) Schematic of CID-regulated pro-apoptotic molecules showing the CID-binding domain (i.e. Fᵥ=FKBP12_{V36}), intracellular targeting sequences (i.e. M (myristoylation-targeting sequence); N (nuclear localization sequence); and Mas70₃₄ (mitochondria-targeting sequence)), pro-apoptotic molecules (i.e. Caspase 1, 3, and 8, Fas cytoplasmic domain (residues 179-319), and FADD₁₂₅ (death effector domain)), and hemaglutinin epitope tag (E).

FIG. 24. Activation of caspase-1 and -8, but not -3, by high-specificity CID, AP1903, requires a flexible linker between FKBP12 and caspase domains. (A-F) Jurkat-TAg cells were transiently transfected with 2 μg of reporter plasmid, SRα-SEAP, along with indicated amount of expression plasmid, pSH1, containing Fᵥ/caspase fusion proteins or control Fᵥs. After 24 hours cells were treated with half-log dilutions of AP1903 (or FK1012) and incubated for an additional 20 hours before extracts were assayed for SEAP activity. (A) Dimerization is sufficient for caspase-3 activation. Cells received 4 μg S-Casp3 (▴), S-Fᵥ1-Casp3 (□), S-Fᵥ2-Casp3 ( [symbol] ), or S-Fᵥ3-Casp3 (Δ). (B) Caspase-3 activation is not sterically hindered by amino terminal FKBP12. Cells received 4 μg S-Fᵥ1-Fᵥᵢₛ1 (●), S-Fᵥ1-Fᵥᵢₛ-Casp3 (◯), S-Fᵥᵢₛ2-Casp3 (▴) S-Fᵥ1-Casp3 (□), S-Fᵥ2-Casp3 ( [symbol] ), or S-Fᵥᵢₛ1-Casp3 (Δ). (C) Caspase-1 activation is sterically hindered by amino terminal FKBP12. Cells received 4 μg S-Fᵥ2-Casp3 (+AP1903 (□)), (+FK1012 ( [symbol] )), S-Fᵥ2-Casp1 (+AP1903(Δ), (+FK1012 (▴)), or S-Fᵥ1-Fᵥᵢₛ1 (+AP1903 (●). (D) A flexible linker confers AP1903-sensitivity to caspase 1. Cells received 2 μg (□), 1 μg ( [symbol] ), or 0.5 μg (Δ) S-Fᵥ1-Fᵥᵢₛ1-Casp1, or 4 μg control plasmid, S-Fᵥ1-Fᵥᵢₛ1 (▴). (E) A flexible linker confers AP1903-sensitivity to caspase 8. Cells received 4 μg (□), 2 μg ( [symbol] ), or 1 μg (Δ) S-Fᵥ1-Casp8, or 4 μg control plasmid, S-Fᵥ1-Fᵥᵢₛ1 (▴). (F) A single short G-S linker augments the AP1903-sensitivity of caspase 1. Cells received 1 μg S-Fᵥ1-Fᵥᵢₛ1-Casp-1 (Δ), S-Fᵥᵢₛ1-Casp1 (□), S-Fᵥᵢₛ2-Casp1 ( [symbol] ), or 4 μg S-Fᵥ1-Fᵥᵢₛ1 (▴). Inset (A and B) Equal aliquots of cell extracts were analyzed by western blot with MoAb to the HA epitope (“E” FIG. 24B).

FIG. 25. Crosslinking the death effector domain of FADD is sufficient for triggering apoptosis with reduced basal toxicity relative to Fas, caspase 1 and 8. (A-D) As above, Jurkat-TAg cells were transfected with 2 μg SRα-SEAP plus the indicated expression plasmids. (A) FADD₁₀₀ is sufficient for FK1012-mediated cytotoxicity. Cells received 4 μg S-Fₚₖ3-FADD₁₂₅ (□), S-Fₚₖ3-FADD₈₀ (Δ), S-Fₚₖ3-FADD₁₀₀ ( [symbol] ), or S-Fₚₖ3-Δ25FADD₁₂₅ (▴). Inset (A) Equal aliquots of cell extracts were analyzed by western blot as above. (B) Cells received 4 μg S-Fₚₖ3 (□), S-Fₚₖ3-FADD125V82 (▴), or S-Fₚₖ3-FADD₁₂₅ ( [symbol] ). (C) Fas, caspase 1, and caspase 8 have high basal activity relative to FADD₁₂₅ and caspase 3. (D) Caspase 1 is the most AP1903-sensitive ADS developed). (C and D) Cells received 2 μg S-Fᵥ1-Fᵥᵢₛ1 (□), S-Fᵥ2-Fas ( [symbol] ), S-Fᵥ1-Fᵥᵢₛ1-FADD₁₂₅ (Δ), S-Fᵥ1-Fᵥᵢₛ1-Casp8 (▴), S-Fᵥ1-Fᵥᵢₛ1-Casp1 (◯), or S-Fᵥ1-Fᵥᵢₛ1-Casp3 (●).

FIG. 26. Plasma membrane targeting of caspase-3 increases its AP1903 sensitivity and basal activity. (A-B) Transient transfection assay was performed as above. (A) Cells received 2 μg SRα-SEAP plus 4 μg N2-Fᵥ2-Casp3 (□), Mas70₃₄-Fᵥ2-Casp3 ( [symbol] ), or S-Fᵥ2-Casp3 (Δ), or 1 μg M-Fᵥ2-Casp3 (▴). (B) Cells received 2 μg SRα-SEAP plus 4 μg (□), 1 μg ( [symbol] ), or 0.25 μg (Δ) M-Fᵥ2-Casp3, or 4 μg S-Fᵥ2-Casp3 (▴), or 4 μg control plasmid, S-Fᵥ1-Fᵥᵢₛ1 (◯). (C-J) Localization of caspase-3 to different intracellular membranes. HeLa cells transiently transfected with cytoplasmic S-Fᵥ2-Casp3-E (C), plasma membrane-localized M-Fᵥ2-Casp3-E (D), mitochondria-localized Mas70₃₄-Fᵥ2-Casp3-E (E), or nuclear N2-Fᵥ2-Casp3-E (F) were fixed, stained with anti-HA antibodies and examined by confocal microscopy. Alternatively, control proteins were localized, including S-Fᵥ2-E (G), M-Fᵥ2-E (H), Bcl-x_{L}-E (I), or Gal4-VP16-E (J). In each case, cells shown are representative of several transfected cells.

FIG. 27. Nuclear-targeted caspase-1, -3, and -8 trigger apoptosis. (A-B) Transient transfection assay was performed as above. (A and B) Cells received 2 μg SRα-SEAP plus 2 μg nuclear targeted caspases, including N2-Fᵥ1-Fᵥᵢₛ1-Casp1 (Δ), N2-Fᵥ1-Fᵥᵢₛ1-Casp3 (▴), N2-Fᵥ1-Fᵥᵢₛ1-Casp8 ( [symbol] ), or control construct N2-Fᵥ1-Fᵥᵢₛ1 (□). (C) Nuclear targeted caspase-3 functions efficiently. Cells received SRα-SEAP plus 4 μg (□) or 1 μg (Δ) S-Fᵥ2-Casp3, 4 μg ( [symbol] ) or 1 μg (▴) N2-Fᵥ2-Casp, or 1 μg S-Fᵥ1-Fᵥᵢₛ1 (◯). (D) Nuclear targeted FADD₁₂₅ has reduced activity. Cells received reporter plasmid plus 2 μg S-Fᵥ1-Fᵥᵢₛ1-FADD₁₂₅ ( [symbol] ), N2-Fᵥ1-Fᵥᵢₛ1-FADD₁₂₅ (□), or N2-Fᵥ1-Fᵥᵢₛ1 (Δ).

FIG. 28. Outline of the Construction of pAdTrack-CMV-Fᵥᵢₛ1-Yama-E and pAdTrack-CMV-E-Fᵥ1-Fᵥᵢₛ1-ICEst.

FIG. 29. A) Representation of pADTrack-CMV; B) and C) Gel of miniprep check of pADTrack-CMV-Fᵥᵢₛ1-Yama-E.

FIG. 30. A) Representation of pADTrack-CMV; B) and C) Gel of miniprep check of pAdTrack-CMV-E-Fᵥ1-Fᵥᵢₛ1-ICEst.

FIG. 31. Representation of pSH1/S-E-Fᵥ1-Fᵥᵢₛ1-ICEst;

FIG. 32. Representation of pSH1/S-Fᵥᵢₛ1-Yama-E;

FIG. 33. Outline of Generation of ADV-GFP-CMV-Yama-E and ADV-GFP-CMV-E-ICE;

FIG. 34. Schematic diagram showing general protocol for generation of ADV-GFPCMV-Yama-E and ADV-GFP-CMV-E-ICE;

FIG. 35. Representation of pADEasy-1;

FIG. 36. Gel of miniprep checks of pADEasy-1-Track-CMV-E-ICE and pAdEasy-1-Track-CMV-Yama-E;

FIG. 37. A)-C) Outline of Construction of pShuttle-CMV-E-Fᵥ1-Fᵥᵢₛ1-ICEst and Generation of ADV-CMV-E-ICE;

FIG. 38. Schematic representation of protocol forADV-CMV-E-ICE;

FIG. 39. A) Representation of pShuttle-CMV and B) miniprep gel;

FIG. 40. Outline of Luciferase assay of pShuttle-CMV-Fᵥ1-Fᵥᵢₛ1-ICE-E and results;

FIG. 41. Outline of Assay of Effect of Ad-YAMA and Ad-ICE on Different cell types;

FIG. 42. Graph showing effect of Ad-YAMA and Ad-ICE on T-C2G cells;

FIG. 43. Graph showing effect of Ad-YAMA and Ad-ICE on T-C2 cells;

FIG. 44. Graph showing effect of Ad-YAMA and Ad-ICE on JD-2a cells;

FIG. 45. Graph showing effect of Ad-YAMA and Ad-ICE on LNCaP cells;

FIG. 46. Outline and Western Blot showing expression and activation of ICE and YAMA;

FIG. 47. A) untreated JD-2a cell culture; B) control culture incubated with Adv-Fv1-YAMA expressing green fluorescent protein; C) cell culture incubated with virus and maintained in 50 nM AP1903;

FIG. 48. Plated PC-3 cells incubated with ADV-FKBP/ICE and treated (+) or untreated with AP1903 at increasing MOI;

FIG. 49. Plated JD-2a BPH cells incubated with ADV-FKBP/ICE and treated (+) or untreated with AP1903 at increasing MOI;

FIG. 50. Diagram illustrating protocol for treatment of s.c. prostate adenocarcinoma in situ with CID inducible caspases;

FIG. 51. Results of treatment of s.c. prostate adenocarcinoma with ADV-FKBP/ICE;

FIG. 52. Results of treatment of s.c. prostate adenocarcinoma with ADV-FKBP/ICE followed by administration of CID.

### DETAILED DESCRIPTION OF THE PREFERRED EMBODIMENTS

The strategy employed in this study to develop sensitive, conditional cytotoxic molecules relies on chemically-induced dimerization (CID) (15-17), and the discovery that full-length apoptosis signal transducing molecules, such as caspase zymogens, can undergo inter-molecular processing to become fully active (reviewed in 20).

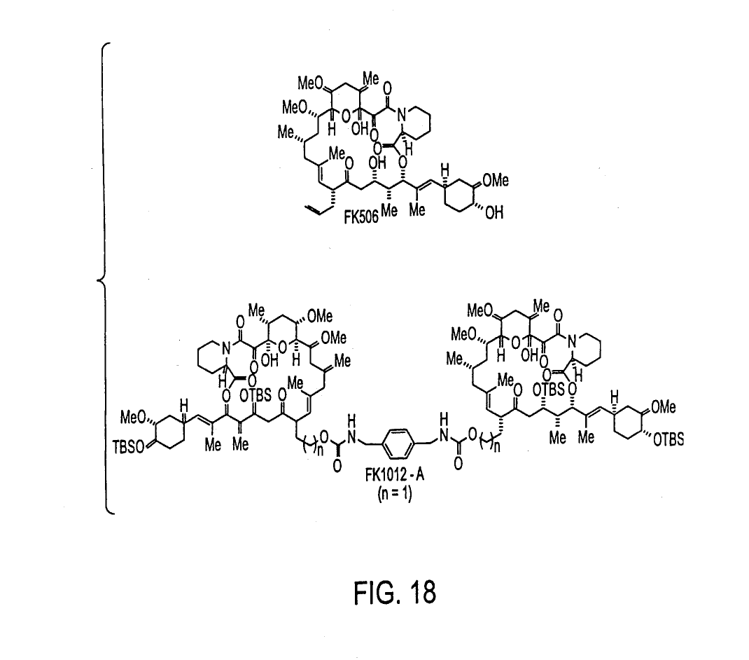

A chemical inducer of dimerization (CID) is defined as a dimer of the ligand for a CID-binding domain (CBD). Responsiveness to CIDs is achieved by fusing CBDs to target proteins. In this manner, CID administration leads to protein crosslinking. The CIDs used in this study include the previously described, non-immunosuppressive FK506 dimer, FK1012, and a novel fully synthetic FK1012 analog, AP1903, that is analogous to FK1012 acetylated at the C₉ position of the FK506 moieties (FIG. 18). This modification of FK506 prevents binding of AP1903 to the highly-expressed endogenous FKBPs (Kₘ FKBP12≈250 Nm), but leads to sub-nanomolar affinity to a mutant FKBP12 (F36V, abbr. Fᵥ) (M. Gilman, personal communication). The valine substitution of Fᵥ creates a deeper drug-binding pocket, which accommodates the acetyl groups of AP1903.

Specific examples of conditionally lethal molecules comprising a chemical inducer binding domain fused to an apoptosis signal transducing factor described herein are conditional alleles of the zymogens caspase-1 and caspase-3. The CID-binding domain (CBD), FKBP12, has been placed at the amino terminus of these proteins adjacent to the pro-domains of the inactive proteases. Upon administration of a lipid-permeable dimerizing drug, aggregation of apoptosis signal transducing factors, in this case caspases, occurs, leading to auto-proteolysis and activation. Further, it is demonstrated that this chemical activation of either caspase-1 or caspase-3 is sufficient to trigger apoptosis in target cells. This technique is termed chemically-induced apoptosis (CIA) and the recombinant molecules are termed artificial death switches (ADS) or chemically inducible apoptosis factors.

Although conditional caspase-1 alleles are somewhat autotoxic, like previously described conditional Fas alleles (5), the conditional caspase-3 alleles appear to be completely non-toxic in the absence of CID, even at high levels of expression. Interestingly, a truncated caspase-3 lacking its pro-domain is somewhat autotoxic, consistent with other reports that the pro-domains of caspases contribute to maintaining their quiescence in unstimulated cells (16). Further, the conditional caspase-1 allele appears to be completely insensitive to excess Bcl-x_{L} while the conditional caspase-3 allele can be blocked by an excess of Bcl-x_{L} levels. Finally, both conditional caspase-1 and caspase-3 alleles can trigger apoptosis in a broad range of tissues. These results confirm that crosslinking caspases can lead to their activation in intact cells and demonstrate an expanded repertoire of proteins that can be activated by CID.

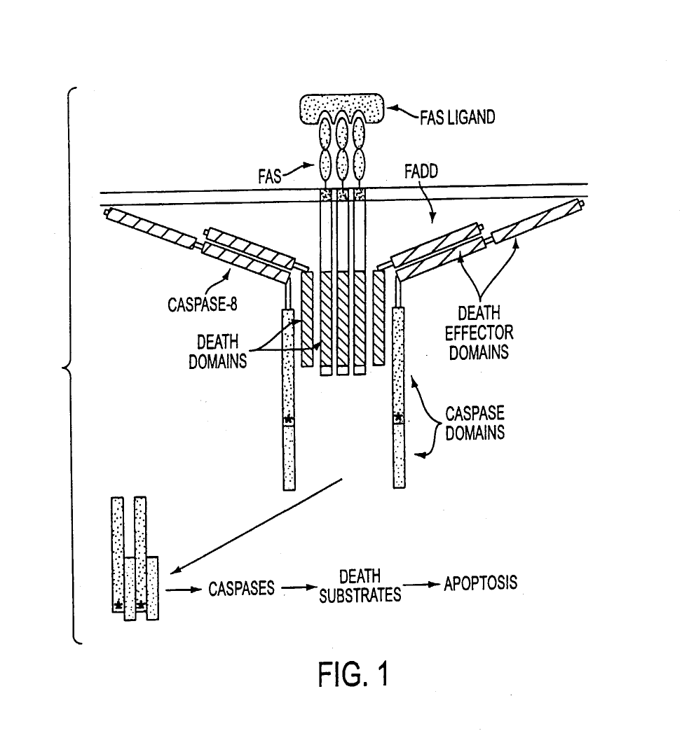

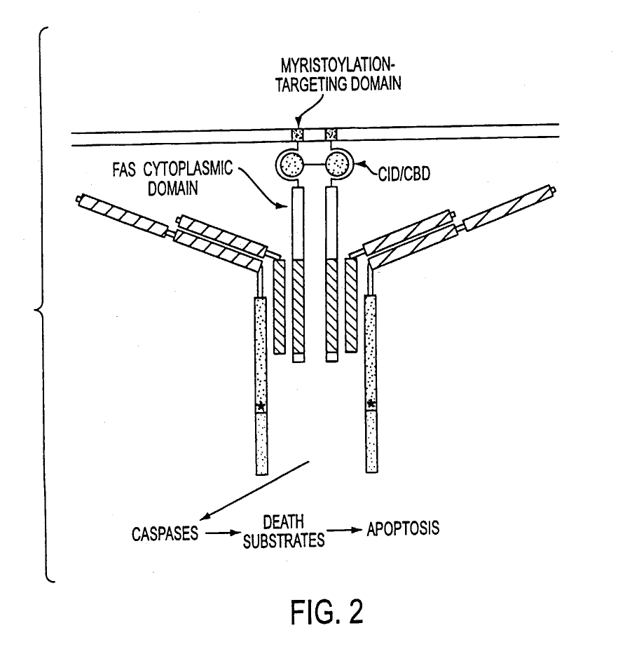

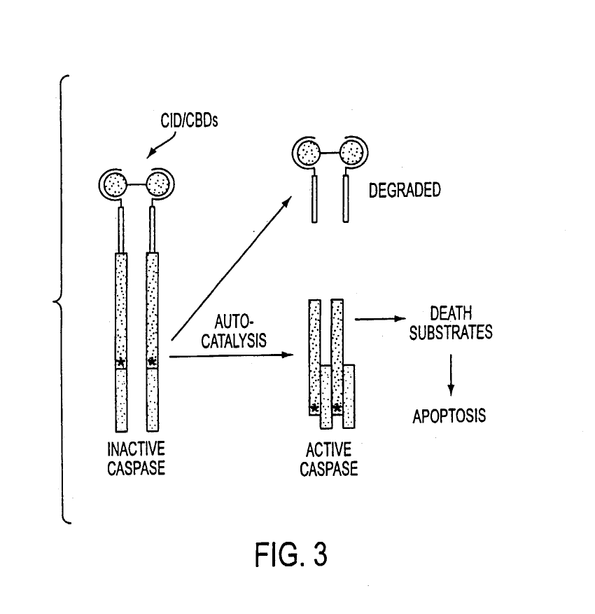

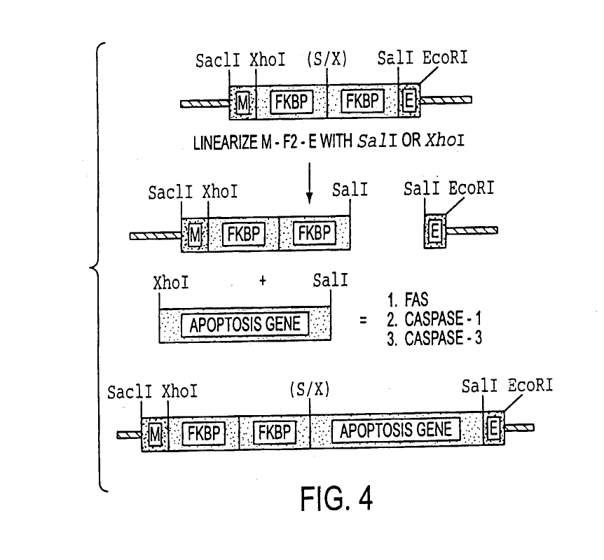

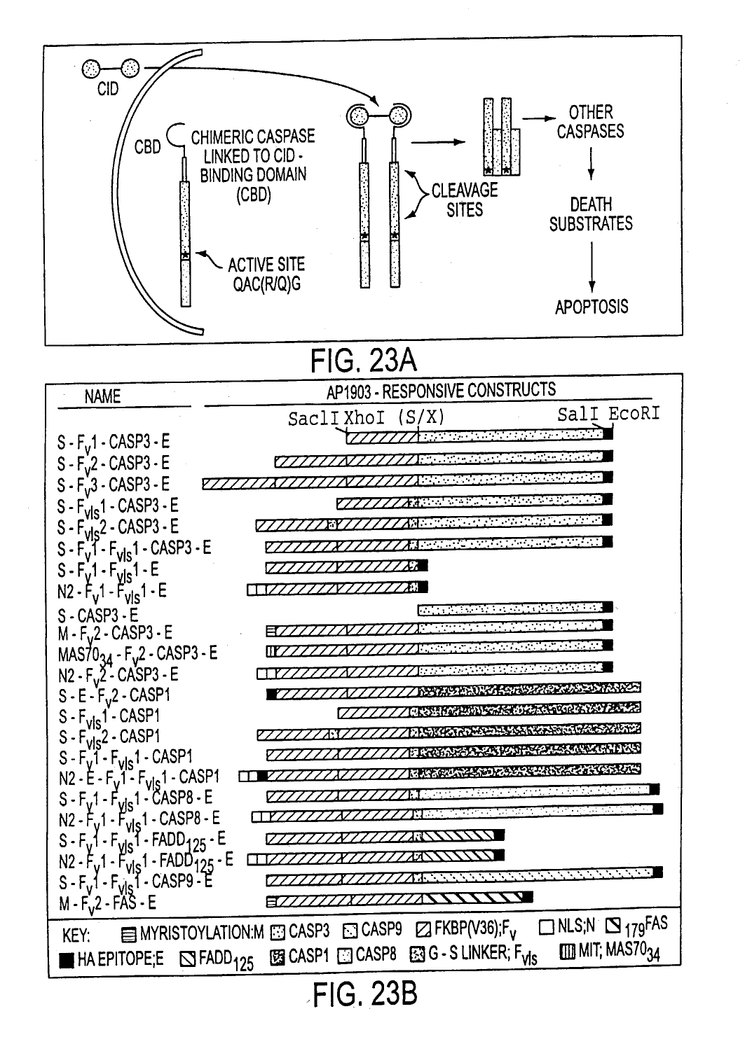

The pro-apoptotic conditional alleles were designed based on currently accepted models of Fas signaling (FIG. 1). In the design of conditional Fas (FIG. 2), FKBP12 (or mutants) replaces the extracellular domain of Fas, and the myristoylation-targeting domain (M) of c-Src (residues 1-14) directs membrane localization (5). For conditional caspases (FIGS. 3 and 23), CBDs are attached to their pro-domains. Following CID, pro-domains should be cleaved and the resulting, fully active proteases should be indistinguishable from wild-type proteins. All constructs use in connection with Examples 2 through 8 follow the cassette cloning strategy outlined in FIG. 4.

Caspase activation is a common integration point for diverse apoptotic stimuli and is therefore a logical control point for CIA. Given the examples of the instant invention, it will be possible to use other factors involved in the signal transduction of apoptotic stimuli as chemically inducible apoptosis factors. Chimeric molecules containing a chemically inducible dimerization domain fused to the precursor form of the signal transducing molecule can be readily constructed by those of skill in the art. The present invention envisions the use of such molecules in a fashion entirely analogous to the caspase examples set forth below.

Examples of suitable apoptotic stimuli transducing molecules include, but are not limited to, receptors such as: the tumor necrosis factor family receptors, such as TNFRI (p55) and TNFRII (receptor 2, p75); DR3; DR4 (TRAIL-R1); DR5 (TRAIL-R2); TRAIL-R3; CD30; CD27; and p75NTR (neurotrophin receptor). Other suitable molecules include adapter molecules such as FADD, TRADD, RAIDD, Casper, SIVA, DAXX, MADD and the like. Additional molecules that may be used to practice the present invention include all other members of the caspase family and apoptosis related serine/threonine kinases such as JNK1,2,3 and p38_{α,β,γ}. Another class of molecules that may be used to practice the present invention are Bcl-2 family members that trigger apoptosis such as: Bax; Bak; BAD; Bcl-xₛ; BIK, HRK, Bid, Bim and the like. Constructs comprising proteases, such as calpain, and constructs comprising sphingomyelinases, neutral and acid, may also be used to practice the present invention.

### EXAMPLE 1

General experimental methods for examples 2-8.

Plasmid construction.

To make M-Fₚₖ2, Fₚₖ3, and Fᵥ2, Fₚₖ (hFKBP12 (P89,K90)) and Fᵥ (HFKBP12(V36)) were amplified by PfuI PCR using primers: 5′-GCGACA CTCGAG GGA GTG CAG GTG GAA ACC-3′ (SEQ ID NO:1) and 5′-CGACA GTCGAC TTC CAG TTT TAG AAG C-3′ (SEQ ID NO:2) and the Fₚₖ template, HFKBP(P89,K90) (17) or the Fᵥ template, M46. The resulting products (and all other PCR fragments) were blunt-end ligated into EcoRV-digested Pbluescript (Stratagene) to create PKS/Fₚₖ and PKS/Fᵥ and sequenced. The 330 bp Xho1/Sal1 fragments from PKS/Fₚₖ and PKS/Fᵥ were ligated in tandem into Xho1/Sal1-digested MF3E and SF1E (described previously in 18) to make Fₚₖ3-E (three copies of Fₚₖ), M-Fₚₖ2-E (two copies Fₚₖ) and Fᵥ2-E (two copies Fᵥ). An additional 5′-epitope (E) was added to Fₚₖ3-E to produce E-Fₚₖ3-E by cloning hybridized oligonucleotides 5′-TCGAC TAT CCG TAC GAC GTC CCA GAC TAC GCA C-3′ (SEQ ID NO:3) and 5′-TCGAG TGC GTA GTC TGG GAC GTC GTA CGG ATA G-3′ (SEQ ID NO:4) into the 5′ XhoI site. M-Fₚₖ2-Fas was constructed by subcloning the Xho1/Sal1-Fas fragment from PKS/Fas (described previously 5) into Sal1-digested M-Fₚₖ2-E vector. Caspase-1, caspase-3 and Δ20caspase-3 inserts were PCR amplified from plasmids pCDNA3/hICE/AU1 and PCDNA3/YAMA using the following primers containing Xho1 sites (5′) and Sal1 sites (3′): HICE5X, 5′-CCGACA CTCGAG GCC GAC AAG GTC CTG AAG GAG-3′ (SEQ ID NO:5); HICE3′S, 5′-CGTAGA GTCGAC GTC CTG GGA AGA GGT AGA AAC-3′ (SEQ ID NO:6); YAMA5X, 5′-CCGACA CTCGAG GAG AAC ACT GAA AAC TCA GTG-3′ (SEQ ID NO:7); YAMA3S, 5′-CGTAGA GTCGAC GTG ATA AAA ATA GAG TTC TTT TGT-3′ (SEQ ID NO:8); 20Yam5x, 5′-ACA CTCGAG ATA CAT GGA AGC GAA TCA ATG G-3′ (SEQ ID NO:9). PCR products were subcloned into Pbluescript to create pKS/ICE, pKS/YAMA and pKS/20YAMA. Xho1/Sal1 fragments from these plasmids were then ligated into Sal1-digested E-Fₚₖ3-E (abr. Fₚₖ3) and Fᵥ2-E vectors, to produce Fₚₖ3-casp-1, Fᵥ2-casp-3, Fₚₖ3-casp-3, and Fₚₖ3-20casp-3. To make casp-3/S163, the 340 bp StuI-SalI fragment of pKS/YAMA was reamplified using primers 5′-ATT CAG GCC TCC CGT GGT ACC GAA CTG GAC TGT GGC ATT GAG-3′ (SEQ ID NO:10) and YAMA3S, subcloned into pBluescript to make pKS/YAMAS and sequenced. The mutant StuI-SalI fragment was substituted with the wild-type fragment in pKS/YAMA to make pKS/YAMA/S136 and ultimately Fₚₖ3-casp-3/S136 and Fᵥ2-casp-3/S136. The SRα-SEAP reporter plasmid was created by cloning the secreted alkaline phosphatase (SEAP) cDNA from NFAT-SX into the polylinker of pBJ5 (13). Bcl-x_{L} was amplified from a Bcl-x_{L} cDNA using primers 5′-CCGACA CTCGAG TCT CAG AGC AAC CGG GAG CTG G-3′ (SEQ ID NO:11) and 5′-CGTAGA GTCGAC TTT CCG ACT GAA GAG TGA GCC CA-3′ (SEQ ID NO:12) and subcloned into XhoI/SalI-digested F1-E. All plasmids were prepared by two rounds of CsCl centrifugation. Underlined=restriction sites.

Tissue Culture.

Jurkat-TAg cells (19) were grown in RPMI 1640 medium, 10% Fetal Bovine Serum (FBS), 10 mM Hepes (pH 7.4), 100 units/ml penicillin, and 100 μg/ml streptomycin. HeLa and 293 cells were grown in Dulbeccos Modified Eagle Medium, 10% FBS and antibiotics.

SEAP Assays.

Jurkat TAg cells (10⁷) in log phase growth were electroporated (950 μF, 250V; Gene Pulser II) with expression plasmid and 1-2 μg SRα-SEAP. After 24 hours, transformed cells were stimulated with CID or anti-Fas antibody (CH.11, Kamiya Biomedical). After an additional 20 hours, supernatants were assayed for SEAP activity as described previously (15). Units of SEAP activity are reported directly and as a percentage of activity relative to no stimulation within the same transfections (% Relative Activity).

Western Blot Analysis.

Jurkat TAg cells were electroporated with 2 μg of plasmid, cultured 36 hours, and stimulated with drug for the indicated time period. Approximately 5×10⁵ cells were lysed in 100 μl RIPA buffer (0.01M TrisHCl pH 8.0, 140 mM NaCl, 1% Triton X-100, 1 mM phenylmethylsulfonyl fluoride, 1% sodium deoxycholate, 0.1% SDS) on ice for 30 minutes. Cell debris were pelleted and supernatants were boiled in 1:1 Laemmli sample buffer (with 5% β-ME) for 5 minutes. Equal volumes of extracts were separated on a 15% SDS-PAGE gel. Membranes were incubated with monoclonal anti-HA epitope antibody, HA.11 (BABCO) followed by polyclonal HRP-conjugated goat anti-mouse antibody (Bio-Rad). Bands were detected with SuperSignal-Chemiluminescent Substrate (Pierce).

Lipofection and Cell Selection.

Jurkat TAg cells (4×10⁶) were transfected with 12 μl DMRIE-C reagent (Gibco-BRL), 2 μg of green fluorescent protein (GFP) expression vector pEGFP (Clontech), 2 μg of the pMACS-H2-K^{k} vector (Miltenyi), and 2-4 μg of CID-responsive plasmids in OPTI-MEM I reduced serum media. Transfected cells were purified to approximately 60% by MACSelect magnetic bead selection on MiniMACS separation columns (Miltenyi) as assessed by FACS analysis (described below). Selected cells were resuspended in 2 ml Jurkat TAg media, split into two aliquots, and one was treated with drug.

Flow Cytometry.

Cells were washed with PBS+1% FBS, resuspended in staining buffer (PBS, 1% FBS, 0.5 μg/ml Propidium Iodide (PI)) and analyzed within 1 hour. Two color flow cytometry of PI (band pass=525) and GFP (band pass=620), was performed using a Coulter Epics XL MCL cytometer. Greater than 20,000 events were counted per sample. Gates were set using mock transfected cells, unstained cells transfected with GFP, and stained nontransfected cells. Data is reported as the percent of PI⁻/GFP⁺ cells after drug addition relative to untreated cells in triplicate cultures.

FuGENE™6 Transfection and Luciferase Assays.

HeLa and 293 cells were plated at 2×10⁵ cells/35 mm dish 18 hours before transfection. Plated cells were transfected with 2 μg of constitutive luciferase reporter pGL2-Control (Promega), 2 μg of test plasmid, and 6 μl FuGENE™6 (Boehringer-Mannheim) in OPTI-MEM I media. Jurkat TAg cells (4×10⁶) were transfected as described above with 2 μg of pGL2-Control, 2 μg of test plasmid, and 12 μl of DMRIE-C in OPTI-MEM I media. After 24 hours, cells were split into two groups and one was treated with 500 nM AP1903 (Fᵥ constructs) or FK1012 (Fₚₖ). After an additional 24 hours, cells were lysed in 100 μl of Reporter Lysis Buffer (Promega) with three freeze/thaw cycles and 10 μl of the supernatants (or constant dilutions) were assayed with 90 μl of Luciferase Assay Substrate (Promega) using a Turner TD-20e luminometer. Data is reported as the % of luciferase activity after drug addition relative to luciferase activity without drug in duplicate cultures. All transfections were performed at least three times and averaged.

### EXAMPLE 2

Crosslinking caspase-1 triggers apoptosis in mammalian cells.

Two critical factors that are required for a broadly applicable ADS are high sensitivity to drug and low basal toxicity. In previously reported experiments, it was demonstrated that a Fas-based ADS could trigger apoptosis in mammalian cells following FK1012 administration (5). However, overexpression of this Fas allele was somewhat autotoxic, consistent with reports that the cytoplasmic domain (DD) of Fas can spontaneously multimerize when expressed at high levels (21). The problem of autotoxicity was exacerbated by the relatively high expression level of chimeric M-FKBP₂-Fas required to kill cells efficiently. The requirement for a high level of expression of the chimeric Fas molecules was likely a result of the fact that FK1012 binds equally well to endogenous and ectopic FKBPs thus, a high concentration of recombinant Fas molecules was required to compete with the endogenous FKBPs.

An additional factor that may have contributed to the requirement for a high level of recombinant Fas expression to effect efficient cell killing is the presence of regulatory mechanisms designed to prevent the entry of the cell into apoptosis. Fas signaling does not always lead to apoptosis due to intracellular checkpoint genes, like Bcl-2 and Bcl-x_{L} (34,35), regulation of caspase-8 activation (22) or other mechanisms (23). Therefore, downstream effectors of apoptosis, such as caspase-1 or caspase-3 were constructed.

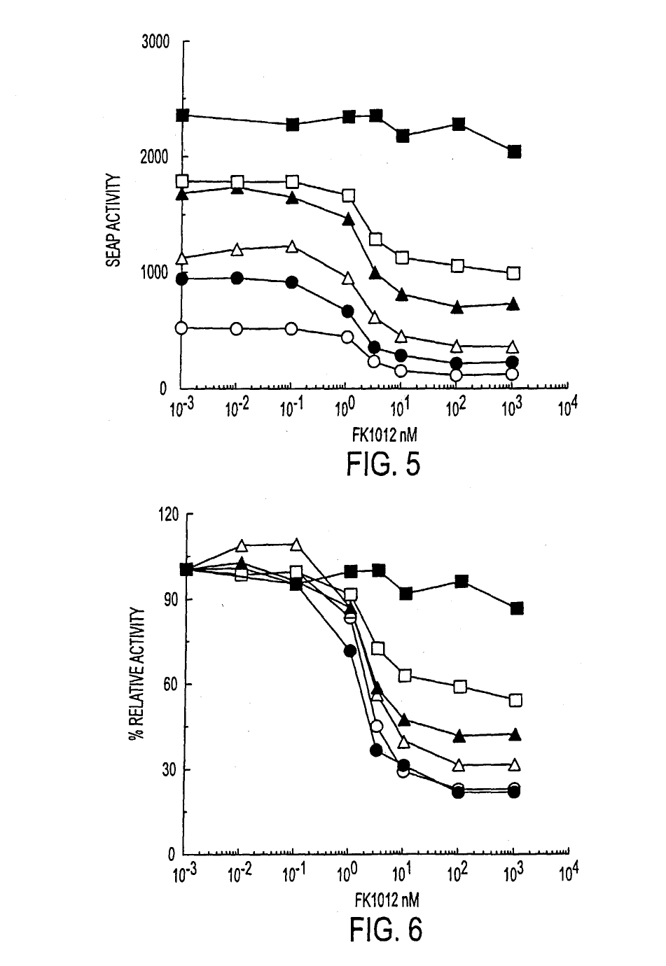

Jurkat TAg cells were cotransfected with reporter plasmid, SRα-SEAP, constitutively expressing secreted alkaline phosphatase, along with various amounts of a fusion construct in which caspase-1 is fused to 3 FKBP12s, Fₚₖ3-casp-1, or a control plasmid expressing 3 FKBPs, Fₚₖ3(FIGS. 5 and 6). Jurkat TAg cells were transiently-transfected with: 4 μg Fₚₖ3-casp-1 (open circles), 2 μg Fₚₖ3-casp-1 (closed circles), 1 μg Fₚₖ3-casp-1 (open triangles), 0.5 μg Fₚₖ3-casp-1 (closed triangles), 0.25 μg Fₚₖ3-casp-1 (open squares), or 4 μg Fₚₖ3 (closed squares). After 20 hours, transfected cells were treated with FK1012. After an additional 24 hours, SEAP activity was assayed and reported directly (FIG. 5) or as a percentage of activity from untreated cells in identical aliquots from the same transfections (FIG. 6). Data is representative of three independent experiments performed in duplicate. Apoptosis of cells is indicated by a net reduction in reporter activity.

The FKBP12 variant, Fₚₖ (P89,K90) binds FK1012 as well as wild-type FKBP12, but the two amino acid changes prevent CID-independent interactions with cellular proteins, like calcineurin (17). This eliminates a mild toxicity associated with overexpressing wild-type FKBP12. Similar to M-FKBP₃-Fas, in the presence of FK1012, there is a dramatic reduction of reporter activity in cells expressing Fₚₖ3-casp-1 that is not present in cells expressing control Fₚₖ3 (FIGS. 5 and 6). Also like conditional Fas, caspase-1 is autotoxic as indicated by the CID-independent reduction in reporter activity in the cells transfected with Fₚₖ3-casp-1 expressing plasmid (FIG. 5, compare open circles to closed squares). However, this autotoxicity can be greatly reduced with only a marginal decrease in efficacy by transfecting less caspase-1 plasmid (FIGS. 5 and 6, open squares).

### EXAMPLE 3

Conditional caspase-3 displays extremely low basal activity vet triggers apoptosis efficiently in the presence of CIDs.

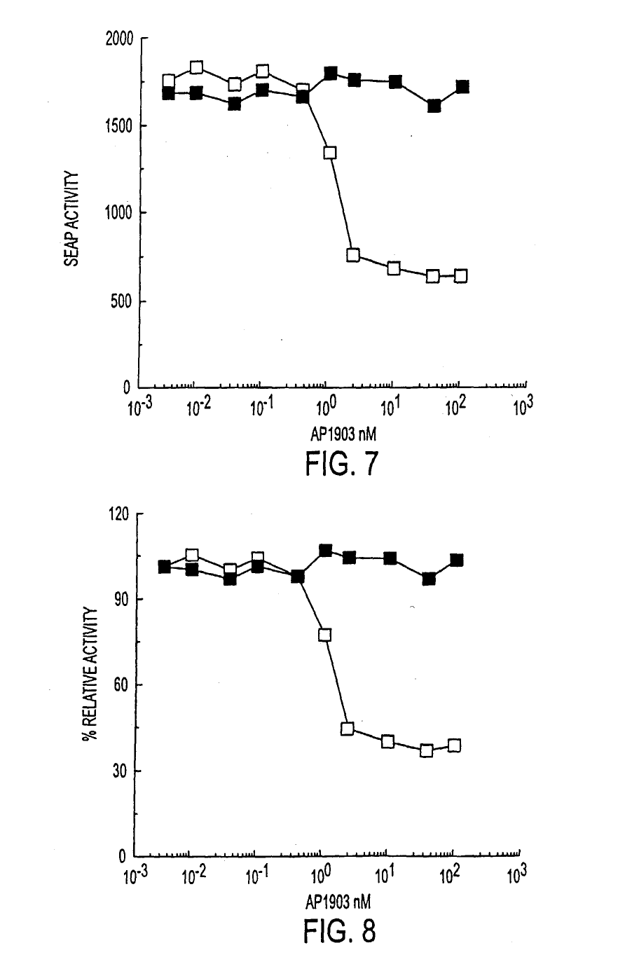

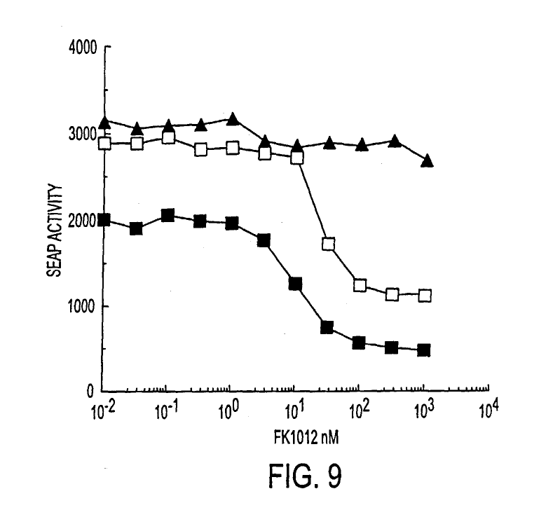

In contrast to caspase-1 and Fas, conditional caspase-3 alleles are not apparently autotoxic (FIGS. 7-9). With reference to FIGS. 7 and 8, Jurkat TAg cells were transiently-transfected with: 2 μg Fᵥ2-casp-3 (open squares), or 2 μg Fᵥ2-casp-3/S163 (closed squares). With reference to FIG. 9, Jurkat TAg cells were transfected with: 2 μg Fₚₖ3-casp-3/S163 (closed triangles), 2 μg Fₚₖ3-casp-3 (open squares), or 2 μg Fₚₖ3-Δ20casp-3 (closed squares). After 20 hours, transfected cells were treated and analyzed as before. Data is representative of at least three independent experiments performed in duplicate.

In the absence of the novel CID, AP1903, reporter activity is the same in cells transfected with Fᵥ2-casp-3, control Fᵥ2 (not shown) or control Fᵥ2-casp-3/S163, which is inactive due to the substitution of serine for cysteine within the conserved active site QACRG (SEQ ID NO:13) motif (FIGS. 7 and 8) (20). However, in the presence of AP1903 (EC₅₀≈1 nM), crosslinking caspase-3 was sufficient to trigger a dramatic reduction in reporter activity of ≈65%, comparable to Fas or caspase-1 signaling (FIG. 8). Enzymatically inactive caspase-3, Fᵥ2-casp-3/S163, could not reduce reporter activity even in the presence of 100 nM AP1903. The absence of toxicity of Fᵥ2-casp-3 (and Fₚₖ3-casp-3, FIG. 9) suggests that caspase-3 is less likely than caspase-1 to be spontaneously activated in Jurkat cells. The increased specificity of AP1903 for Fᵥ is reflected by an increased drug efficacy (compare FIGS. 7 and 9).

To test whether the pro-domain of caspase-3 contributes to low basal activity, 20 residues from the amino-terminus of caspase-3, comprising most of the pro-domain were removed. Interestingly, removal of the pro-domain makes caspase-3 somewhat autotoxic (FIG. 9, closed squares), although CID induces a more complete diminution of reporter.

### EXAMPLE 4

Administration of CID causes rapid processing of Fᵥ2-caspase-3.

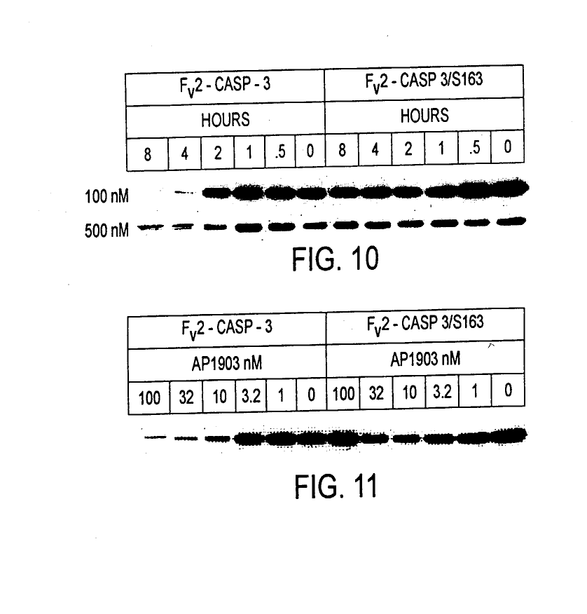

Processing of caspase-3, and other caspases, ordinarily involves two or more cleavages after aspartic acid residues. To confirm that the CID-mediated diminution of reporter activity was caused by activation of the conditional caspases, the processing and degradation of conditional caspase-3 was directly examined. Jurkat cells were transfected 2 μg Fᵥ2-casp-3 or Fᵥ2-casp-3/S163. After 36 hours, aliquots of cells were treated with 100 nM or 500 nM AP1903 for various times. Following these incubations, cell extracts were analyzed by western blotting using monoclonal antibodies to an epitope tag (HA) placed at both ends of the construct. The results are shown in FIG. 10. When 500 nM AP1903 was administered, degradation of full-length caspase-3 was complete within 2 hours, while no decrease in control caspase-3/S163 was detectable. When cells were treated with 100 nM AP1903, full degradation of Fᵥ2-casp-3 took four hours.

FIG. 11 shows an anti-HA epitope immunoblot of extracts from Jurkat TAg cells transfected as in FIG. 10 and treated for eight hours with half-log dilutions of AP1903 at the concentrations indicated. As little as 10 nM AP1903 was sufficient to cause the processing and degradation of the majority of Fᵥ2-casp-3. The breakdown products of caspase-3 activation were not seen even though epitope tags were present at both ends of the protein. Similar results were seen with Fₚₖ3-casp-1 (not shown) although a reduced amount of chimeric protein was seen, presumably due to autoprocessing of caspase-1. Therefore, homomultimerization of the caspases, caspase-1 and caspase-3, is sufficient for their activation.

### EXAMPLE 5

Conditional caspase-1 and caspase-3 trigger apoptosis in the presence of excess Bcl-x_{L}.

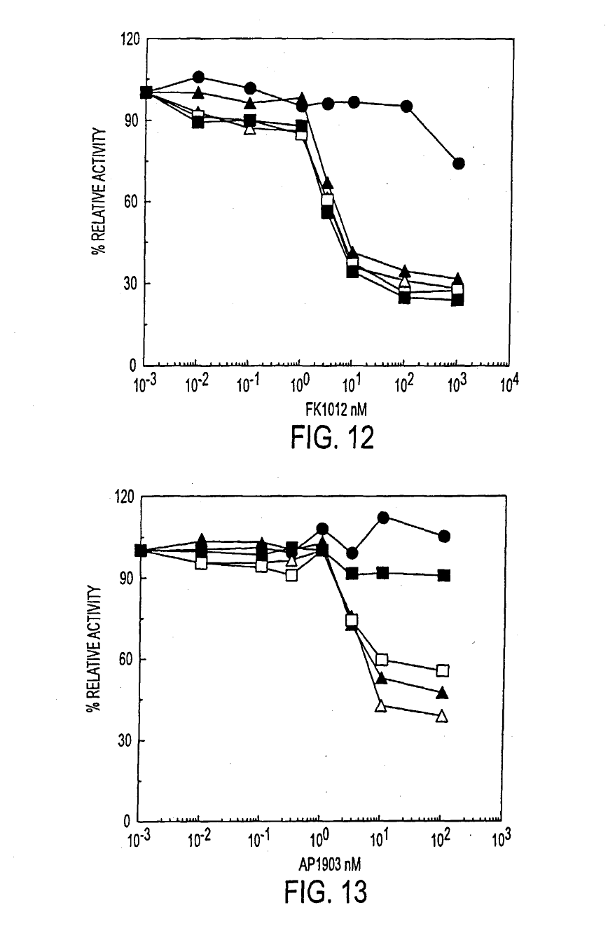

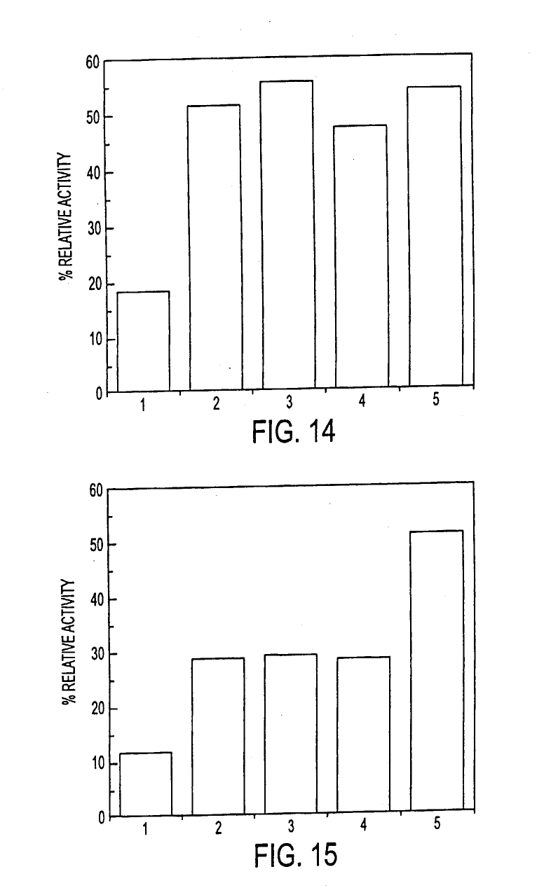

To determine if conditional caspases can bypass inhibition by the checkpoint gene Bcl-x_{L}, conditional caspase-1 and caspase-3 alleles were coexpressed along with an excess of Bcl-x_{L}. A comparison was made between the extent of apoptosis induced by chemical inducer (FIGS. 12 and 13) and the extent of apoptosis induced by anti-Fas antibody (FIGS. 14 and 15). The results of the caspase-1 alleles are presented in FIGS. 12 and 14 while the results obtained with caspase-3 alleles are presented in FIGS. 13 and 15.

Jurkat TAg cells were transiently transfected with the following caspase-1 construct expressing plasmids: Fₚₖ3-casp-1 (open triangles in FIG. 12; 1 in FIG. 14), Fₚₖ3-casp-1+1 μg Bcl-x_{L} (closed triangles in FIG. 12; 2 in FIG. 14),+2 μg Bcl-x_{L} (open squares in FIG. 12; 3 in FIG. 14), +4 μg Bcl-x_{L} (closed squares in FIG. 14; 4 in FIG. 14), or Fₚₖ3+4 μg Bcl-x_{L} (closed circles; 5 in FIG. 14). Jurkat TAg cells were transiently transfected with the following caspase-3 construct expressing plasmids: Fᵥ2-casp-3 (open triangles in FIG. 13; 1 in FIG. 15), Fᵥ2-casp-3+1 μg Bcl-x_{L} (closed triangles in FIG. 13; 2 in FIG. 15), +2 μg Bcl-x_{L} (open squares in FIG. 13; 3 in FIG. 15), +4 μg Bcl-x_{L} (closed squares in FIG. 13; 4 in FIG. 15), or Fᵥ2-casp-3/S163+4 μg Bcl-x_{L} (closed circles in FIG. 13; 5 in FIG. 15). Cells were transfected and assayed as before. Data is representative of at least three independent experiments. Data is given relative to untreated cells from the same transfection.

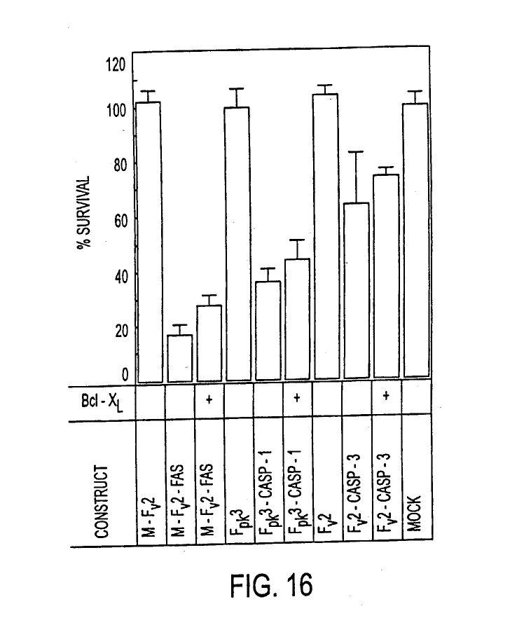

Although ectopically-expressed Bcl-x_{L} inhibits Fas-mediated apoptosis by ≈50% in Jurkat cells (FIG. 14 lanes 2-5), it consistently had no inhibitory effect on Fₚₖ3-casp-1 mediated apoptosis (FIGS. 12 and 16), even when a large molar excess of Bcl-x_{L} was expressed (FIG. 12). However, Bcl-x_{L} is able to block caspase-3 mediated signaling at a similar 4-fold molar excess (FIG. 13, closed squares). Likewise, the presence of Fᵥ2-casp-3 is able to reduce the inhibition of Fas signaling by Bcl-x_{L} by about 50% (FIG. 15, lanes 2-4). Control Fᵥ2-casp-3/S163 does not attenuate the protein function of Bcl-x_{L}, suggesting that a functional protease domain is necessary for the partial neutralization of Bcl-x_{L} (compare FIG. 15, lanes 2-5).

### EXAMPLE 6

Direct demonstration of apoptosis in Jurkat cells.

Since the above experiments are based on an indirect reporter assays for apoptosis and cannot definitively rule out that conditional molecules are reducing transcription, translation or protein stability of reporter, the efficacy of CIA was reexamined more directly. To enrich for ADS-expressing Jurkat cells, cells receiving conditional alleles were cotransfected with a selectable surface marker, consisting of the extracellular domain of the murine MHC molecule, K^{k}, and GFP to mark transfected cells and the indicated plasmids. 24 hours later, cells were sorted using magnetic bead-conjugated K^{k} antibodies, leading to an enrichment of 60% GFP⁺ cells from ≈15% unsorted. Sorted cells were split into two groups, one of which was treated with 500 nM AP1903 (Fᵥ chimeras) or FK1012 (Fₚₖ chimeras). After 24 hours, cells were stained with propidium iodide and analyzed by FACS to determine the percentage of viable GFP⁺/PI⁻cells. The % survival indicated is the percentage of viable cells after treatment with drug relative to the untreated aliquots. Also, in separate transfections, a 1:1 ratio of Bcl-x_{L}-containing plasmid to ADS-containing plasmid was added. Cells were analyzed by FACS to determine the percentage of GFP⁺ cells that survived after treatment with drug. While Bcl-x_{L} could inhibit endogenous Fas signaling by ≈50% (FIG. 14), there was very little Bcl-x_{L} effect on CID-mediated apoptosis by conditional caspase-1 or caspase-3 (FIG. 16). This demonstrates that conditional caspases can bypass intracellular checkpoints, including Bcl-x_{L}, and should therefore have broader usefulness than a Fas-based ADS.

### EXAMPLE 7

Conditional Fas, caspase-1 and caspase-3 trigger apoptosis in a wide panel of cell lines.

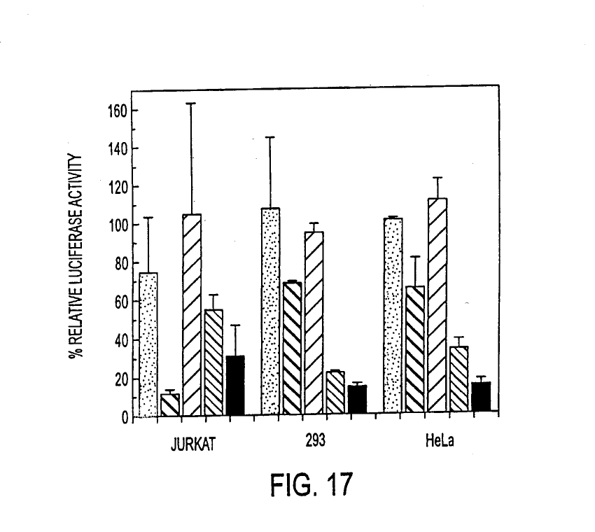

To determine if the conditional caspase-1 and caspase-3 alleles functioned in a range of cells, CID-mediated apoptosis was examined in various cell lines and the results are presented in FIG. 17. Jurkat TAg, 293 and HeLa cells were transiently-transfected with a constitutively-expressing luciferase reporter plasmid and control vector MFᵥ2 (speckled), MFᵥ2-Fas plasmid (wide stripe), control vector Fₚₖ3 (narrow stripe), Fₚₖ3-casp-1 (bricks), or Fᵥ2-casp-3 (solid). After 24 hours, transfected cells were split into duplicate cultures and 500 nM drug (AP1903 for Fᵥ, FK1012 for Fₚₖ) was added to one culture for an additional 24 hours. The % relative reporter activity is the percent of luciferase activity after drug addition relative to untreated cells. Error bars represent the standard deviation of the mean activity of three independent transfections. While conditional Fas did not trigger apoptosis in 293 cells, conditional caspase-1 and caspase-3 functioned in every cell tested. In addition to the cell lines reported here, high efficiency of killing has been demonstrated in the prostate cancer cell lines RM-1, RM-9, AND TRAMP-C2. In view of the foregoing, the caspase-based ADSs may lead to a more universally-applicable death switch.

### EXAMPLE 8

Conditional Fas and caspase-3 trigger apoptosis in smooth muscle cells derived from BPH.

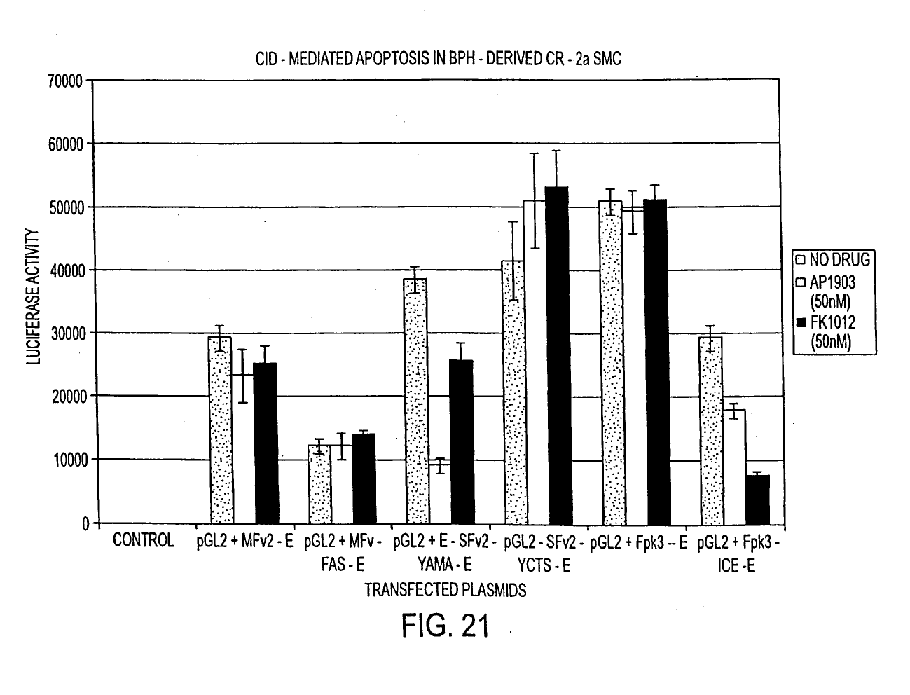

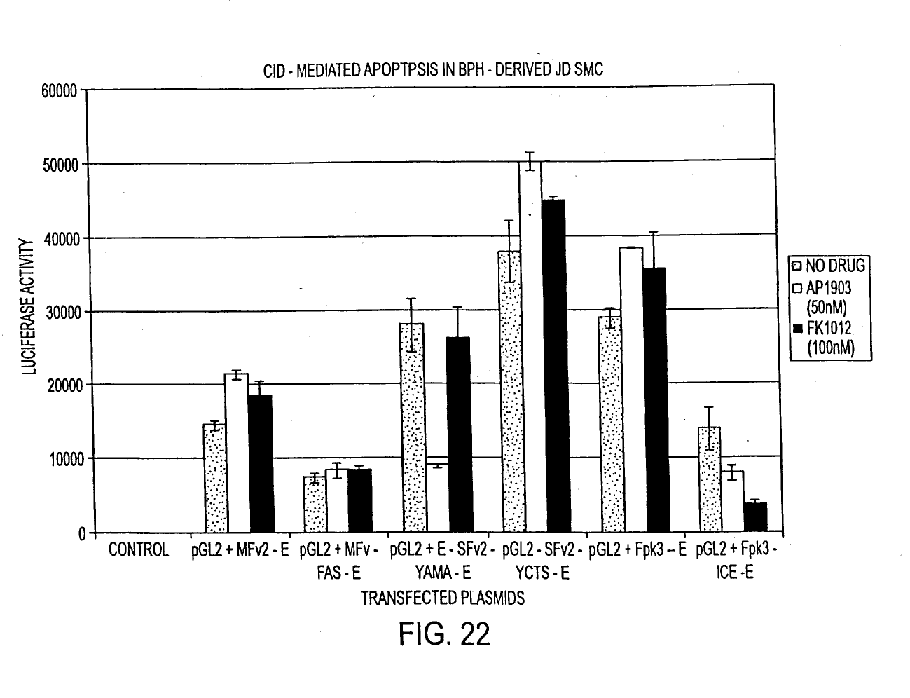

Conditional Fas, YAMA and a YAMA construct containing a single, inactivating point mutation were cotransfected with a luciferase reporter plasmid (pGL2) into JD and CR-2a cell lines. These are smooth muscle cell lines derived from prostate tissue samples taken from patients with BPH. Cells were maintained in control media±100 μM AP1903 for 24 hours. Cells were then lysed and assayed for luciferase activity as a marker of cell survival. The results obtained with CR-2a cells are presented in FIG. 21 and the results with JD cells are presented in FIG. 22.

There was no significant reduction in the percentage of surviving cells with or without AP1903 in cells transfected with conditional Fas or inactivated YAMA. In contrast, luciferase activity in cells transfected with conditional YAMA and treated with AP1903 fell from 668±67 to 119±13 representing an 82% reduction in reporter activity. This demonstrates the efficacy of the present invention in cell types found in BPH. It is worth noting that a conditional Fas-based treatment for BPH is not possible since the Fas construct was not effective in causing apoptosis in BPH derived cell lines. It is quite likely that Fas-based methods will be ineffective in many other cell types, thus severely limiting the utility of this approach.

Since different tissues or tumor lines are likely to be inhibited by one or more anti-apoptosis “gatekeeper” proteins, such as c-FLIP, Bcl-x_{L}, or IAPs, a broad repertoire of conditional pro-apoptotic proteins is likely to be useful to find the ADS(s) that work best for every cell type. By using the high-specificity CID, AP1903, and its cognate binding domain, Fv, we have developed a panel of highly sensitive ADSs based on Fas and the Fas signaling intermediates FADD, caspase-1, 3, and 8. Further, we have investigated several parameters relevant to caspase activation, (i) extent of crosslinking, (ii) interdomain flexibility, and (iii) intracellular localization. These improved ADSs now meet many, if not all, of the desiderata for a broadly applicable suicide switch: (a) They trigger apoptosis within hours of activation, (b) function independently of the cell cycle, (c) have low basal activity when expressed at minimal functional levels, (d) are based on endogenous proteins, and (e) can trigger apoptosis in multiple tissues independently if different CID/CBD combinations are used (Spencer et al., 1993; Belshaw et al., 1996). This data demonstrates that virtually all Fas signaling intermediates can be regulated by CIDs. The exquisitely sensitive AP1903-responsive Fas signaling intermediates described here should be able to function as suicide switches not only for gene therapy vectors but also for a variety of animal models based on temporally regulated and tissue-specific cell ablation.

### EXAMPLE 9

General experimental methods for Examples 10-15.

Plasmid construction.

All constructs were assembled from PfuI amplified fragments typically flanked by a 5′ XhoI and 3′ SalI site. PCR products were initially subcloned into pCR(r)-Blunt (Invitrogen) and sequenced. All expression plasmids were prepared by two-spin CsCl centrifugation and checked for expression by western blot. Parent expression plasmid pSH1/S-Fₚₖ3-E, containing 3 tandem copies of ˜330 bp hFKBP12_{P89,K90} and a 3′ hemaglutinin epitope (E) cloned into expression vector pSH1 (a high copy version of pBJ5), was described previously (MacCorkle et al., 1998). Inserts S-Fᵥ1-E, S-Fᵥ2-E, and S-Fᵥ3-E were made by substituting Fₚₖ3 with one to three tandem Fᵥs hFKBP12_{V36} previously described (Clackson et al., 1998; MacCorkle et al., 1998). In S-Fᵥᵢₛ1-E and S-Fᵥᵢₛ2-E, Fₚₖ3 is replaced with one or two copies of “short” Tinkered Fᵥ (“Fᵥᵢₛ”). Fᵥᵢₛ was amplified from Fᵥ using primers hFK5X: 5′-gcgacactcgag ggagtgcaggtgaaacc-3′ and hFKL3S1: 5′-acagtcgac tccggatccaccgccagattccagttttagaagctccac-3′. In S-Fᵥ1-Fᵥᵢₛ-E, Fᵥᵢₛ is subcloned into the 3′ SalI site of S-Fᵥ1-E. To make N2-Fᵥ2-E (and other variations), oligonucleotides 5′-tcgac cctaagaagaagagaaaggta c-3′ and 5′-tcgag tacctttctctcttcttagg g-3′, containing the nuclear localization sequence, PKKKRKV, from SV40 large T antigen (Boulikas 1993), were annealed and subcloned in tandem into the 5′ XhoI site of S-Fᵥ2-E. To make Mas70₃₄-Fᵥ2-E, the mitochondria-targeting sequence from pMas70 (residues 1-34) (Hase et al., 1984) was PCR amplified from M-Raf (Wang et al., 1996) using primers 5SCMAS70: 5′-cgacaccgcggccacc aμgaagagcttcattacaaggaac-3′ and 3XMAS70P: 5′-acactcgag ttgttgcaattggttgtaataatagtaggcaccgatggc-3′. The resulting ˜120 bp SacII/XhoI fragment (Mas70₃₄) was subcloned into SacII/XhoI-digested S-Fᵥ2-E. M-Fᵥ2-E and M-Fᵥ2-FAS-E were described previously (MacCorkle et al., 1998).

Human caspase 1 (Casp1), 3 (Casp3), and 8 (Casp8) inserts were PCR amplified from plasmids pcDNA3/hICE-AUI, pcDNA3/YAMA, and pcDNA3/FLICE, respectively, using primers, hICE5X: 5′-ccgacactcgag gccgacaaggtcctgaaggag-3′ and hICE3ST: 5′-agagtcgac ttaatgtcctggaagaggtagaaac-3′; YAMA5X: 5′-ccgacactcgag gagaacactgaaaactcagg-3′ and YAMA3S: 5′-cgtagagtcgac gtgataaaaatagagttcttttgt-3′; and FLICE5S; 5′-agagtcgac atggacttcagcagaaatctttatg-3′ and FLICE3S: 5′-cgtagagtcgac atcagaagggaagacaagtttttttc-3′. Resulting XhoI/SalI (caspase 1,3) or SalI (caspase 8) fragments were subcloned into the SalI sites of the appropriate vectors. Human FADD₁₂₅ and FADD₁₀₀ were PCR amplified from plasmid, pcDNA3/AU1-FADD, using 5′ primer FADD5X: 5′-ccgacactcgag gacccgttcctggtcgctgc-3′ and 3′ primer FADΔDD3X: 5′-ccgacactcgagcttggtgtctgagactttgagc-3′ or FADΔC3X: 5′-acactcgag tgctgcacacaggtcttcttccc-3′, respectively. FADD₈₀ is from the ˜240 bp XhoI/SalI fragment of FADD₁₂₅. Human ₂₅FADD₁₂₅ was amplified using primers Δ25Fad5x: 5′-acactcgag ctagcctctggcgcgtgggc-3′ and FADD5X. To make S-Fₚₖ3-FADD₁₂₅V82, residues 81 to 125 of FADD₁₂₅ were reamplified using primers 5SFADV82: 5′-cgcgtcgac gacgtcgaggcgggggcggcgg-3′ and FADΔDD3X. The resulting ˜140 bp SalI/XhoI fragment was then subcloned into the SalI site of pSH1/S-Fₚₖ3-FADD₈₀-E. Reporter plasmid SRα-SEAP was described previously (MacCorkle et al., 1998). Cloning sites are underlined and codons are separated.

Tissue Culture.

Jurkat-TAg cells were grown in RPMI 1640 medium, 10% Fetal Bovine Serum (FBS), 10 mM Hepes (pH 7.4), 100 units/ml penicillin, and 100 (g/ml streptomycin. HeLa cells were grown in Dulbecco's Modified Eagle Medium, 10% FBS and penicillin/streptomycin.

SEAP Assays.

Jurkat TAg cells (10⁷) in log phase growth were electroporated (950 μF, 250 V) with expression plasmid and 2 μg SRα-SEAP. After 24 hours, transformed cells were stimulated with CID. After an additional 20 hours, supernatants were assayed for SEAP activity as described previously (Spencer et al., 1993). Units of SEAP activity are reported directly and as a percentage of activity relative to no stimulation (“% Relative SEAP activity”). All experiments were repeated at least three times and representative experiments performed with duplicate samples are shown.

Western Blot Analysis.

Approximately 106 Jurkat TAg cells were lysed in 20 μl RIPA buffer (0.01MM TrisHCl pH 8.0, 140 mM NaCl, 1% Triton X-100, 1% sodium deoxychoIate, 0.1% SDS, 1:100 Protease Inhibitor Cocktail (Sigma P2714)) on ice for 20 minutes. Cell debris were pelleted and supernatants were boiled in 1:1 sample buffer (5% beta-mercaptoethanol in Bio-Rad Laemmli buffer) for 3-5 minutes. Alternatively, cells were lysed directly in 2× Laemmli buffer to detect nuclear proteins. Equal volumes of extracts were separated on a 15% SDS-PAGE gel. Membranes were blotted with anti-HA antibody, HA.11 (BABCO) and then with polygonal HRP-conjugated goat anti-mouse antibody. Bands were detected with SuperSignal® chemiluminescent substrate (Pierce).

Immunofluorescent Staining Protocol.

HeLa cells were plated at 2×10⁵ cells per 10-cm dish the night before transfection. Plated cells were incubated with 2 μg of indicated expression plasmids, containing various HA-tagged fusion proteins, resuspended in 3 μl FuGENE™6 (Boehringer-Mannheim) in OPTI-MEM® I media (Gibco-BRL). On day two, transfected cells were transferred to staining slides @10⁴ cells per spot and incubated overnight @37° C. Adhered cells were fixed in 4% paraformaldehyde (10′), permeabilized in minus 20° C. methanol (2′), rinsed 3× in PBS, and incubated for 1 hr @RT with HA.11 diluted 100× in PBS/3% serum. Following 3 PBS rinses, cells were incubated with FITC-conjugated goat anti-mouse polygonal Ig (Pharmingen) in PBS/3% serum for 45′ in the dark at RT. Following 3×10′ PBS rinsing, cells were treated with Vecta-shield anti-bleach mounting medium (Vector Laboratories, Burlingame, Calif.) and stored in darkness at 4° C. until analysis using a Multiprobe 2001 confocal system using Image Space software (Molecular Dynamics).

### EXAMPLE 10

Dimerization is sufficient for caspase 3 activation.

Following administration of CIDs, caspases containing amino terminal CBDs should be crosslinked, leading to intermolecular processing in some cases (FIG. 23A). Since pro-domains with attached CBDs are removed, CID-activated proteins should be indistinguishable from physiologically activated caspases.

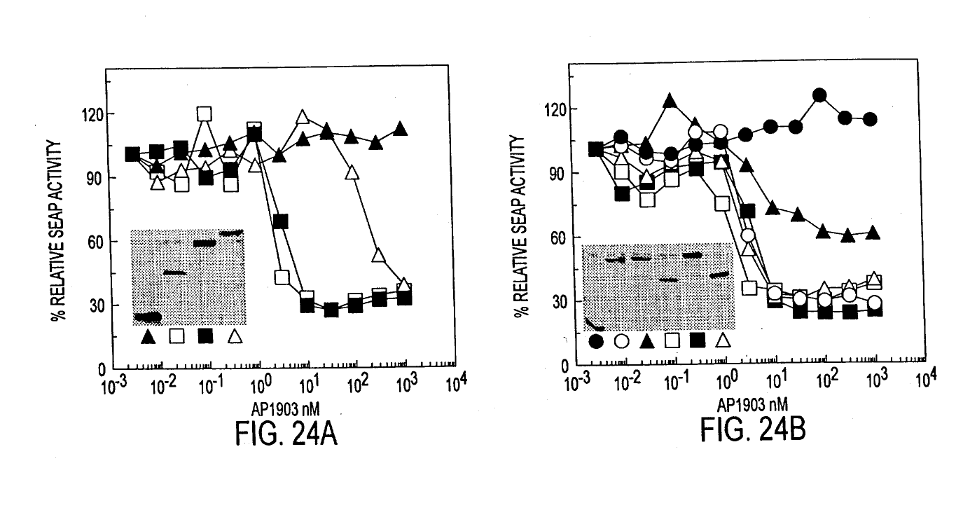

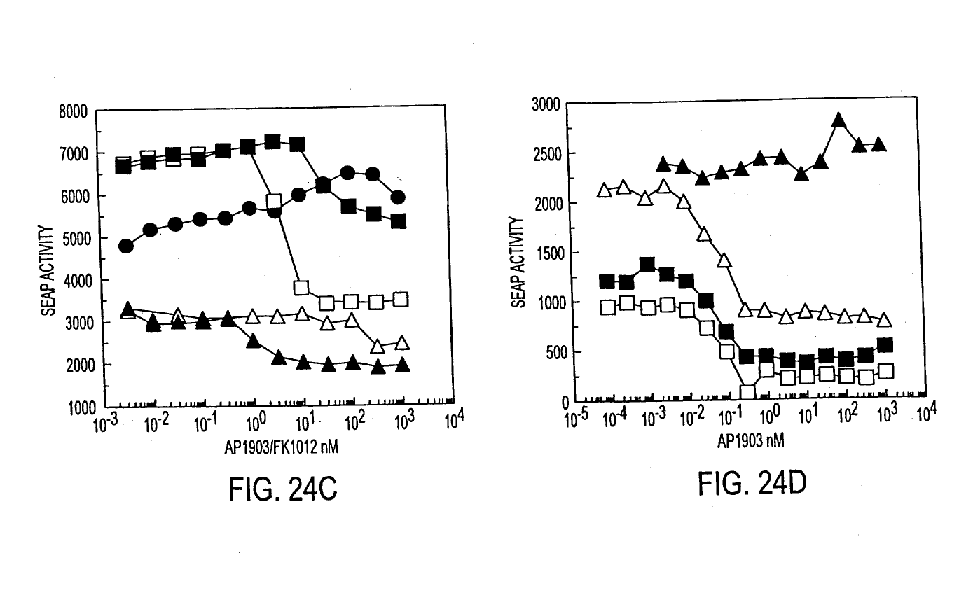

To determine the number of FKBPs that are needed for optimal CID-mediated caspase activation, we attached 0, 1, 2, or 3 copies of FKBP12V36 (abbreviated “Fᵥ”) to the amino terminus of caspase-3 (FIG. 23B). Since AP1903 binds with high affinity to Fv (K_{d}˜0.1 nM) (Clackson et al., 1998) but with low affinity to wild-type FKBP12 (K_{d}=67 nM), high specificity for Fᵥ is achieved. Individual constructs were transiently transfected into Jurkat TAg cells along with the reporter plasmid, SRα-SEAP, containing secreted alkaline phosphatase (SEAP) under the transcriptional control of the constitutively active promoter, SRα. Twenty-four hours later, cell aliquots were treated with increasing amounts of AP1903. After an additional twenty hours, cell supernatants were assayed for reporter activity. Although all constructs were expressed at comparable levels (FIG. 24A), constructs containing either one or two Fᵥs were equally sensitive to AP1903 as reflected by the dramatic decrease in SEAP activity (IC₅₀˜3 nM), whereas constructs containing three Fᵥs were much less sensitive (IC₅₀˜150 nM) to AP1903. S-Fᵥ3-Casp3 is however still sensitive to the larger (i.e. MW˜1800 D) CID, FK1012 (IC₅₀˜20 nM; data not shown). Reductions in reporter activity by this assay faithfully reflect apoptosis as determined by flow cytometry ((MacCorkle et al., 1998) and data not shown). These results demonstrate that dimerization of caspase-3 is sufficient for its activation while excess crosslinking by the relatively small CID, AP1903 (MW˜1200 D), may “lock” caspases into inactivatable conformations.

### EXAMPLE 11

Caspase-3 activation is not sterically hindered by amino terminal FKBP12.

Two possible, non-exclusive, models can account for CID-mediated caspase activation: (i) CIDs increase the proximity of procaspases, increasing the likelihood that transproteolysis will occur and (ii) CIDs actively maintain the correct orientation for caspase processing. If model (i) is correct, then molecules should be relatively insensitive to orientation and spacing between FKBPs and caspases. If model (ii) is correct, then the converse is true. To test these two possibilities, we engineered a small, six amino acid G-S-G-G-G-S linker/spacer between Fᵥ and caspase-3 permitting increased flexibility (FIG. 23B). Again, constructs were transiently transfected into Jurkat TAg cells and treated after 24 hours with AP1903. We observed no significant difference in the dose response to AP1903 between constructs with or without the small linker, regardless of whether one or two Fᵥs were fused to caspase-3 (compare S-Fᵥ1-Casp3 with S-Fᵥᵢₛ1-Casp3 and S-Fᵥ2-Casp3 with S-Fᵥ1-Fᵥᵢₛ1-Casp3; FIG. 24B). In contrast, construct S-Fᵥᵢₛ2-Casp3, containing 2 linkered FKBPs (“Fᵥᵢₛ”) was less sensitive to AP1903, presumably because the increased rotational freedom of this construct reduces the probability that the correct conformation for cleavage occurs. These results support the model that the orientation by which caspases are brought together is important for their activation, and that AP1903 fortuitously crosslinks S-Fᵥ1-Casp3 in an appropriate orientation that is not improved, and may be decreased, by increasing the flexibility and rotational freedom of the crosslinked molecules.

### EXAMPLE 12

The activation of caspase-1 and -8 by AP1903 is sterically hindered by amino terminal FKBP12.

While Fᵥ2-Casp3 does not require a flexible linker for efficient activation by AP1903, caspases-1 and -8 fused to two Fvs, S-Fᵥ2-Casp1 and S-Fᵥ2-Casp8, cannot be activated efficiently by AP1903 (IC₅₀ caspase 1˜200 nM; FIG. 24C and data not shown). However, the larger CID, FK1012, can activate S-Fᵥ2-Casp1 (IC₅₀1 nM), despite the lower affinity (by ˜ten-fold) of FK1012 for Fᵥ versus AP1903 and that FK1012 does not discriminate against endogenous FKBPs. In contrast, the advantages of AP1903 versus FK1012 are readily apparent on the activation of S-Fᵥ2-Casp3. These results imply that either AP1903 brings S-Fᵥ2-Casp1 and Fᵥ2-Casp8 into unfavorable orientations for processing or that steric hindrance prevents the efficient crosslinking of these FKBP/caspase chimeras.

Therefore, to increase AP1903 sensitivity, we fused caspase-1 and 8 to the linkered FKBP, Fᵥᵢₛ, as above. As hypothesized, the use of a flexible linker in S-Fv1-Fᵥᵢₛ1-Casp1 (FIG. 24D) and in S-Fv1-Fᵥᵢₛ1-Casp8 (FIG. 24E) led to alleles of caspase-1 and -8 that were exquisitely sensitive to AP1903 (IC₅₀ caspase-1≦100 pM; IC₅₀ caspase-8˜100 pM). Again, providing too much flexibility, as in S-Fᵥᵢₛ2-Casp1, reduced the responsiveness to AP1903 (FIG. 24F), and adding the longer linker, G-G-S-G-G-G-S-G-G-G, almost completely abrogated AP1903 responsiveness (data not shown). In each case, however, the basal drug-independent cytotoxicities of linkered caspase-1 and -8 constructs were the same as unlinkered constructs, implying that amino terminal Fvs do not sterically hinder stochastic caspase interactions. Nevertheless, since S-Fᵥ1-Fᵥᵢₛ1-Casp1 and -Casp8 are highly sensitive to AP1903, reducing protein expression to levels that are still sensitive to AP1903 (FIGS. 24D and 24E) can largely eliminate these basal toxicities.

### EXAMPLE 13

Crosslinking the death effector domain of FADD is sufficient for triggering apoptosis with reduced basal toxicity.

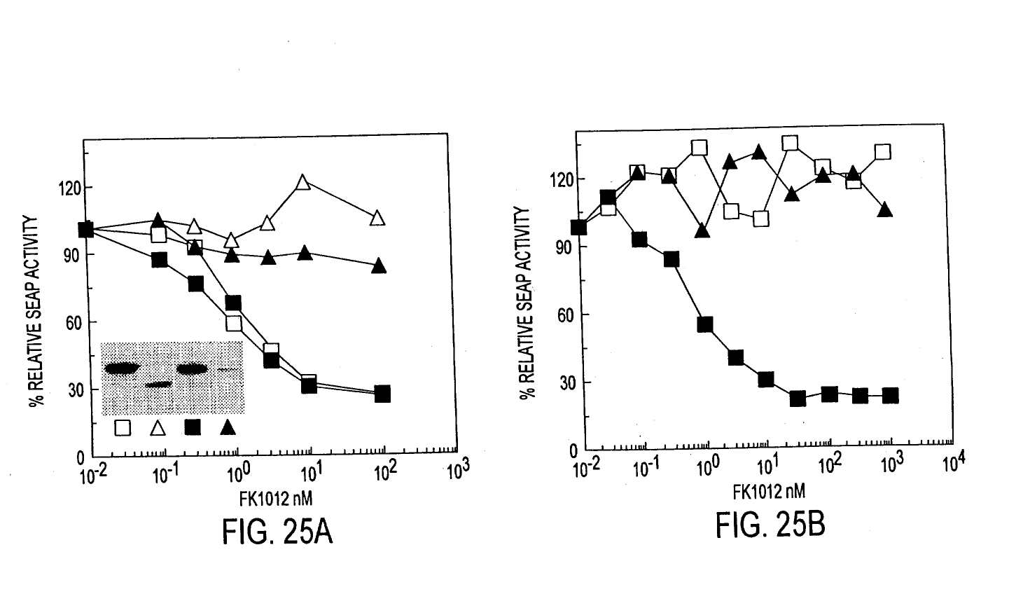

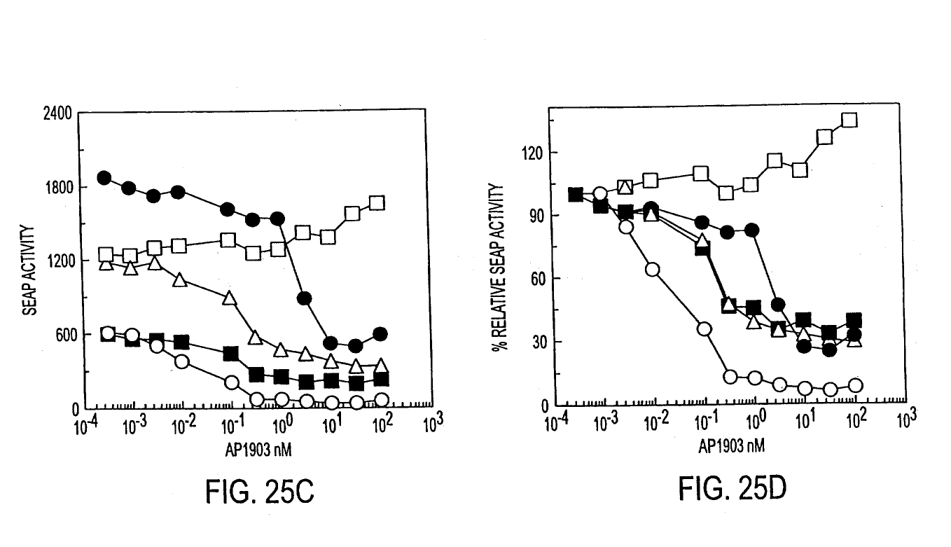

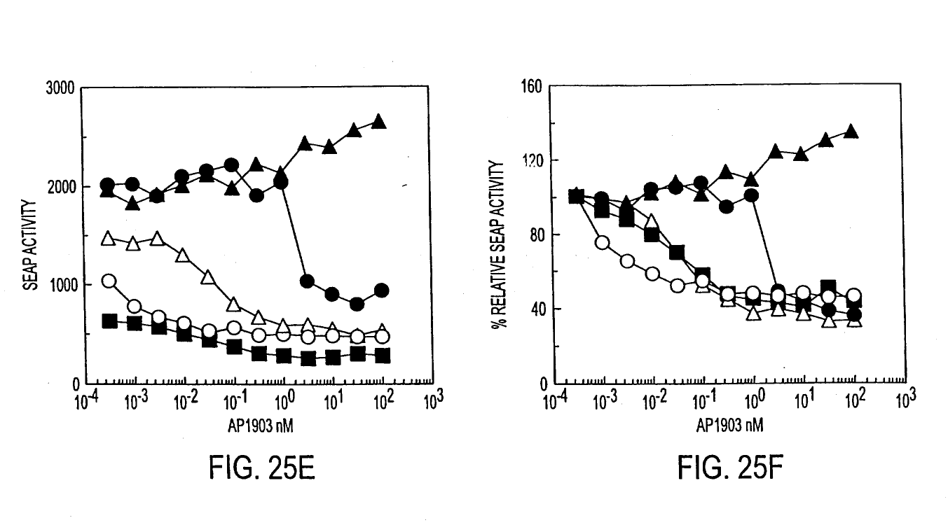

Since caspases, such as caspase-1 and 8, can have high basal activity when overexpressed and Fas is autotoxic due to the tendency of death domains to self-associate (Boldin et al., 1995), we investigated whether CID-mediated crosslinking of the adapter molecule FADD could trigger apoptosis with lower basal activity. Therefore, we fused the amino terminus of FADD (FADD125; residues 1-125), containing the DED, to a trimer of FKBP12_{P89,K90} to get S-Fₚₖ3-FADD₁₂₅. As above, we cotransfected reporter plasmid into Jurkat TAg cells along with S-Fₚₖ3-FADD₁₂₅ or variants, including S-Fₚₖ3-Δ25FADD₁₂₅ (residues 26-125), S-Fₚₖ3-FADD₁₀₀ (residues 1-100), or S-Fₚₖ3-FADD₈₀ (residues 1-80). While crosslinking FADD125 and FADD₁₀₀ led to FK1012-dependent diminution of reporter activity, further truncation of FADD, as in Δ25FADD₁₂₅ and FADD₈₀, eliminated FK1012-dependent toxicity (FIG. 25A). The lower stability of these truncated proteins may contribute only partially to this lack of activity (FIG. 25A, inset) since S-Fₚₖ3-FADD₁₂₅ and -FADD₁₀₀ still function better than S-Fₚₖ3-FADD₈₀ even after normalizing transfections for steady-state protein levels (data not shown). Control point mutant, S-Fₚₖ3-FADD₁₂₅V82, was also unable to trigger apoptosis following dimerization (FIG. 25B). Thus, crosslinking the DED of FADD is sufficient to trigger the Fas pathway.

To quantitate the differences in specific and basal activities between AP1903-inducible versions of caspases 1, 3 and 8, FADD, and Fas, equivalent amounts of each expression vector were transfected into Jurkat cells. Twenty hours after AP1903 administration, SEAP activity was determined as above (FIG. 25C) and results were further normalized to drug-free control wells (FIG. 25D). Consistent with all previous experiments, CID independent basal toxicities had the following ranking: Fas, caspase-8, caspase-1>FADD₁₂₅>caspase-3. (The low, but reproducible, level of autotoxicity due to overexpression of non-chimeric FKBP12 (e.g. S-Fᵥ1-Fᵥᵢₛ1) is relieved by adjacent protein domains or by CIDs and probably reflects interaction with a subset of cytoplasmic proteins.) Sensitivity to AP1903 follows a somewhat different order than basal toxicity: caspase-1 (IC₅₀˜50 pM)>Fas, FADD, caspase-8 (IC₅₀˜200 pM)>caspase-3 (IC₅₀˜2 nM). Thus, caspase-1 is likely to be the most effective ADS for most applications due to its exquisite sensitivity, while caspase-3 may be more appropriate when long-term expression is required due to low basal activity.

### EXAMPLE 14

Plasma membrane targeting of caspase-3 increases its CID sensitivity and basal activity.

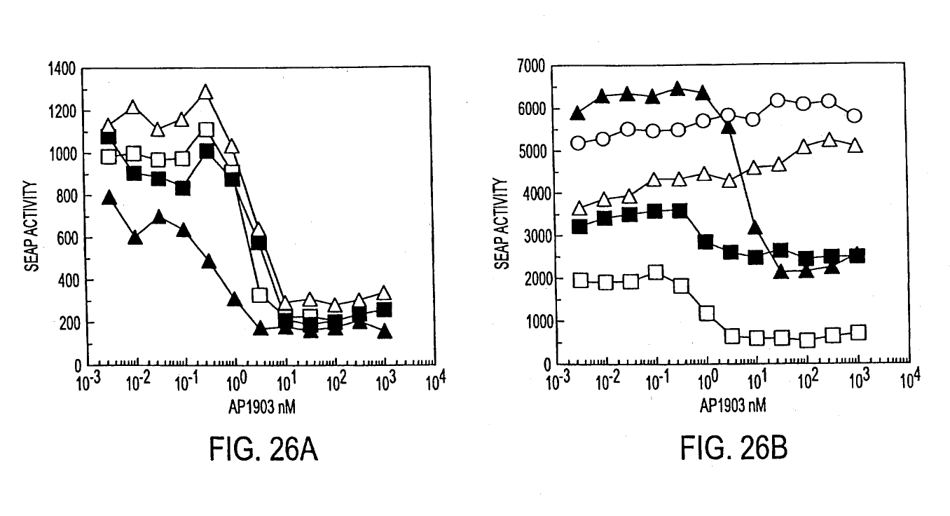

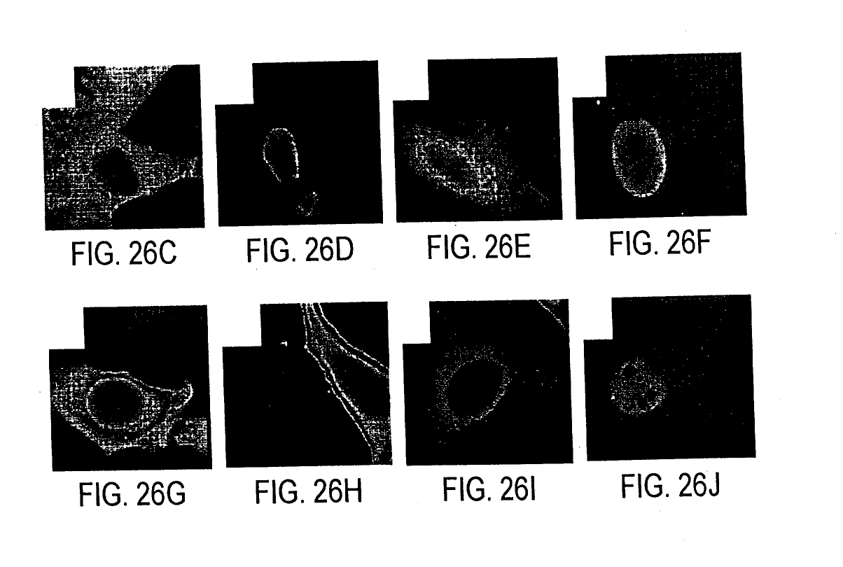

Since FKBP/caspase-3 chimeras display very low basal activity, we considered the possibility that intracellular localization of caspase-3 might increase CID sensitivity without a commensurate increase in basal toxicity. Therefore, Fᵥ2-Casp3 was fused to a myristoylation-targeting sequence (M) as in M-Fᵥ2-Casp3, a mitochondrial-targeting sequence as in Mas70₃₄-Fᵥ2-Casp3, or a nuclear-localization sequence as in N2-Fᵥ2-Casp3. Once again, the various constructs were transfected into Jurkat cells and assayed for inducible apoptosis. Surprisingly, only the plasma membrane-localized chimeric caspase-3 was significantly more sensitive to AP1903 than the non-localized construct, S-Fᵥ2-Casp3 (IC₅₀˜300 pM vs. ˜3 nM; FIG. 26A). Moreover, M-Fᵥ2-Casp3 was significantly more autotoxic than the other Casp3 constructs even when four-fold less plasmid was transfected (FIG. 26A). While reducing the expression levels of M-Fᵥ2-Casp3 reduces basal activity, AP1903 sensitivity disappears before basal activity does, rendering low, completely non-toxic levels insufficient for triggering apoptosis (FIG. 26B). Further, we observed that plasma membrane-localized FADD and caspase-8 were even more autotoxic than membrane-localized caspase-3, consistent with our observations that soluble forms of these proteins have higher basal activity than caspase-3 chimeras.

To ensure that the (M, Mas70₃₄, and N2) targeting sequences used in this study conferred predicted localization to heterologous proteins, immunofluorescence was performed on cells transiently transfected with the differentially targeted, HA-tagged caspase-3 alleles or with epitope (E)-tagged control proteins. Since intracytoplasmic staining in Jurkat cells is difficult to visualize due to a low cytoplasm:nucleus ratio, caspase-3-sensitive HeLa cells were used (MacCorkle et al., 1998). As expected, nontargeted S-Fᵥ2-Casp3-E is distributed throughout the cytoplasm (FIG. 26C); however, staining is somewhat punctate, suggesting possible intracellular membrane interactions. Plasma membrane targeted M-Fᵥ2-Casp3-E stains at the plasma membrane; however, transfected cells are primarily shrunken and apoptotic, reflecting the high basal toxicity of this construct (FIG. 26D). Mitochondria-targeted Mas70₃₄-Fᵥ2-Casp3-E stains in a perinuclear punctate pattern consistent with mitochondria staining (FIG. 26E), and nuclear-targeted N2-Fᵥ2-Casp3-E stains in the nucleus (FIG. 26F). Control constructs, S-Fᵥ2-E (FIG. 26G), M-Fᵥ2-E (FIG. 26H), Bcl-x_{L}-E (FIG. 26I), and Gal4-VP16-E (FIG. 26J), all localized to their predicted intracellular locations.

### EXAMPLE 15

Nuclear-targeted caspases trigger apoptosis.

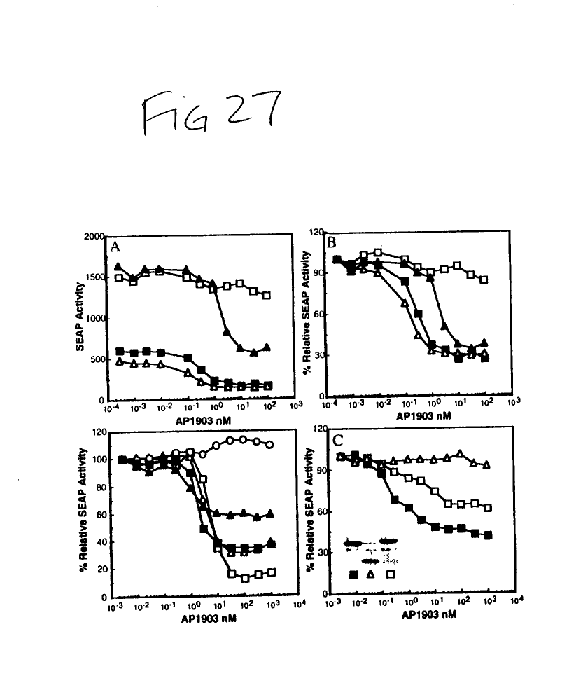

Since multiple caspase targets are localized in the nucleus, such as poly(ADP-ribose) polymerase (PARP), lamin A and B. DNA-dependent protein kinase catalytic subunit (DNA-PK_{ca}) histone H1, MDM2, and topoisomerases, it is not surprising that nuclear activation of caspase-3 can trigger apoptosis. In order to test whether caspase-3 is unique in this ability, we also targeted caspase-1 and 8 to the nucleus and triggered their activation with AP1903. Interestingly, all three caspases were fully functional in the nucleus and had basal activities and AP1903 sensitivities similar to their cytoplasmic activities (FIG. 27A,B). Although immunofluorescence studies suggested that nuclear targeting is efficient, we cannot rule out that a small amount of cytoplasmic protein is responsible for this cytotoxicity. To minimize this possibility we titrated both cytoplasmic and nuclear Fᵥ2-Casp3 and compared their AP1903 sensitivities. Consistent with localization studies and FIG. 26A, N2-Fᵥ2-Casp3 can trigger apoptosis with a similar AP1903 dose response relative to S-Fᵥ2-Casp3, even at low levels (FIG. 27C). Nuclear N2-Fᵥ-Fᵥᵢₛ-FADD₁₂₅, however, was unable to activate apoptosis as efficiently as cytoplasmic S-Fᵥ-Fᵥᵢₛ-FADD₁₂₅, likely reflecting the fact that its normal interaction with cytoplasmic caspase-8 does not normally occur in the nucleus (FIG. 27D). Thus, cleavage of nuclear substrates by caspases in intact cells is sufficient to trigger apoptosis.

We find that dimerization of caspase-3 is sufficient for maximum CID sensitivity, while higher order multimerization is somewhat more efficient for caspase-1 activation. We also find that optimized activation of caspase-1, -8, and FADD₁₂₅ by AP1903 (but not the larger CID, FK1012) requires a short G-S-G-G-G-S linker between the cognate CBD (i.e. Fᵥ) and the pro-caspase. The crystal structure of AP1903 bound to two Fᵥs reveals that the FKBP12_{V36} moieties are brought into closer proximity than the two FKBP12-moieties of FK1012A/FKBP12 (M. Gilman, personal communication). Thus, the more “intimate” AP1903-mediated Fᵥ interactions may “lock” crosslinked chimeric proteins into conformations that are incompatible with their activation. Interestingly, constructs with two interdomainal linkers (e.g. S-Fᵥᵢₛ2-Casp3) or with a longer G-G-S-G-G-G-S-G-G-G linker (data not shown) are less sensitive to activation, perhaps due to too much flexibility. This may imply that CIDs do more than increase the proximity of proteins; they could also hold proteins in the correct (or incorrect) orientation for activation. Further, we find that plasma membrane localization of conditional caspase-3, 8 or FADD₁₂₅ increases their sensitivity for AP1903 by about tenfold, while simultaneously increasing their basal activities. Interestingly, mitochondrial localization of Mas70₃₄-Fᵥ2-Casp3 did not increase its basal activity relative to cytoplasmic S-Fᵥ2-Casp3 even though some anti-apoptosis caspase-3 targets, like Bcl-2 and Bcl-x_{L} are mitochondria localized (Cryns et al., 1998). Perhaps the topography of the mitochondrial outer membrane, distinct membrane fluidity, or large surface area may reduce the local concentration of Mas70₃₄-Fᵥ2-Casp3 relative to M-Fᵥ2-Casp3.

### EXAMPLE 16

Construction of gene therapy vectors expressing CID-apoptosis factors.

Any method of delivering a nucleic acid encoding a chemically inducible apoptosis factor may be used to practice the present invention. These methods may involve the use of a gene therapy vector. A gene therapy vector is any molecule which, when delivered to a target cell, is capable of causing the expression of a desired molecule. In the instant invention, the desired molecule is the chemically inducible apoptosis factor. Preferred embodiments of gene therapy vectors include viruses, plasmids and fragments of nucleic acid. The chemically inducible apoptosis factor may be included in the vector to be used directly as the therapeutic gene or may be incorporated into the gene therapy vector as an “artificial death switch” or safety mechanism.

Those skilled in the art will readily appreciate that molecules other than the ADS of the present invention may be incorporated into the gene therapy vector in addition to the ADS. For example, a vector expressing the ADS and an immunostimulatory compound may be constructed. The immunostimulatory compound may be an interleukin, cytokine, colony stimulating factor or the like.

In a preferred embodiment, the gene therapy vector of the present invention will be a replication restricted virus. By replication restricted it is meant that the virus is not capable of producing infective progeny virus in the target cell. In a most preferred embodiment the replication restricted virus will be an adenovirus. In an alternative embodiment, the virus used as a gene therapy vector may be capable of producing infectious progeny; however, such progeny may be sufficiently attenuated so as to be unable to produce a symptomatic viral disease. Other viral vectors that may be used to practice the instant invention include, but are not limited to, vaccinia virus, herpes virus, retroviruses, adeno-associated virus and any other virus capable of entering the specific cell type desired to be treated and expressing the desired molecule.

The viral vectors of the instant invention may be administered by any route customarily used in gene therapy applications. Thus, they may be administered intramuscularly, parenterally, orally, subcutaneously or topically so long as they result in uptake of the vector into the desired cell type. The viral vectors of the present invention may be administered as aerosol inhalants when used to treat lung tissue or as entericly coated capsules when used to treat intestinal tissue.

Vectors of the present invention may include regulatory sequences to control the expression of the chemically induced apoptotic factor. These regulatory sequences may be eukaryotic or prokaryotic in nature. They may result in the constitutive expression of the apoptosis factor such that the factor is continuously expressed upon entry of the vector into the cell. In a preferred embodiment, the regulatory sequence will be a tissue specific promoter such that the expression of the ADS will be substantially greater in the target tissue type compared to other types of tissue. Alternatively, the regulatory sequences may be inducible sequences. Inducible regulatory sequences are well known to those skilled in the art and are those sequences that require the presence of an additional inducing factor to result in expression of the CID-apoptotic factor. Examples of suitable regulatory sequences include, but are not limited to, binding sites corresponding to CID-regulated tissue-specific transcription factors based on endogenous nuclear proteins, sequences that direct expression in a specific cell type, the lac operator, the tetracycline operator and the steroid hormone operator. Any inducible regulatory sequence known to those of skill in the art may be used in conjunction with the present invention.

Plasmid based vectors may also be used to administer the present invention. Plasmid vectors may be administered by any method known to those skilled in arts such as transfection, lypofection, cell fusion, or injection at high speed. Plasmid vectors may also contain regulatory sequences. In addition, they may contain other genes used to mark the presence of the plasmid in a cell or to select for the presence of the plasmid in a cell. Suitable marker and selection genes are known to those skilled in the art.

Nucleic acids may be directly used to administer the apoptosis factors of the present invention. The nucleic acid may be DNA or RNA. The nucleic acid may incorporate chemical groups that alter the physical characteristics of the nucleic acid. For example, the internucleotide phosphate ester may be optionally substituted with sulfur so as to retard the degradation of the nucleic acid molecule. The nucleic acid may be introduced into the target cell by any means known to those skilled in the art.

Although the present invention is particularly useful for in vivo applications it may also be used for ex vivo applications. In ex vivo applications, nucleic acids encoding the chemically induced apoptotic factor may be stably integrated into the genome of a cell. The cell can then be expanded to produce a population of cells containing the chemically induced apoptotic factor. Alternatively, the nucleic acids encoding the apoptosis factor may be maintained in the cell but not integrated into the genome. Those of skill in the art will appreciate that this process may require the addition of various other markers and selectable resistance genes in order to ensure that the entire population of expanded cells contains the chemically induced apoptotic factor. Various markers that may be used include hprt, neomycin resistance, hygromycin resistance and the like.

Once the vector has been administered and taken up by the appropriate cell type, the vector may cause the expression of the chemically induced apoptotic factor. Once expressed, the factor can be activated by the addition of the appropriate inducing ligand. The ligand may be administered in any fashion known to those skilled in the art including intramuscularly, orally, parenterally, subcutaneously or topically so long as the ligand is brought in contact with the cell containing the chemically inducible apoptotic factor.

### EXAMPLE 17

Construction of an adenovirus expressing a chemically inducible apoptosis factor.

The E-Fᵥ2 YAMA-E construct described previously was placed under the control of the CMV promoter and inserted into an adenoviral recombination vector. The adenoviral recombination vector contained a copy of the D1 gene from adenovirus. The CMV promoter -E-Fᵥ2-YAMA-E construct was inserted such that the Fᵥ2 construct was flanked on both sides by nucleotide sequences from the E1 gene.

The plasmid was transfected into adenovirus infected cells and a recombinant adenovirus expressing the chemically inducible apoptosis factor was isolated and purified by standard methods.

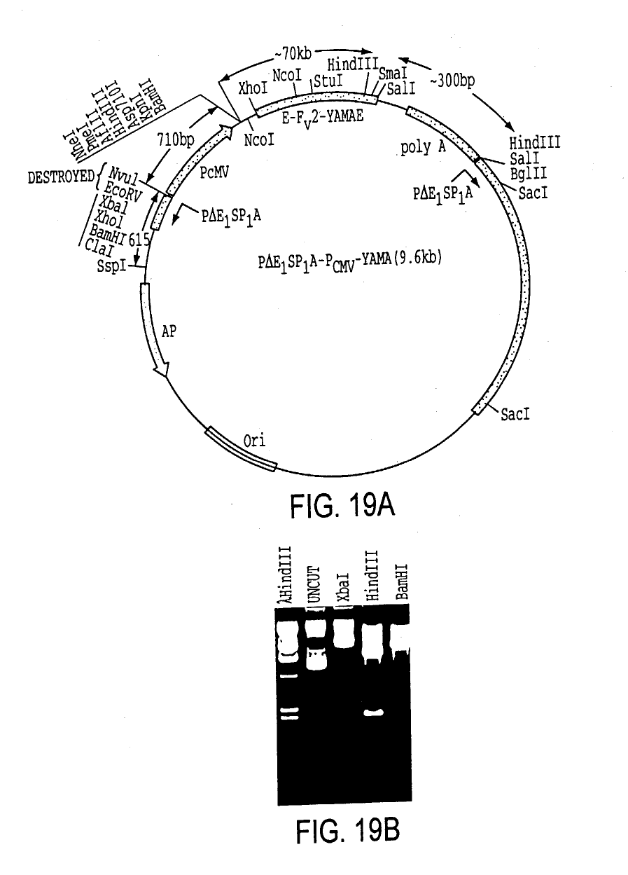

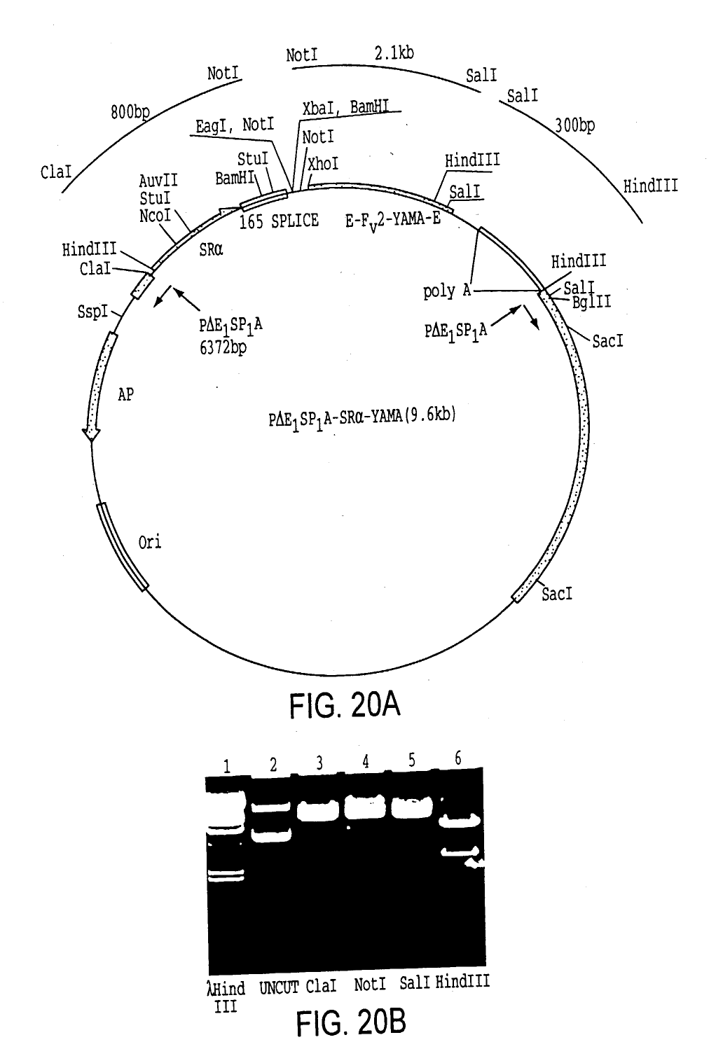

FIG. 19 A shows a plasmid map of the adenoviral recombination vector used to construct an adenovirus expressing E-Fᵥ2-YAMA-E under control of the CMV promoter and incorporating the 16S splice junction to improve the efficiency of mRNA processing (Takabe, et al. Mol. Cell. Bio. 8:466-472, 1988). Panel B slows the results of a restriction analysis of the plasmid. FIG. 20 A shows a plasmid map of the plasmid used to construct a recombinant adenovirus expressing E-Fᵥ2-YAMA-E under the control of the SRα promoter. Panel B shows the results of a restriction analysis of the plasmid.

### EXAMPLE 18

Construction of Additional Adenovirus Constructs Containing Conditional Caspase 1 or 3

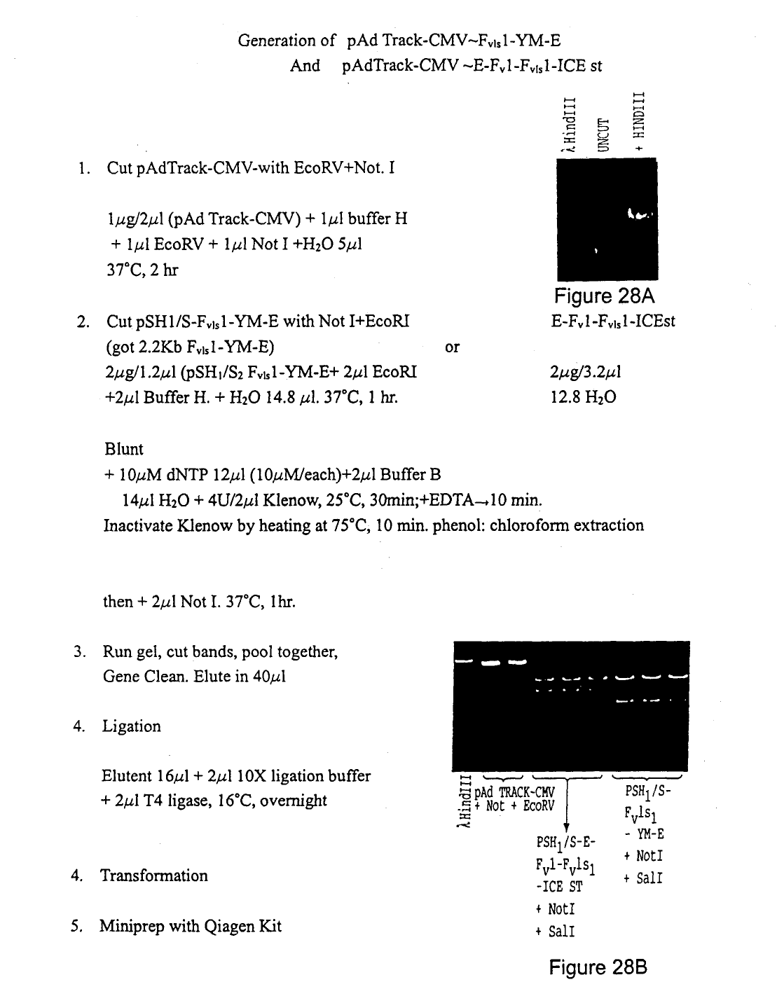

I. Construction of pAdTrack-CMV-Fᵥᵢₛ1-Yama-E (FIG. 28)

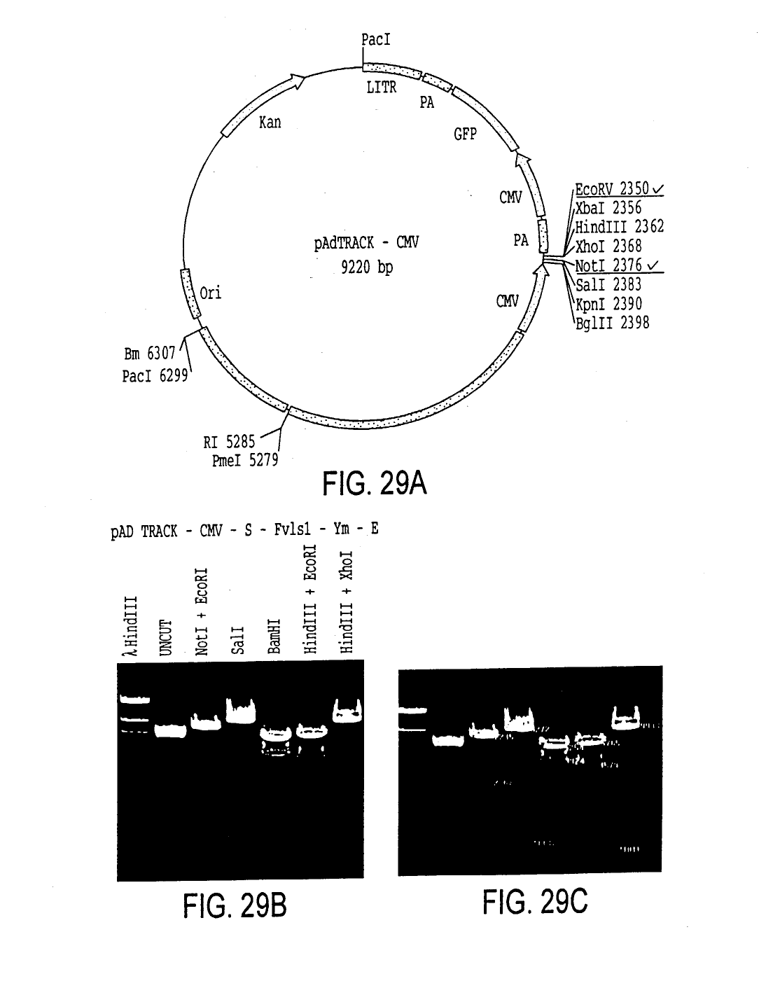

1. Digest pAdTrack-CMV with Eco RV and Not I. Purify the 9.2 kb vector by agarose electrophoresis and GeneClean (FIG. 29a-29c).

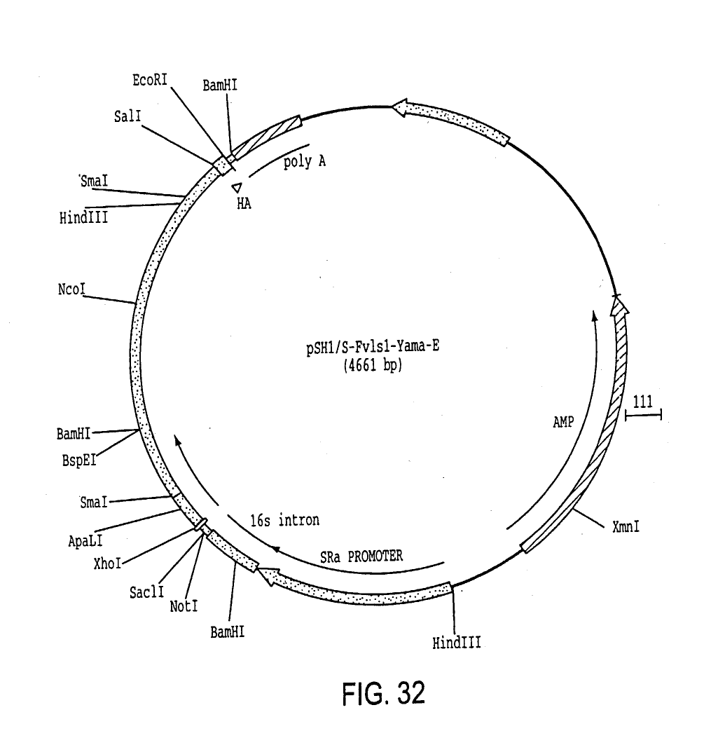

2. Digest pSH1/S-Fᵥᵢₛ1-Yama-E (FIG. 32) with Eco RI and Not I. Blunt the Eco RI end. Purify the 1.2 kb fragment by agarose electrophoresis and GeneClean.

3. Ligate the above two fragments and transform XL1-blue with the ligation. Chose several colonies, do Miniprep and check with Sal I, Not I+Eco RI, Hind III+Eco RI, Hind III+Xho I. See FIG. 29b.

II. Construction of pAdTrack-CMV-E-Fᵥ1-Fᵥᵢₛ1-ICEst (FIG. 28)

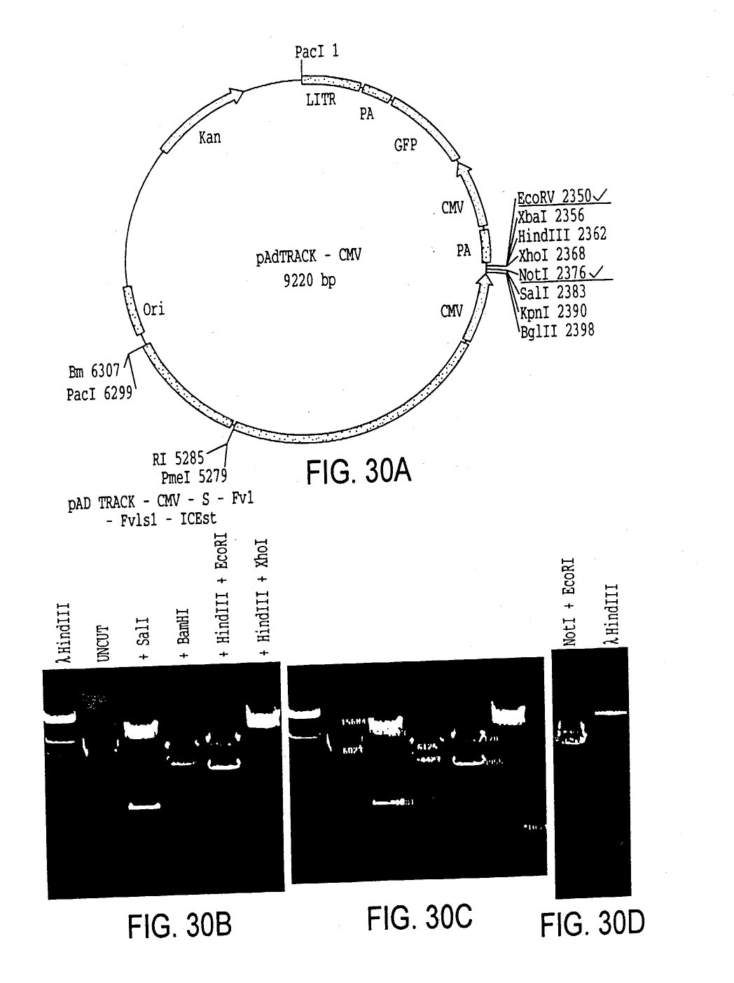

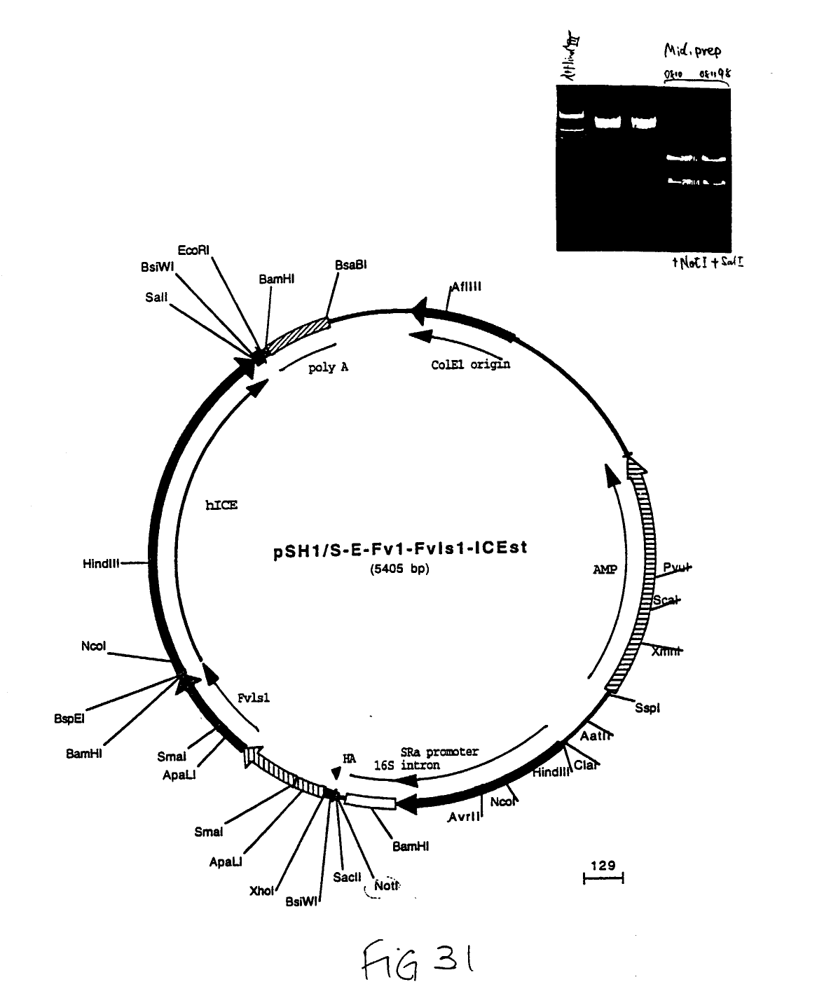

1. Digest pAdTrack-CMV with Eco RV and Not I. Purify the 9.2 kb vector by agarose electrophoresis and GeneClean (FIG. 30a-30c).

2. Digest pSH1/S-E-Fᵥ1-Fᵥᵢₛ1-ICEst (FIG. 31) with Eco RI and Not I. Blunt the Eco RI end. Purify the 2.2 kb fragment by agarose electrophoresis and GeneClean.

3. Ligate the above two fragments and transform XL1-blue with the ligation. Chose several colonies, do Miniprep and check with Sal I, Not I+Eco RI, Hind III+Eco RI, Hind III+Xho I. See FIG. 30b.

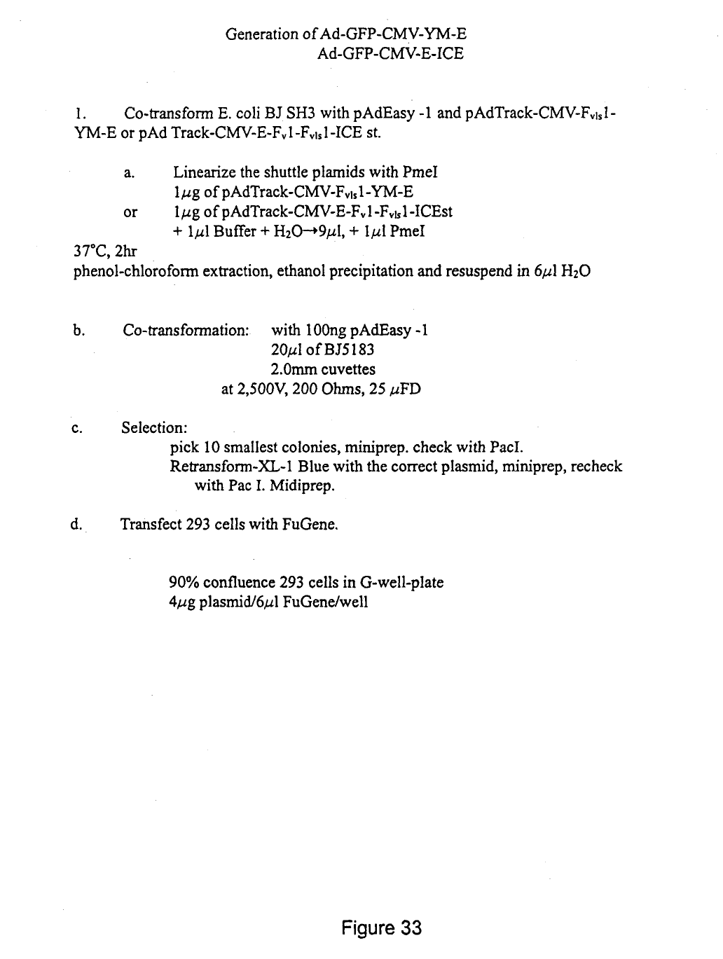

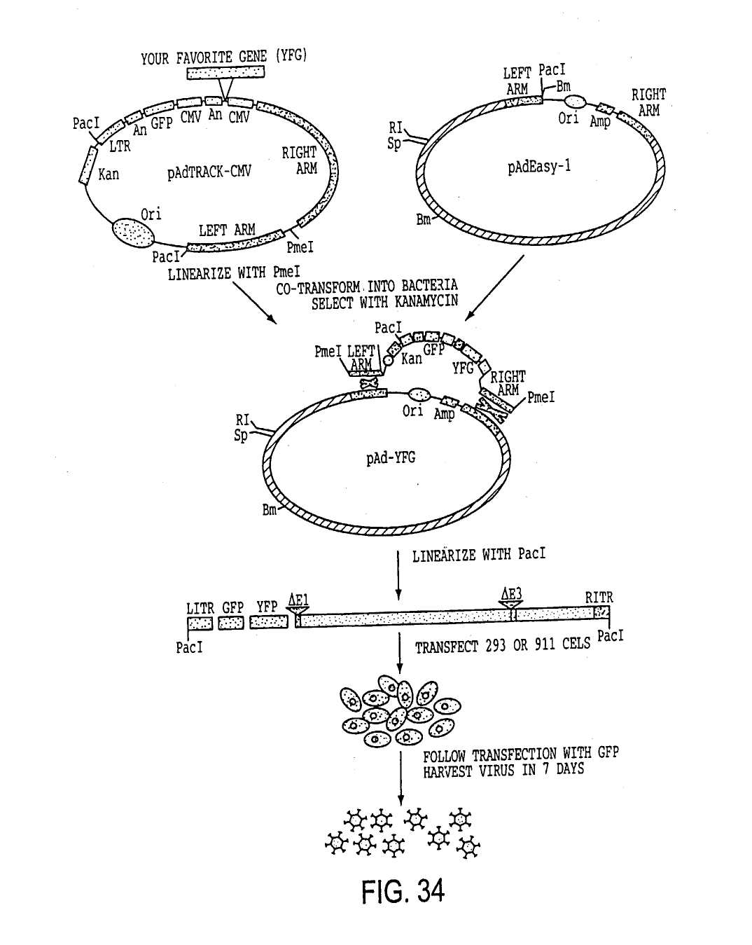

III. Generation of ADV-GFP-CMV-Yama-E (FIGS. 33 and 34)

1. Linearize 1 ug pAdTrack-CMV-Fᵥᵢₛ1-Yama-E with Pme I. Purify it by phenol-chloroform extraction, ethanol precipitation and resuspend in 6 μl H2O.

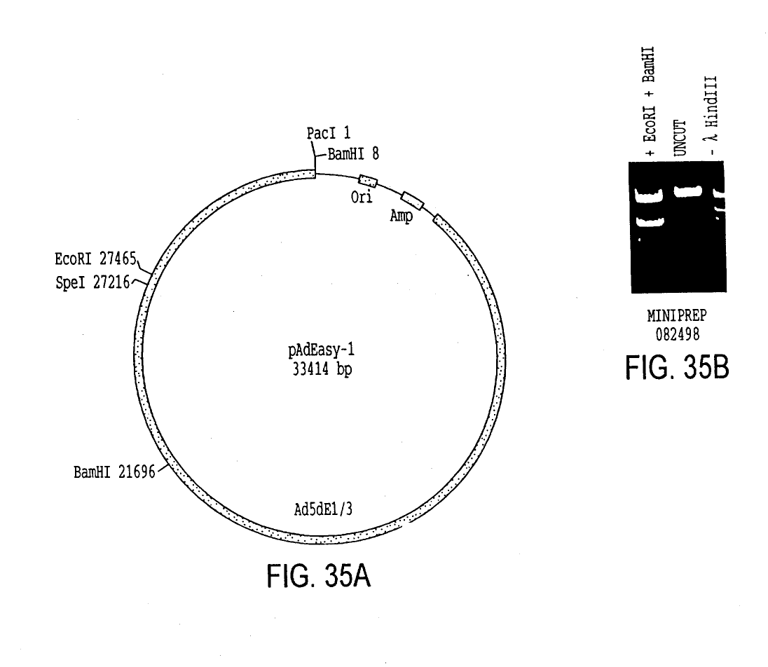

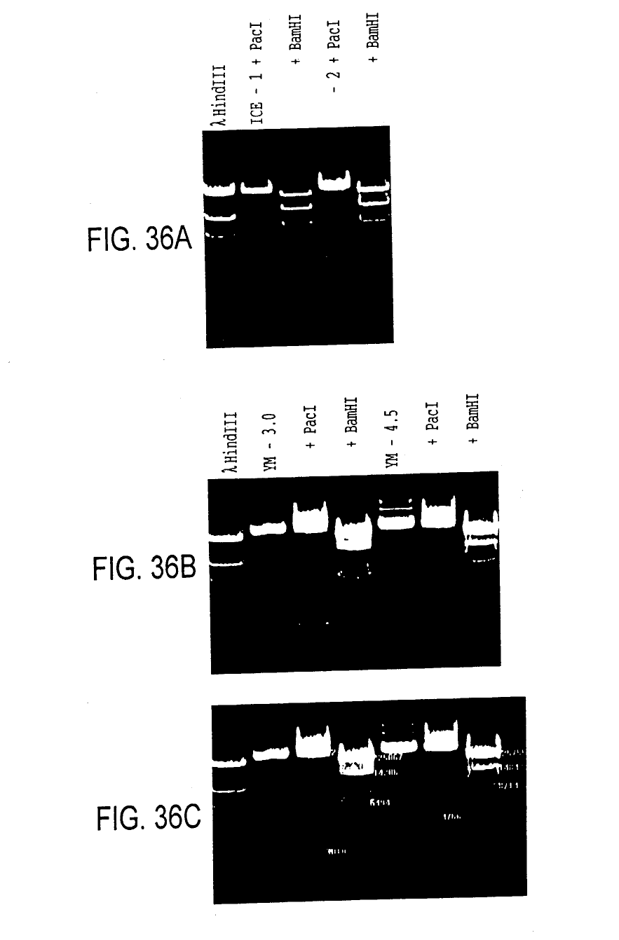

2. Mix it with 100 μg p AdEasy-1 (FIG. 35) (in 1 μl), Co-transform 20 μl E. coli BJ5183 competent cells with GenePulser at 2,500 V 200 Ohms, 25 uFD.

3. Pick up 20 smallest colonies. Do Miniprep and check with Pac I. Candidate clones usually yield a large fragment (near 30 kb), plus a smaller fragment of 3.0 kb or 4.5 kb.

4. Re-transform the correct recombinant plasmids into Xl1-blue. Midiprep with Qiagen kit.

5. Transfect 293 cell by the recombinant plasmid with FuGene. 4 μg DNA/6 μl FuGene/well (6 well-plate). Check GFP expression with fluorescent microscope.

6. Harvest the cells when 30% of them are detached; Spin down the cells; use the supernatant for next infection. Repeat infection for several rounds.

7. Collect the cells, repeat freeze/thaw/vortex four times. Purify the virus by CsCl gradient centrifuge.

IV. Generation of ADV-GFP-CMV-E-ICE (FIGS. 33 and 34)

1. Linearize 1 ug pAdTrack-CMV-E-Fᵥ1-Fᵥᵢₛ1-ICEst with Pme I. Purify it by phenol-chloroform extraction, ethanol precipitation and resuspend in 6 μl H2O.

2. Mix it with 100 μg p AdEasy-1 (FIG. 35) (in 1 μl), Co-transform 20 μl E. coli BJ5183 competent cells with GenePulser at 2,500 V, 200 Ohms, 25 uFD.

3. Pick up 20 smallest colonies. Do miniprep and check with Pac I. Candidate clones usually yield a large fragment (near 30 kb), plus a smaller fragment of 3.0 kb or 4.5 kb.

4. Re-transform the correct recombinant plasmids into Xl1-blue. Midiprep with Qiagen kit.

5. Transfect 293 cell by the recombinant plasmid with FuGene. 4 μg DNA/6 μl FuGene/well (6 well plate). Check GFP expression with fluorescent microscope.

6. Harvest the cells when 30% of them are detached. Spin down the cells, use the supernatant for next infection. Repeat infection for several rounds.

7. Collect the cells, repeat freeze/thaw/vortex four times. Purify the virus by CsCl gradient centrifuge.

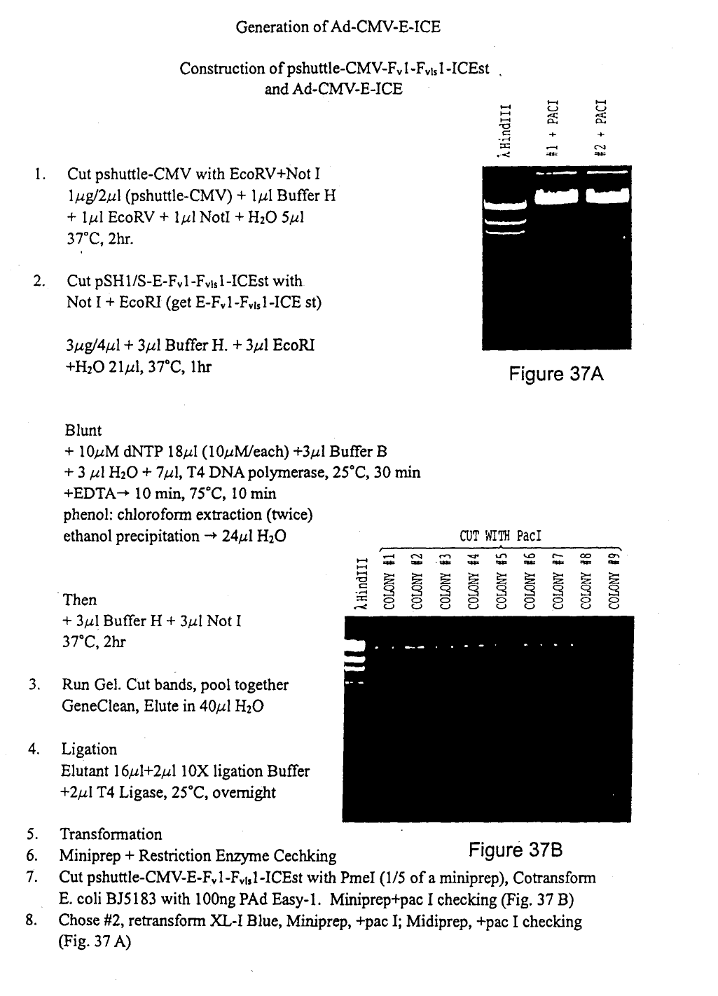

V. Construction of pShuttle-CMV-E-Fᵥ1-Fᵥᵢₛ1-ICEst (FIGS. 37-39)

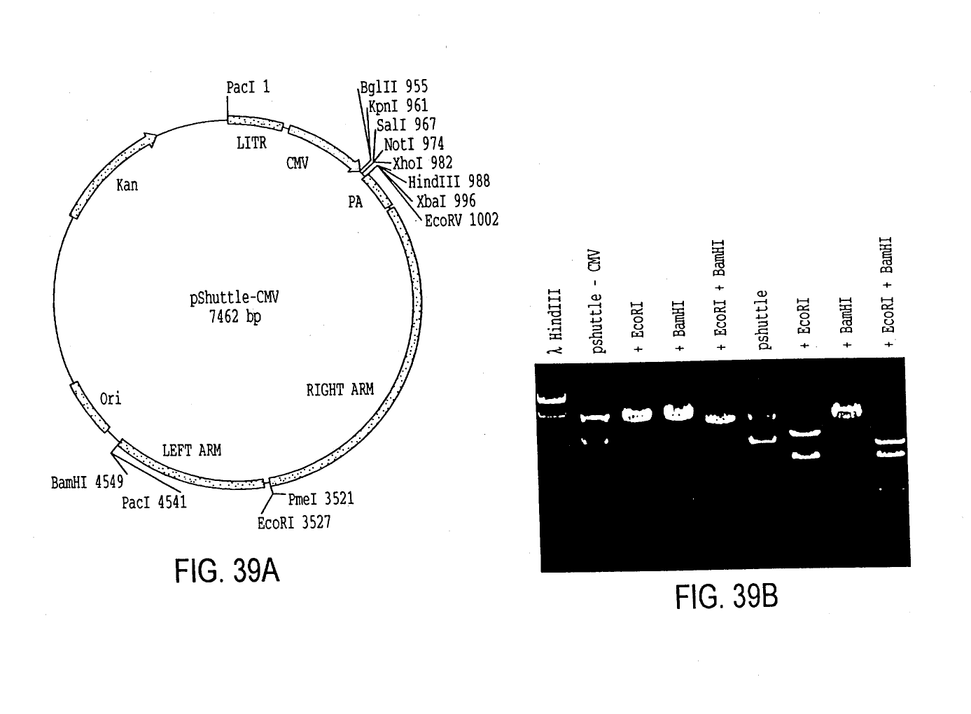

1. Digest pShuttle-CMV (FIG. 39) with Eco RV and Not I. Purify the 7.4 kb vector by agarose electrophoresis and GeneClean.

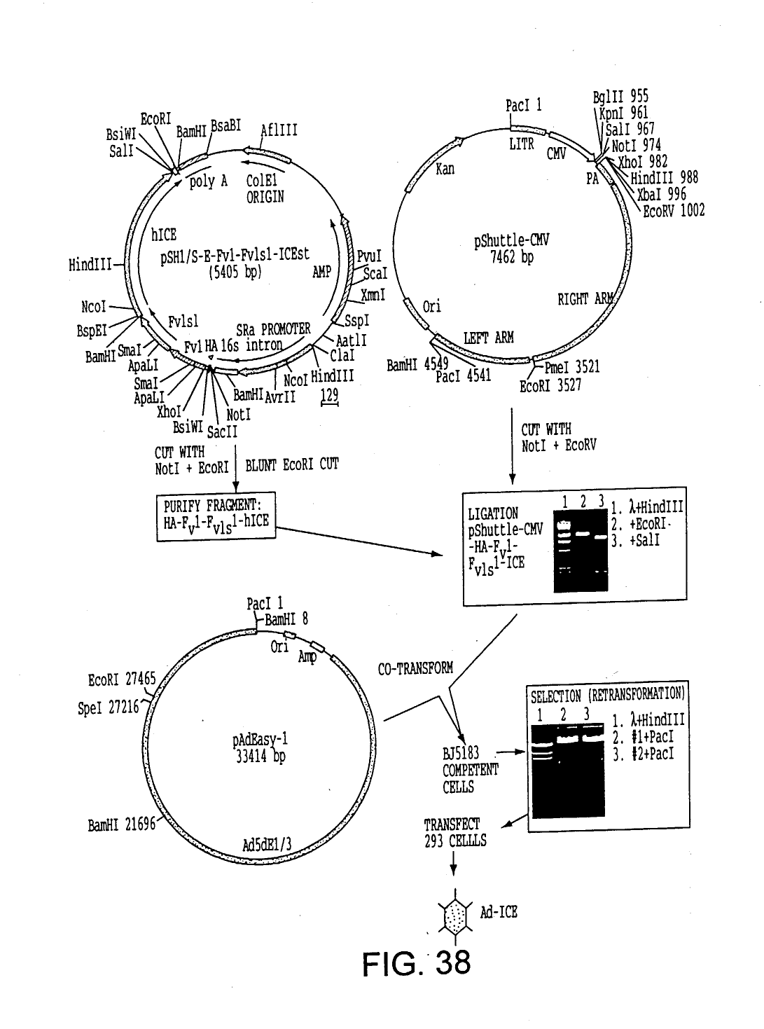

2. Digest pSH1/S-E-Fᵥ1-Fᵥᵢₛ1-ICEst with Eco RI and Not I. Blunt the Eco RI end. Purify the 2.2 kb fragment by agarose electrophoresis and GeneClean. FIG. 38.

3. Ligate the above two fragments and transform XL1-blue with the ligation. Chose several colonies, do miniprep and check with Sal I and Eco RI.

VI. Generation of ADV-CMV-E-ICE (FIGS. 37-39)

1. Linearize 1 μg pShuttle-CMV-E-Fᵥ1-Fᵥᵢₛ1-ICEst with Pme I. Purify it by phenol-chloroform extraction, ethanol precipitation and resuspend in 6 μl H2O.

2. Mix it with 100 μg p AdEasy-1 (in 1 μl), Co-transform 20 μl E. coli BJ5183 competent cells with GenePulser at 2,500 V, 200 Ohms, 25 uFD.

3. Pick up 20 smallest colonies. Do miniprep and check with Pac I. Candidate clones usually yield a large fragment (near 30 kb), plus a smaller fragment of 3.0 kb or 4.5 kb.

4. Re-transform the correct recombinant plasmids into Xl1-blue. Midiprep with Qiagen kit.

5. Transfect 293 cell by the recombinant plasmid with FuGene. 4 μg DNA/6 μl FuGene/well (6 well-plate). Check GFP expression with fluorescent microscope.

6. Harvest the cells when 30% of them are detached. Spin down the cells, use the supernatant for next infection. Repeat infection for several rounds.

7. Collect the cells, repeat freeze/thaw/vortex four times. Purify the virus by CsCl gradient centrifuge.

### EXAMPLE 19

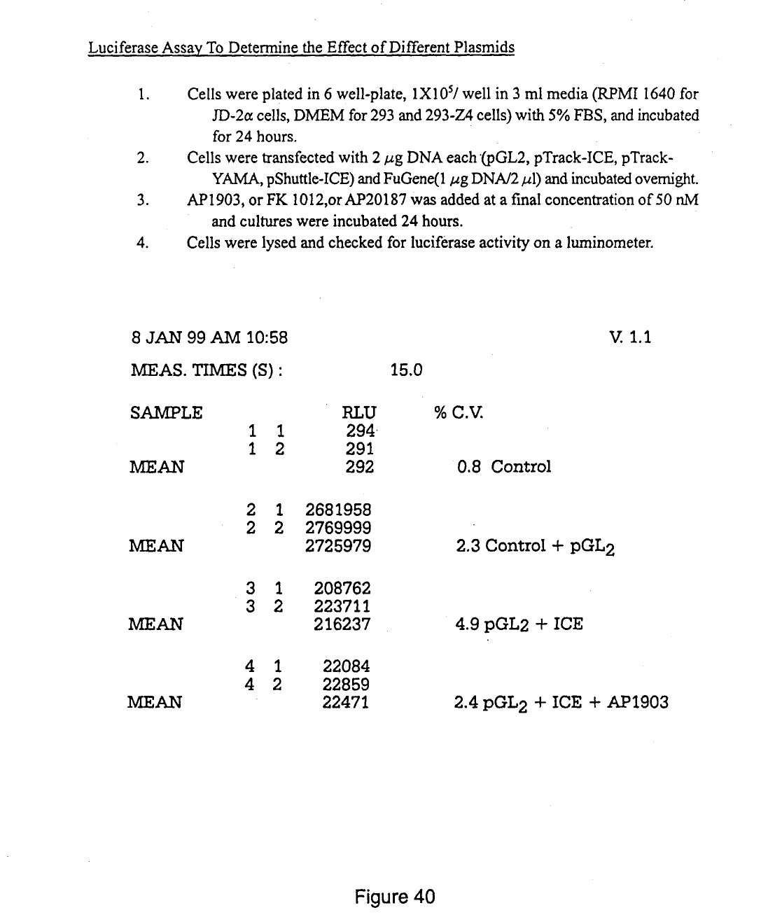

Luciferase Assay To Determine the Effect of Different Plasmids (FIG. 40)

1. Cells were plated in 6 well-plate, 1×10⁵/well in 3 ml media (RPMI 1640 for JD-2a cells, DMEM for 293 and 293-Z4 cells) with 5% FBS, and incubated for 24 hours.

2. Cells were transfected with 2 μg DNA each (pGL2, pTrack-ICE, pTrack-YAMA, pShuttle-ICE) and FuGene (1 μg DNA/2 μl) and incubated overnight.

3. AP1903, or FK 1012, or AP20187 was added at a final concentration of 50 nM and cultures were incubated 24 hours.

4. Cells were lysed and checked for luciferase activity on a luminometer.

The results are shown at FIG. 40.

### EXAMPLE 20

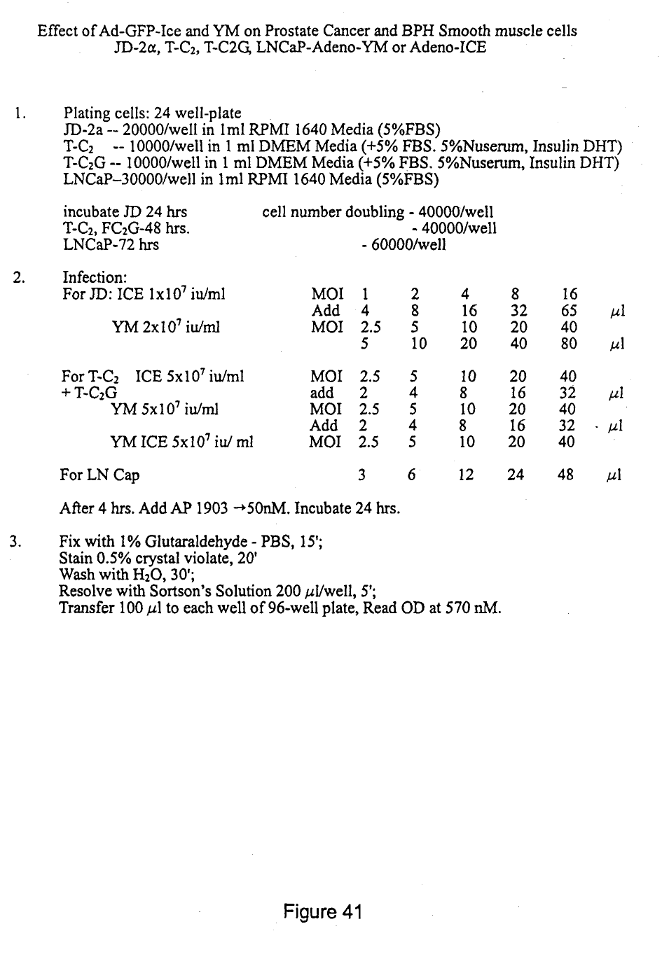

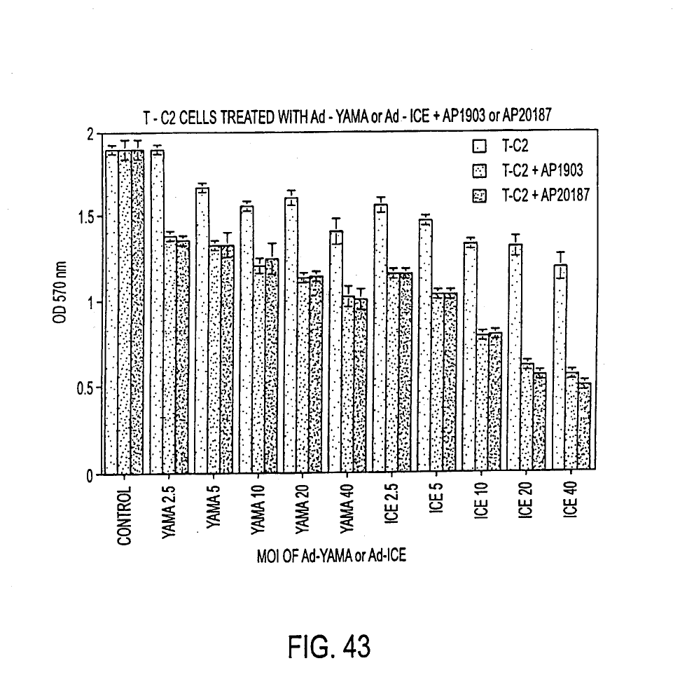

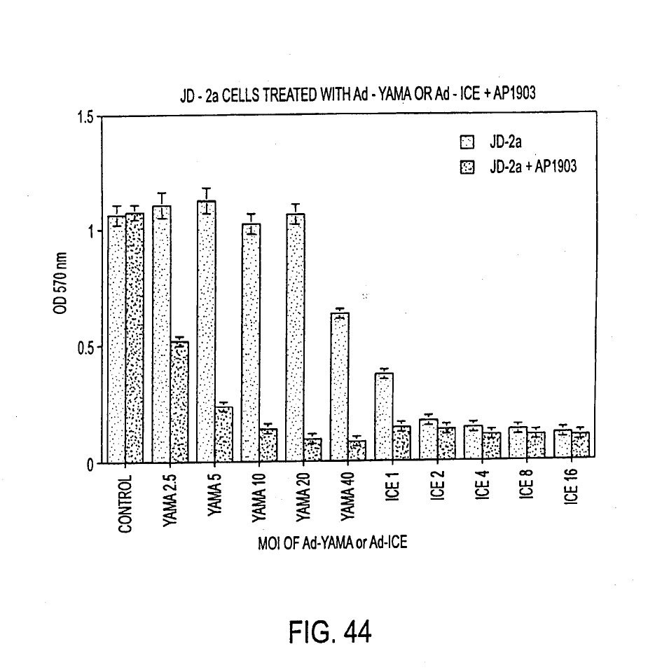

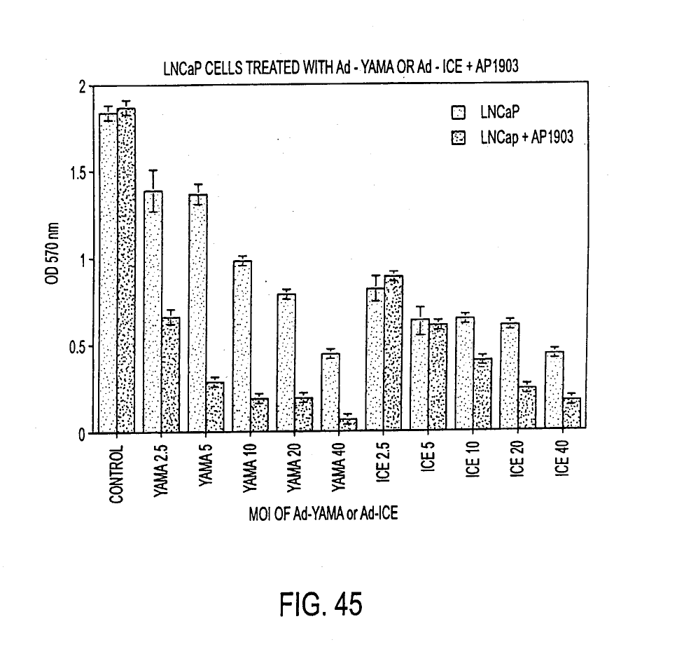

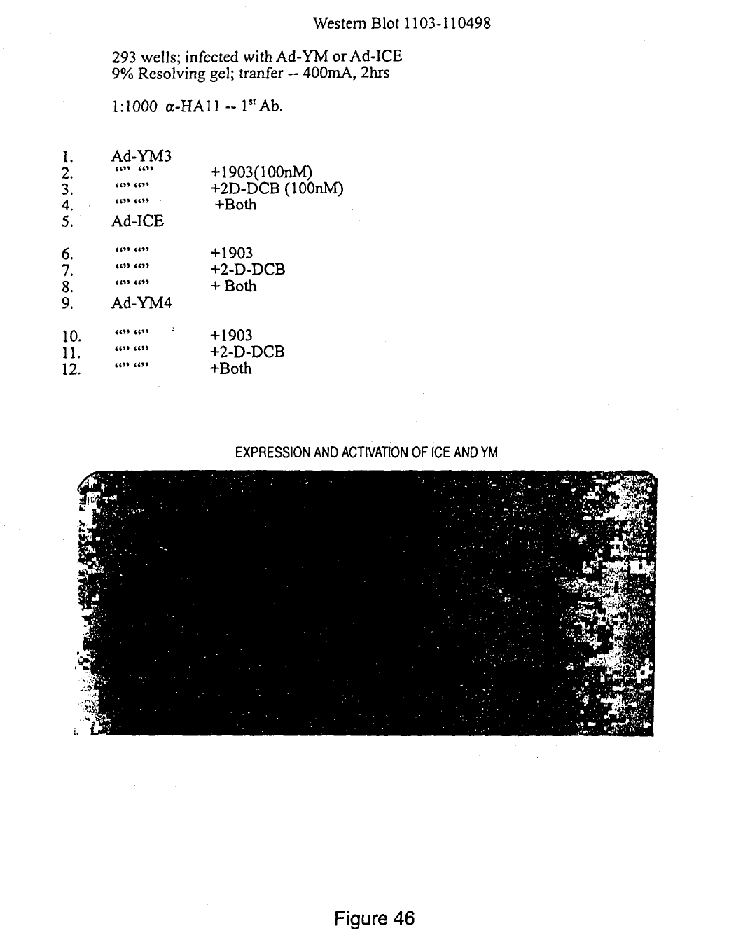

Assay To Determine Effects of Using Different Viruses (FIGS. 41-45)

1. Cells were plated in 24 well-plate, 2×10⁴ to 4×10⁴ cells/well in 1 ml media (RPMI 1640 for JD-2a, LNCaP and PC3 cells; DMEM for Tramp, T-C2 and T-C2G cells) with 5% FBS, and incubated until the cell number doubled.

2. Cells were infected with the virus at different MOI and incubated overnight.

3. AP1903, or FK 1012, or AP20187 was added at a final concentration of 50 nM and cultures were incubated 24 hours.

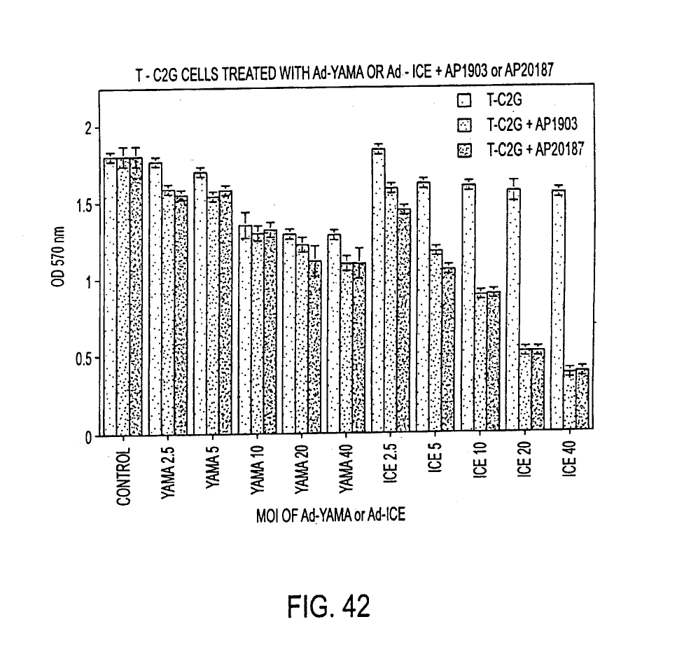

4. Cells were fixed with 1% glutaraldehyde for 15 min.; stained with 0.5% crystal violet for 20 min.; washed with H2O for 30 min.; air dried; resolved with 200-500 ul/well Soreson's Solution for 5 min.; transfered 60-100 μl to each well of 96-well plate and read OD at 570 nm. Results are shown in FIGS. 42-45.

### EXAMPLE 21

Replication deficient (ΔE1) adenoviral vectors expressing green flourescent protein and conditional Caspase 1 (ICE) or Caspase 3 (YAMA) were engineered. These vectors independently express green fluorescent protein so that infected cells are easily identified by their green color under fluorescent microscopy. These vectors were tested for their ability to induce apoptosis in vitro in a SMC line derived from a patient with BPH upon administration of a non-toxic, lipid-permeable, divalent FK506 analog (AP1903).

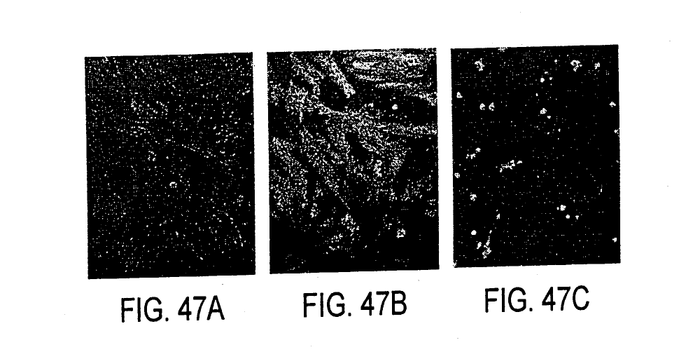

40,000 JD-2a cells per well were plated in 24-well plates and infected at a multiplicity-of-infection (MOI) of ˜25 with Adv-Fᵥ1-YAMA, an adenoviral vector expressing CID-regulated YAMA. After 24 hours, culture media was changed to control media±50 nM AP1903 for an additional 24 hours, and the cells were viewed under fluorescent microscopy. All of the cells incubated with virus and maintained in control media appeared green, and were clearly attached and viable, similar to non-fluorescent non-infected JD-2a cells (FIG. 47b). However, >99% of the cells incubated with virus and maintained in 50 nM AP1903 were either dead or in the process of undergoing apoptosis (FIG. 47c). FIG. 47a shows uninfected, untreated culture of JD-2a cells.

### EXAMPLE 22

ADV-FKBP/ICE effectively kills JD-2a BPH cells and PC-3 Prostate cancer cells

1. Cells were plated in 24 well-plate, 2×10⁴ to 4×10⁴ cells/well in 1 ml RPMI 1640 media with 5% FBS, and incubated until the cell number doubled.

2. Cells were infected with the virus at different MOI and incubated overnight.

3. AP1903 was added at a final concentration of 50 nM and cultures were incubated 24 hours. Control cultures did not receive AP1903.

4. Cells were fixed with 1% glutaraldehyde for 15 min.; stained with 0.5% crystal violet for 20 min.; washed with H2O for 30 min.; air dried; resolved with 200-500 ul/well Soreson's Solution for 5 min.

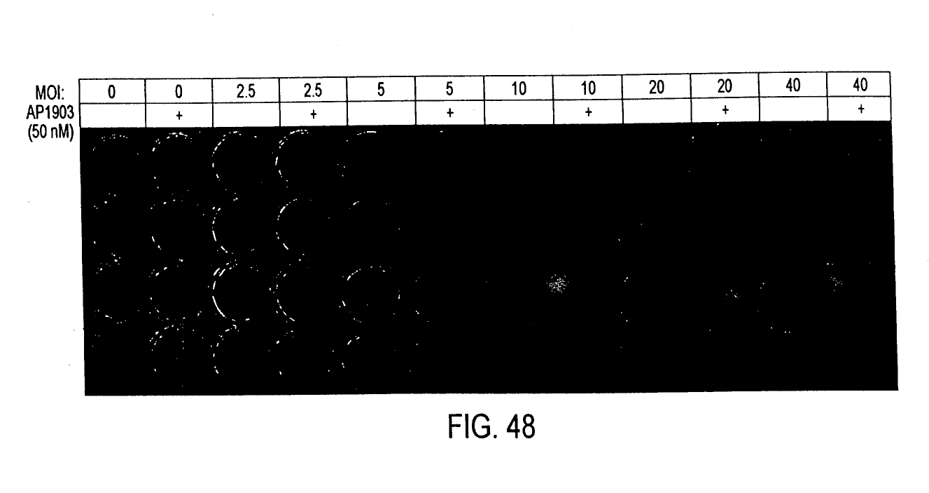

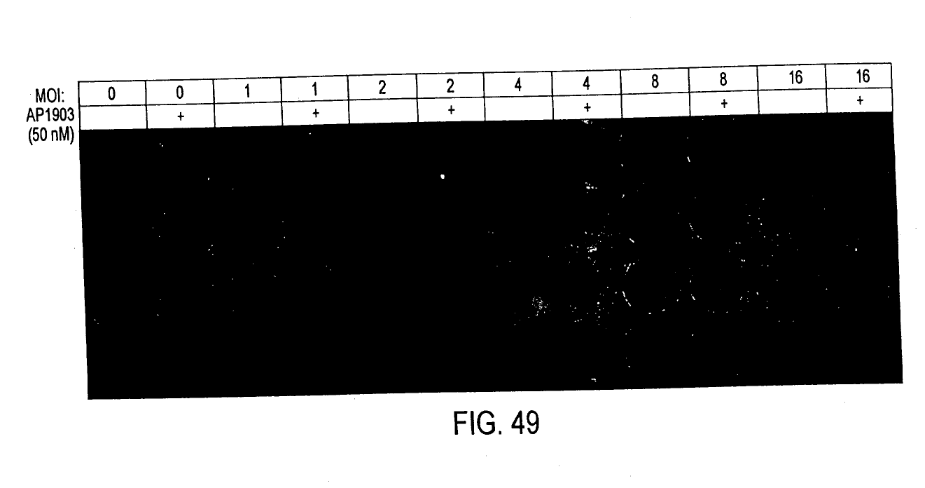

Results are shown in FIGS. 48 and 49. Addition of 50 nM AP1903 is indicated by (+). Figure shows that ADV-FKBP/ICE kills JD-2a BPH cells at higher MOI even without the CID. FIG. 48 shows that ADV-FKBP/ICE effectively kills PC-3 prostate cancer cells at MOIs of 5 and greater upon the administration of the CID.

### EXAMPLE 23

Therapeutic applications of chemically inducible apoptosis factors

In general any therapeutic application currently practiced using the HSV-tk/ganciclovir system can be practiced using the present invention. The chemically inducible apoptosis factors may be incorporated into any delivery vector presently incorporating the HSV-tk gene and the vector may be applied in the same fashion as presently employed. In order to induce apoptosis, the appropriate chemical inducer of dimerization is administered. Specific examples of types of tumor cells that may be treated with gene therapy vectors expressing the ADSs of the present invention are presented below. Therapeutic applications will be developed using the models systems described, or any equivalent model system. Those skilled in the art will readily appreciate that it may be necessary to optimize certain parameters such as the dose of the gene therapy vector, dose of the dimerization ligand and the timing of the application of the dimerization ligand after the inoculation of gene therapy vector. Such optimization is well within the purview of ordinary skill in the art. The examples presented below are for illustrative purposes only are not intended to be an exhaustive recitation of all possible therapeutic applications. Other therapeutic applications will be obvious to those skilled in the art upon reading the present application and are within the scope of this invention.

One example of the use of the chemically inducible apoptosis factors of the present invention is in the treatment of prostate cancer. A gene therapy vector expressing the chemically inducible apoptosis factor can be directly injected into the prostate and then activated with the appropriate ligand. Optionally, the expression of the chemically inducible apoptosis factor may be controlled by the prostate specific antigen (PSA) promoter. In a preferred embodiment, the gene therapy will be a recombinant adenovirus vector. As a model system, mice may be injected with a suitable prostate cancer cell line, such as RM-1. About 4×10⁶ cells may be subcutaneously injected into BALB/c mice to induce tumor formation. After a period of time to allow growth of the tumor, the mice will be injected with a gene therapy vector. In a preferred embodiment the vector will be a recombinant adenovirus vector constructed using the plasmids of FIGS. 19 and 20. After a suitable period of time, apoptosis will be induced in the treated cells by the addition of a chemical inducer of dimerization.

Another example of the use of therapeutic applications of the ADSs of the present invention is the use of gene therapy vectors expressing and ADS to treat gliomas. A rat glioma model system can be constructed by injection of a suitable glioma cell line, such as 9L, into rats to induce tumor formation. The tumor cells may be injected directly into the brain in a stereotactic inoculation of about 1×10⁴ 9L cells. Alternatively, subcutaneous injections of about 1×10⁶ 9L cells may be used. After a suitable time period to allow growth of the tumor, the rats will be inoculated with a gene therapy vector. In a preferred embodiment, the gene therapy vector will be a recombinant adenovirus expressing an ADS. Optionally, the expression of the ADS will be controlled by a tissue specific promoter. After inoculation with gene therapy vector, the animals will be injected with an appropriate dimerization ligand.

A gene therapy vector expressing an ADS of the present invention may be used to treat squamous cell carcinomas. A nude mice model system may be constructed by injection of a suitable squamous carcinoma cell, such as USMSCC29 cells, into nude mice to induce tumor formation. The tumor cells, about 5×10⁶ cells, may be injected into the flanks of the animals. After a suitable time period to allow growth of the tumor, approximately 2 weeks, the mice will be inoculated with a gene therapy vector. In a preferred embodiment, the gene therapy vector will be a recombinant adenovirus expressing an ADS. Optionally, the expression of the ADS will be controlled by a tissue specific promoter. After inoculation with gene therapy vector, the animals will be injected with an appropriate dimerization ligand to induce apoptosis of the tumor. Those skilled in the art will readily appreciate that it may be necessary to optimize certain parameters such as the dose of the gene therapy vector, dose of the dimerization ligand and the timing of the application of the dimerization ligand after the inoculation of gene therapy vector. Such optimization is well within the purview of ordinary skill in the art.

Gene therapy vectors expressing the ADSs of the present invention may be used to treat breast cancer. A suitable model system may be constructed by injecting athymic mice with a suitable breast cancer cell line, such as MDA-MB435A. Tumors may be induced by the intraperitoneal injection of about 5×10⁶ MDA-MB435A cells. After a suitable period to allow tumor formation, about 10 days, a gene therapy vector of the present invention will be injected into the mice. Subsequently, the mice will be injected with the chemical inducer of dimerization to induce apoptosis in the cells carrying the gene therapy vector. In a preferred embodiment, the gene therapy vector will be a recombinant adenovirus expressing the ADSs of the present invention. Optionally, the expression of the ADS may be controlled by a tissue specific promoter.

### EXAMPLE 24

Treatment of benign hyperproliferative disorders using gene therapy vectors expressing ADSs of the present invention.

The gene therapy methods of the prior art that use suicide genes to eradicate cancerous cells are entirely unsuited to applications involving benign hyperproliferative disorders. The high risk associated with the toxic pro-drugs of the prior art restricts the therapeutic applications of these methods to use in life threatening situations, In contrast, gene therapy vectors expressing the ADSs of the present invention are well suited to applications involving the treatment of benign hyperproliferative disorders by virtue of the non-toxic nature of the constructs themselves as well as the non-toxic nature of the chemical inducer of dimerization.

BPH is one example of a benign hyperproliferative disorder that is amenable to treatment using gene therapy methods based on the ADSs of the present invention. As demonstrated in Examples 6-8, gene therapy vectors expressing the ADSs of the present invention are extremely effective in killing a wide variety of cell types, including those derived from BPH. A gene therapy vector expressing an ADS may directly injected into a prostate gland of a patient suffering from BPH. After a suitable period of time to allow the gene therapy vector to be taken up by the treated cell, the patient will be given a chemical inducer of dimerization to induce apoptosis in the treated tissue. In a preferred embodiment, the gene therapy vector will be a recombinant adenovirus expressing an ADS. In other preferred is embodiments the expression of the ADS will be under the control of a prostate specific promoter. In a most preferred embodiment, the expression of the ADS will be controlled by the prostate specific antigen promoter.

### EXAMPLE 25

Construction of tumor specific ADSs.

The ADSs of the present invention can be used to specifically ablate cells of tumors by incorporating tumor specific promoters into the gene therapy vectors. The expression of the ADS will be placed under the control of a promoter that is active only in the target tumor cells.

For example, to construct a gene therapy vector that specifically ablates melanoma cells, the expression of the ADS of the present invention can be placed under the control of the tyrosinase promoter (Vile, et al. Cancer Res. 53:962-967, 1993). The construction of a melanoma specific, gene therapy vector can be accomplished using techniques well known in the art. The tyrosinase promoter can be operatively connected to a cassette comprising one or more chemical inducer binding domains fused in frame to a protein that induces apoptosis upon dimerization. The cassette, including the tyrosinase promoter, is then inserted into a plasmid for recombination into an adenovirus. Typically this is accomplished by inserting the cassette into a plasmid that contains a copy of the adenovirus E1 gene.

The cassette is inserted into the E1 gene such that portions of the E1 gene flank both ends of the cassette. The resulting plasmid is used to transfect cells infected with adenovirus resulting in the inclusion of the cassette into the adenovirus by homologous recombination. The construction of adenoviruses incorporating heterologous genes by this method is well known to those of skill in the art. The method used by Chen, et al. (PNAS 92: 2577-2581, 1995, which is specifically incorporated herein by reference) for the construction of an adenovirus expressing the HSV-tk gene may be used by substituting the gene encoding the ADS of the present invention for the HSV-tk gene.

Those skilled in the art will readily appreciate that gene therapy vectors that specifically target other types of tumors can be constructed by the use of promoters specific for the type of tumor targeted. Others tumor specific promoters that may be used in the present invention include, but are not limited to, the prostate specific antigen promoter for targeting prostate tumors (Ko, et al. Proc. Am. Assn. Cancer Res. 37:349, 1996), the human surfactant protein A promoter for targeting non-small-cell lung carcinomas (Smith, et al. Hum. Gene Therapy 5:29-35, 1994), the glucose related protein 78 (grp78) promoter for targeting fibrosarcomas (Gazit, et al. Cancer Res. 55:1660-1665, 1995) and the carcinoembryonic antigen (CEA) promoter to target tumor cells like pancreatic carcinoma cells (DiMaio, et al. Surgery 116:205-213, 1994). Any promoter that is substantially more active in tumor cells than in non-tumor cells may be used to practice the present invention.

### EXAMPLE 26

Caspase-based suicide genes can trigger apoptosis in prostate cancer cell lines derived from tumors from intact and castrated TRAMP and MPR model mice.

Design:

We will determine whether progression to androgen-independence affects sensitivity to caspase-mediated apoptosis. Apoptosis assays will be performed on prostate cancer cells derived from both the TRAMP model and the MPR model following CID-mediated caspase activation. The efficacy of caspase-mediated apoptosis in clones from intact mice will be compared to TRAMP tumors isolated from castrated mice or RM cells passaged in castrated mice. Finally, direct bystander killing will be investigated.

Methods:

TRAMP-C2 cells (Foster, B. A., et al. (1997) Cancer Res 57, 3325-3330), RM-1 and RM-9 cells (Baley, P. A., et al. (1995) J Steroid Biochem Mol Biol 52, 403-413) will be transduced with recently constructed retrovirus or adenovirus (ADV) vectors expressing CID-responsive ICE and YAMA proteins. Twenty-four hours after transduction, cells will be treated with up to 100 nM AP1903. Control cells will be mock transduced or mock treated. Alternatively, these experiments will be performed in tumors from castrate mice. TRAMP mice will be castrated at twelve weeks and androgen-independent tumors will be removed at 24 weeks as described previously (Gingrich, J. R., et al. (1997) Cancer Res 57, 4687-4691). Androgen-independent RM cell lines have been previously characterized (Baley, P. A., et al. (1995) J Steroid Biochem Mol Biol 52, 403-413). Twenty hours after CID treatment, cells will be analyzed for apoptosis by DNA laddering, annexin V-FITC staining followed by flow cytometry, and by the TUNEL assay. Bystander killing will be based on the minimum fraction of FKBP/caspase-expressing cells needed to trigger apoptosis in >95% of the cells in a confluent culture following CID treatment. We will test apoptosis by multiple assays because the exact biochemical changes during apoptosis can differ in distinct tissues.

Expected Results:

Since we have previously demonstrated that TRAMP-C2 cells, RM-1 and RM-9 cells are sensitive to CIDs following transient transfection of CID-responsive ICE and YAM A plasmids, virally transduced cells should be similarly sensitive to CIDs. Further, progression to androgen-independence should not effect ICE and YAMA sensitivity because these caspases can trigger apoptosis even in the presence of relatively high levels of Bcl-X_{L}. The observation of a bystander effect in culture will be novel, as it has not been previously reported.

### EXAMPLE 27

ADV-FKBP/ICE effectively kills TRAMP-C2 cells in vivo

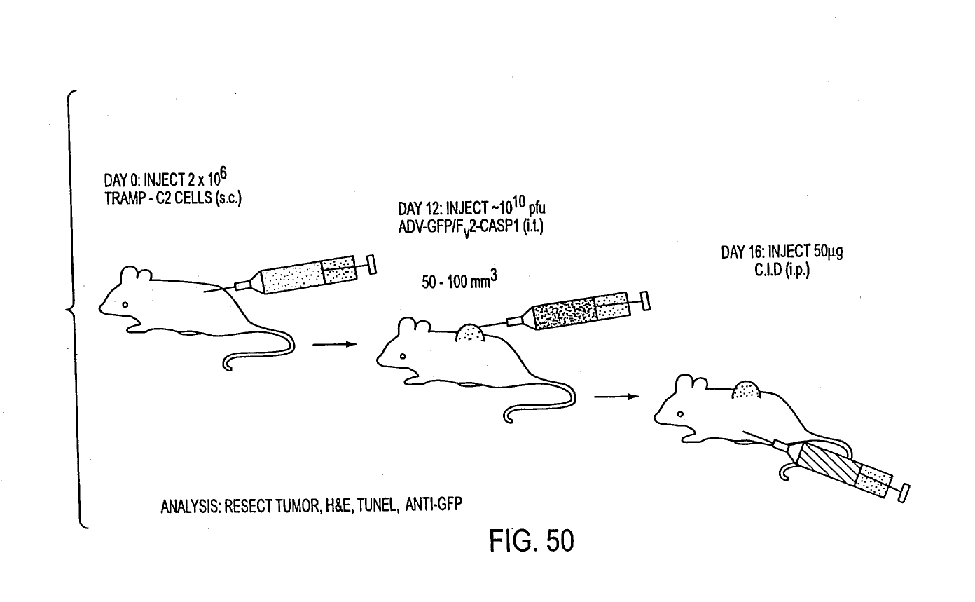

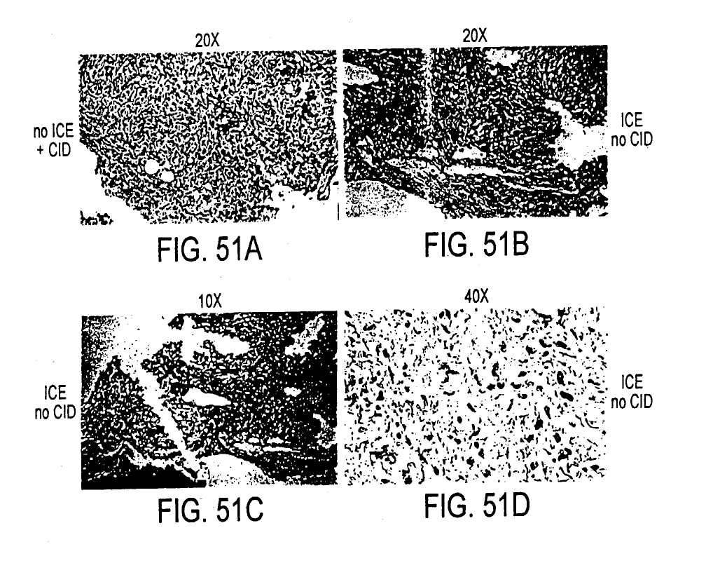

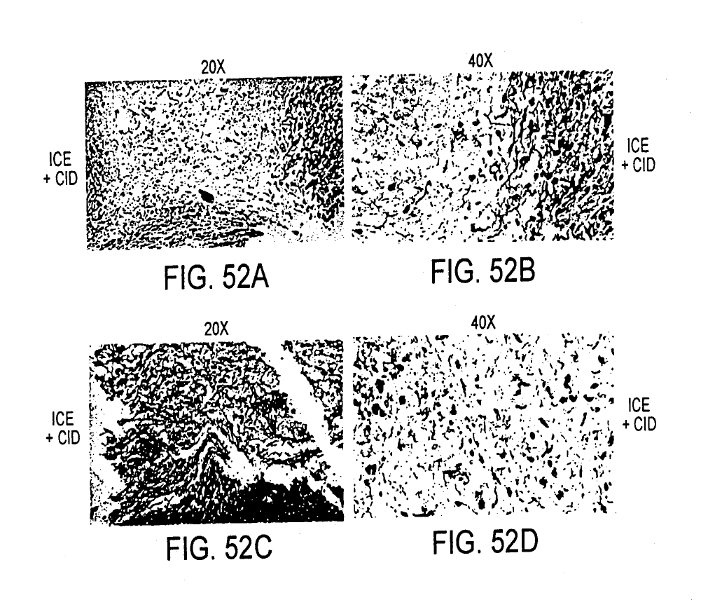

Referring to FIG. 50, mice were subcutaneously injected with 2×10⁶ TRAMP-C@ cells to induce tumor formation. On day 12, tumors were injected with ˜10¹⁰ of ADV-GFP/Fᵥ2-Casp1. On day 16, the mice were intraperitoneally injected with 50 μg of CID. Control cells were be mock transduced or mock treated. Twenty hours after CID treatment, tumors were resected, and analyzed. FIGS. 51 and 52 show transduced, untransduced, treated and untreated tumor sections. Referring to FIG. 51, the tumor section showing no ICE+CID appears healthy; while the tumor sections treated with ICE and no CID are showing the effects of apoptosis. FIG. 52 shows the dramatic apoptotic effect that results upon administration of a CID.

### EXAMPLE 28

Determine the safety and efficacy of gene therapy using ADV vectors expressing inducible ICE and YAMA in the TRAM and MPR orthotopic prostate cancer models.

Design:

We plan to perform in vivo gene therapy studies similar to those previously reported using HSV-tk/GCV in the MPR model system (Eastham, J. A., et al. (1996) Hum Gene Ther. 7, 515-523). Subcutaneous tumors will be generated by injection of RM-1 cells into syngeneic, C57BL/6 male hosts and inoculated with escalating doses of HSV-tk virus, inducible YAMA virus, inducible ICE virus, or a control β-gal virus (5×10⁷ to 1×10⁹ pfu). The mice will receive GCV (HSV/tk arm) or AP1903 (ICE/YAMA arms) twice daily for 6 days and will be sacrificed when tumor volumes exceed 2.5 cm³ or when they appear in distress. Tumors will be assessed for final volume, and histologically for apoptotic index and extent of tumor necrosis. Finally, mean survival in days will be compared in the four treatment arms.

Methods:

4×10⁶ RM-1 cells will be injected s.c. in 12-week-old C57BL/6 mice. Twenty mice will be injected in each treatment arm. Tumor volumes will be calculated by the formula for a rotational ellipsoid. To ascertain a target therapeutic viral dose, escalating viral doses from 5×10⁷ to 1×10⁹ pfu will be injected directly into tumors when the volume is approximately 50 mm³. Twelve hours following viral injection, each animal will be treated with intraperitoneal (ip) infjectfions of GCV at a dose of 10 mg/kg body weight or AP1903 at a dose of 2 mg/kg body weight every 12 hr for 6 days. Tumor volume will be assessed every other day, and mice will be sacrificed when tumor volume exceeds 2.5 cm³. Tumors will be assessed histologically or with the TUNEL technique to label cells undergoing apoptosis.

Expected Results:

It is anticipated that inducible ICE/YAMA constructs will be able to trigger apoptosis more extensively and more quickly in prostate cancer cells, due to their slow growth-relative to other tumor types and the ability of caspases to trigger rapid apoptosis.

### EXAMPLE 29

Triggering apoptosis in orthotopic tumor cells will reduce the number of spontaneous metastasis.

Design:

We plan to perform in vivo gene therapy studies similar to those previously reported using HSV-tk/GCV in the orthotopic MPR model system (Hall, S. J., et al. (1997) Int. J Cancer 70, 183-187). Orthotopic tumors will be generated by injection of RM-1 cells into the prostates of syngenic male hosts. Typically this aggressive model of prostate cancer results in distress or death of the host by 16-17 days post-inoculation. In contrast to the s.c. model (SA2), orthotopic tumors result in documented metastatic activity in over 80% of animals by 16-17 days with the highest activity in the pelvic and retroperitoneal (RP) lymph nodes and the lowest activity in the lung. Since tumors are metastatic by 2 weeks post-inoculation, at 7 days post-inoculation, tumors will be injected with an appropriate dose, determined in SA2, of HSV-tk virus, inducible YAMA virus, inducible ICE virus, or a control β-gal virus. The mice will receive GCV (HSV/tk arm) or AP1903 (ICE/YAMA arms) twice daily for 6 days, and sacrificed at 14 days. A careful autopsy for gross and microscopic metastasis will be performed. Survival studies will be performed with animals sacrificed when in distress. Mean survival in days will be compared in the four treatment arms.

Methods:

Initially, 1000 RM-1 cells in 10 μl will be injected directly into the right or left lobe of the dorsolateral prostate of adult syngeneic male C57BL/6 mice. 20 mice in each treatment arm will be injected. Tumor volumes will be calculated as above. Tumors will be weighed, and the pelvic and RP lymph nodes and samples of lungs will be excised and processed histologically. Animals will be scored as having metastasi if any lymph node and/or lung has microscopic evidence of metastasis.

Expected Results:

We expect to see a reduction in spontaneous metastasis, which is inversely proportional to the efficiency of the method of killing. Since the efficacy of HSV-tk/GCV treatment can be reduced by checkpoint proteins like Bcl-2, which are also associated with progression to metastatic cancer, we expect Bcl-2-insensitive, caspase-mediated apoptosis to be a more anti-metastatic.

### EXAMPLE 30

Triggering apoptosis in s.c. tumor cells will augment a systemic immune response against a second-site tumor.

Design:

Tail vein inoculum challenges will be performed to ascertain whether system anti-metastatic activity can be induced against a second tumor challenge following a single treatment with inducible-YAMA/AP1903 or inducible-ICE/AP1903, as compared to HSV-tk/GCV as previously reported (Hall, S. J., et al. (1997) Int. J Cancer 70, 183-187).

Methods:

Using the s.c. model, tumors will be initiated as described in SA2, and treated appropriately with either GCV or AP1903 for 6 days. On day 10 post-tumor inoculation, s.c. tumors will be surgically removed. Two weeks later, 40,000 RM-1 cells will be injected via the dorsolateral tail vein. Animals will be euthanized 2 weeks later. The lungs will be removed and fixed in Bouin's solution. Individual visible lung metastases will be counted with the aid of a dissecting microscope, and the three treatment arms compared.

Expected Results:

We anticipate a larger reduction in metastasis after caspase treatment relative ot HSV-tk treatment. This should reflect a more robust anti-tumor immune response initiated by the efficient transfer of putative tumor antigens to APCs following caspase-mediated apoptosis. Since s.c. tumors do not have the same local environment as orthotopic tumors with regards to extracellular matrix factors, microvasculature and local APCs, we will repeat these experiment in an orthotopic model if significant anti-metastatic effects (or differences) are not seen.

### EXAMPLE 31

Construction of animals specifically deleted in various cell types.

The present invention can be used to create animals that are specifically deleted of a certain cell type. A recombinant animal, for example a mouse, can be constructed so that an ADS under the control of a tissue specific promoter is stably incorporated into the genome. This will result in an animal that expresses an ADS in a single cell type. In the absence of a chemical inducer of dimerization, the cells expressing the ADS will develop normally. When desired, the specific cells may be deleted by the addition of the inducer.

Animals of this type will permit the elucidation of the roles of various types of cells. This will be particularly useful in the elucidation of the roles of cells of the immune system. By varying the timing of the deletion of the cell type expressing the ADS, the role of that cell type in development may also be ascertained.

Examples of types of cells that might be specifically deleted include, but are not limited to, β-islet cells of the pancreas to develop a diabetes model and melatonin-containing cells of the substantia nigra to develop a model for Parkinson's disease. This approach will also be useful in studying the roles of various cells of the immune system. Other cell types that may be specifically deleted include, but are not limited to, cardiac myocytes to create a model for cardiac disease, thyroid cells for a hypothyroidism model, pituitary cells for growth hormone deficiencies, osteoblasts for osteoporosis, kidney cells for renal failure, liver cells for hepatitis and the cells of any endocrine organ.

Transgenic animals will be made using techniques well known to those of skill in the art. In brief, an mammalian expression vector will be micro-injected into the male pro-nuclei of a fertilized embryo. The mammalian expression cassettes will typically include the cDNA encoding the ADS subcloned 3′ of a tissue specific promoter/enhancer sequence. In a preferred embodiment, the tissue specific promoter/enhancer sequence will be followed by a splicing donor acceptor sequence. Several reports demonstrate that splicing is important for efficient mRNA processing and nuclear export. The mammalian expression vector may also include a polyadenylation sequence 3′ to the DNA sequence encoding the ADS.

Injected embryos will be implanted into pseudo-pregnant females. Tail DNA from all live pups will be analyzed for integration. Transgenic “founder” mice will be further bred and analyzed for germline transmission of the DNA.

### EXAMPLE 32

Treatment of arteriosclerosis using adenoviruses expressing an ADS.

Recombinant adenoviruses expressing ADS can be used to treat atherosclerosis. Atherosclerosis is characterized by a proliferation of smooth muscle cells. As demonstrated by their ability to kill smooth muscle cells derived from BPH, the chemically inducible apoptosis factors of the present invention may be used to ablate the smooth muscle cells present in arteriosclerotic tissue. A gene therapy vector expressing an ADS of the present invention may be directly applied to the interior wall of a sclerotic vessel using methods known to those skilled in the art. An example of such a method is provided by Nabel, et al. U.S. Pat. Nos. 5,698,531, 5,328,470 and 5,707,969 which are specifically incorporated herein by reference. In brief, a solution containing the gene therapy vectors of the present invention is delivered to the sclerotic portion of the vessel by using a catheter. The specific segment is isolated and the solution is infused into the space adjacent to the lesion for a period of time sufficient to permit the uptake of the vector into the target tissue. Subsequently, dimerization of the ADS is induced by application of the appropriate ligand and the cells taking up the gene therapy vector will undergo apoptosis.

### EXAMPLE 33

Use of ADSs as safety switches in gene therapy vectors.

The previous examples had shown the utility of the ADSs of the present invention as the primary active agent in the treatment of various disorders. In addition to their use in this fashion, the ADSs of the present invention may be incorporated into gene therapy vectors as safety switches. This mode of use will be particularly important in gene replacement therapies.

Gene replacement therapies differ from the preceding examples in that stable, long-term expression of the replacement gene is required. Gene replacement therapies are generally most applicable to those disorders caused when a single gene is either absent or malfunctioning. A gene therapy vector expressing a functional allele of the missing/malfunctioning gene is introduced into the affected cells. To ensure the required long-term expression, replacement therapies typically are conducted using retroviruses as gene therapy vectors with the result that the replacement gene is inserted into the genome of the treated cell. In the process of inserting the replacement gene into an affected cell, there is a possibility that some of the insertions may result in a malignant transformation of the cell.

The present invention is well suited to provide a necessary measure of safety in this case. Genes encoding the ADSs of the present invention may be incorporated into the retroviral gene therapy vector along with the therapeutic gene so that stable integration of the retrovirus results in the expression of both genes. In the event that a malignant transformation occurs, a chemical inducer of dimerization can be administered to delete the cells that contain the retrovirus.

### CONCLUSION

As gene therapy comes of age and vectors move from the laboratory to the clinic, the need for safety is becoming a serious consideration. The instant invention, ADSs based upon apoptosis factors, may lead to clinically suitable suicide switches for these vectors for the following reasons: (i) They can be made exclusively from syngeneic proteins, reducing the likelihood of triggering an immune response; (ii) they are effective in a wide variety of cells, are not restricted to dividing cells, and are not significantly blocked by intracellular checkpoint genes, such as Bcl-x_{L}; and (iii) CIA works with a panel of distinct dimerizing agents that are not currently used for any other purpose and will therefore be useful for regulating viability in multiple independent target tissues (5,8). Finally, CIA may be useful for developmental studies or for treating both malignant and benign hyperproliferative disorders, such as cancer and BPH.

The present invention has been described in terms of preferred embodiments. Those skilled in the art will readily appreciate that these embodiments are for illustrative purposes and are not intended to limit the present invention in any way. Various modifications or changes will be suggested to those skilled in the art by the present application and such modifications are within the spirit of the present invention and within the scope of the appended claims. All publications mentioned in this disclosure are specifically incorporated herein by reference.

### REFERENCES

1. Moolten, F. L. (1986) Cancer Res. 46, 5276-5281.

2. Culver, K. W., Ram, Z., Wallbridge, S., Ishii, H., Oldfield, E. H. & Blaese, R. M. (1992) Science 256, 1550-1552.

3. Matthews, T. & Boehme, R. (1988) Rev. Infect Dis. 10 Suppl 3, S490-S494.

4. Ogasawara, J., Suda, T. & Nagata, S. (1995) J. Exp. Med 181, 485-491.

5. Spencer, D. M., Belshaw, P. J., Chen, L., Ho, S. N., Randazzo, F., Crabtree, G. R. & Schreiber, S. L. (1996) Current Biology 6, 839-847.

6. Liles, W. C., Kiener, P. A., Ledbetter, J. A., Aruffo, A. & Klebanoff, S. J. (1996) J. Exp. Med 184, 429-440.

7. Ogasawara, J., Watanabe-Fukunaga, R., Adachi, M., Matsuzawa, A, Kasugai, T., Kitamura, Y., Itoh, N., Suda, T. & Nagata, S. (1993) Nature 364, 806-809.

8. Belshaw, P. J., Spencer, D. M., Crabtree, G. R. & Schreiber, S. L. (1996) Chem. Biol. 3, 731-738.

9. Smith, C. A., Farrah, T. & Goodwin, R. G. (1994) Cell 76, 959-962.

10. Yuan, J. (1997) Curr. Opin. Cell Biol. 9, 247-251.

11. Hengartner, M. O. (1997) Nature 388, 714-715.

12. Kroemer, G. (1997) Nat. Med. 3, 614-620.

13. Spencer, D. M., Wandless, T. J., Schreiber, S. L. & Crabtree, G. R. (1993) Science 262, 1019-1024.

14. Spencer, D. M. (1996) TIG 12, 181-187.

15. Crabtree, G. R. & Schreiber, S. L. (1996) Trends. Biochem. Sci. 21, 418-422.

16. Duan, H., Chinnaiyan, A. M., Hudson, P. L., Wing, J. P., He, W. W. & Dixit, V. M. (1996) J. Biol Chem 271, 1621-1625.

17. Rosen, M. K., Yang, D., Martin, P. K. & Schreiber, S. L. (1993) JACS 115, 821-822.

18. Spencer, D. M., Graef, I., Austin, D. J., Schreiber, S. L. & Crabtree, G. R. (1995) Proc. Natl. Acad. Sci. USA 92, 9805-9809.

19. Northrop, J. P., Ulhman, K. S. & Crabtree, G. R. (1993) J. Biol Chem 268, 2917-2923.

20. Henkart, P. A. (1996) Immunity 4, 195-201.

21. Boldin, M. P., Varfolomeev, E. E., Pancer, Z., Mett, I. L., Camonis, J. H. & Wallach, D. (1995) J. Biol. Chem. 270, 7795-7798.

22. Irmler, M., Thome, M., Hahne, M., Schneider, P., Hofmann, K., Steiner, V., Bodmer, J. L., Schroter, M., Burns, K., Mattmann, C., Rimoldi, D., French, L. E. & Tschopp, J. (1997) Nature 388, 190-195.

23. Deveraux, Q. L., Takahashi, R., Salvesen, G. S. & Reed, J. C. (1997) Nature 388, 300-304.

24. Freiberg, R. A., Spencer, D. M., Choate, K. A, Duh, H. J., Schreiber, S. L., Crabtree, G. R. & Khavari, P. A. (1997) J. Invest. Dermat. 108, 215-219.

25. Aggarwal, B. B., Singh, S., LaPushin, R. & Totpal, K. (1995) FEBS Letters 364, 5-8.

26. Freiberg, R. A., Spencer, D. M., Choate, K. A., Peng, P. D., Schreiber, S. L., Crabtree, G. R. & Khavari, P. A. (1996) J. Biol. Chem. 271, 31666-31669.

27. Veis, D. J., Sentman, C. L., Bach, E. A. & Korsmeyer, S. J. (1993) J. Immunol 151, 2546-2554.

28. Pan, G., O'Rourke, K., Chinnaiyan, A. M., Gentz, R., Ebner, R., Ni, J. & Dixit, V. M. (1997) Science 276, 111-113.

29. Boldin, M. P., Goncharov, T. M., Goltsev, Y. V. & Wallach, D. (1996) Cell 85, 803-815.

30. Muzio, M., Chinnaiyan, A. M., Kischkel, F. C., O'Rourke, K., Shevchenko, A., Ni, J., Scaffidi, C., Bretz, J. D., Zhang, M., Gentz, R., Mann, M., Krammer, P. H., Peter, M. E. & Dixit, V. M. (1996) Cell 85, 817-827.

31. Tewari, M., Quan, L. T., O'Rourke, K., Desnoyers, S., Zeng, Z., Beidler, D. R., Poirier, G. G., Salvesen, G. S. & Dixit, V. M. (1995) Cell 81, 801-809.

32. Chinnaiyan, A. M., O'Rourke, K., Tewari, M. & Dixit, V. M. (1995) Cell 81, 505-512.

33. Bauer, M. K., Wesselborg, S. & Schulze-Osthoff, K. (1997) FEBS Lett. 402, 256-258.

34. Chinnaiyan, A. M., Chaudhary, D., O'Rourke, K., Koonin, E. V. & Dixit, V. M. (1997) Nature 388, 728-729.

35. Zou, H., Henzel, W. J., Liu, X., Lutschg, N & Wang, X. (1997) Cell 90, 405-413.

36. Li, P., Nijhawan, D., Budihardjo, I., Srinivasula, S. M., Ahmad, M., Alnemri, E. S. & Wang, X. (1997) Cell 91, 479-489.

37. Chinnaiyan, A. M., O'Rourke, K., Lane, B. R. & Dixit, V. M. (1997) Science 275, 1122-1126.

38. Wu, D., Wallen, H. D., Inohara, N. & Nunez, G. (1997) J Biol. Chem. 272, 21449-21454.

39. Boulikas, T. (1997) Anticancer Research 17, 1471-1506.

What is claimed is:

1. A conditionally lethal molecule comprising a chemical inducer binding domain and an apoptosis inducing factor, wherein said apoptosis inducing factor is an apoptosis signal transducing factor.

2. A conditionally lethal molecule according to claim 1, wherein said apoptosis inducing factor is an adaptor molecule.

3. A conditionally lethal molecule according to claim 1, wherein said apoptosis inducing factor is a protease.

4. A conditionally lethal molecule according to claim 1, wherein said apoptosis inducing factor is a caspase.

5. A nucleic acid molecule encoding the conditionally lethal molecule of any one of claims 1-4.

6. A nucleic acid molecule according to claim 5, further comprising a sequence coding for tissue specific expression operatively linked to a sequence coding for a conditionally lethal molecule.

7. A gene therapy vector comprising a nucleic acid sequence coding for the expression of a conditionally lethal molecule according to anyone of claims 1-4.

8. A gene therapy vector according to claim 7, further comprising a sequence coding for a therapeutic gene.

9. A gene therapy vector according to claim 7, further comprising a sequence coding for tissue specific expression operatively linked to a sequence coding for a conditionally lethal molecule.

10. A transgenic animal expressing a conditionally lethal molecule according to any one of claims 1-4.

11. A method of making a transgenic animal comprising the step of micro-injecting a nucleic acid molecule encoding a conditionally lethal molecule according to any one of claims 1-4.

12. A method of treating a disease comprising the step of administering a vector that encodes a conditionally lethal molecule according to any one of claims 1-4.

13. A method according to claim 12, wherein the disease is a hyperproliferative disease.

14. A method according to claim 13, wherein the hyperproliferative disease is a benign disease.

15. A method according to claim 14, wherein the disease is a malignant disease.

16. A method according to claim 12, wherein the disease is atherosclerosis.

17. A method of causing regression of a tumor comprising transfecting cells of said tumor with a nucleic acid molecule encoding a conditionally lethal molecule according to any one of claims 1-4.

18. A method according to claim 17 further comprising administering a CID.

19. A method of causing a reduction in tumor size comprising transfecting cells of said tumor with a nucleic acid molecule encoding a conditionally lethal molecule according to any one of claims 1-4.

20. A method according to claim 19 further comprising administering a CID.

21. A method of causing a reduction in PSA levels comprising transfecting cells of a tumor with a nucleic acid molecule encoding a conditionally lethal molecule according to any one of claims 1-4.

22. A method according to claim 21 further comprising administering a CID.

23. A method of affecting the rate of cell proliferation caused by BPH comprising transfecting prostate cells with a nucleic acid molecule encoding a conditionally lethal molecule according to any one of claims 1-4.

24. A method according to claim 23 further comprising administering a CID.

25. A method of inducing apoptosis in a cell comprising transfecting said cell with a nucleic acid molecule encoding a conditionally lethal molecule according to any one of claims 1-4.

26. A method according to claim 26 further comprising administering a CID.

27. A method for determining the biological role of a cell type, comprising transfecting a cell of said cell type with a nucleic acid molecule encoding a conditionally lethal molecule according to any one of claims 1-4 and administering a CID.
